SEQUENCE LISTING (60 sequences)

SEQ ID NO: 1 - protein, 10 aa, synthetic construct
GFTFEYFGLQ

SEQ ID NO: 2 - protein, 17 aa, synthetic construct
YTSSGGNYEYYEETLKG

SEQ ID NO: 3 - protein, 6 aa, synthetic construct
TKTGDY

SEQ ID NO: 4 - protein, 11 aa, synthetic construct
RASQDIGNSLT

SEQ ID NO: 5 - protein, 7 aa, synthetic construct
GATGLAA

SEQ ID NO: 6 - protein, 9 aa, synthetic construct
LQSKQYPYT

SEQ ID NO: 7 - protein, 115 aa, synthetic construct
EVQLVESGGGLVQPGGSLRLSCAASGFTFEYFGLQWVRQAPGKGLEWIGYTSSGGNYEYYEETLKGRFTISRDNAKNSLYLQMNSLRAEDTAVYYCARTKTGDYWGQGTLVTVSS

SEQ ID NO: 8 - protein, 107 aa, synthetic construct
DIQMTQSPSSLSASVGDRVTITCRASQDIGNSLTWYQQKPGKAPKRVIFGATGLAAGVPSRFSGSGSGTDFTLTISSLQPEDFATYYCLQSKQYPYTFGQGTKLEIK

SEQ ID NO: 9 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 10 - protein, 17 aa, synthetic construct
DKTHTCPPCPAPEAAGA

SEQ ID NO: 11 - protein, 103 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 12 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 445 aa, synthetic construct
EVQLVESGGGLVQPGGSLRLSCAASGFTFEYFGLQWVRQAPGKGLEWIGYTSSGGNYEYYEETLKGRFTISRDNAKNSLYLQMNSLRAEDTAVYYCARTKTGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 214 aa, synthetic construct
DIQMTQSPSSLSASVGDRVTITCRASQDIGNSLTWYQQKPGKAPKRVIFGATGLAAGVPSRFSGSGSGTDFTLTISSLQPEDFATYYCLQSKQYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 10 aa, synthetic construct
GGSISSYYWS

SEQ ID NO: 16 - protein, 16 aa, synthetic construct
RIYITGNTNYNPSLKD

SEQ ID NO: 17 - protein, 10 aa, synthetic construct
EGPKLGIFDY

SEQ ID NO: 18 - protein, 12 aa, synthetic construct
RASQSVSSSYLA

SEQ ID NO: 19 - protein, 7 aa, synthetic construct
GTESRAT

SEQ ID NO: 20 - protein, 8 aa, synthetic construct
QQYGGYPT

SEQ ID NO: 21 - protein, 118 aa, synthetic construct
QVQLVQSGAEVKKPGSSVKVSCKVSGGSISSYYWSWVRQAPGQGLEWIGRIYITGNTNYNPSLKDRVTMTVDTSTSTHYMELSSLRSEDTAVYYCAREGPKLGIFDYWGQGTLVTVSS

SEQ ID NO: 22 - protein, 107 aa, synthetic construct
DIQMTQSPSSLSASVGDRVTITCRASQSVSSSYLAWYQQKPGKAPKLLIYGTESRATGVPSRFSGSGSGTDFTLTISSLQPEDFATYYCQQYGGYPTFGGGTKVEIK

SEQ ID NO: 23 - protein, 448 aa, synthetic construct
QVQLVQSGAEVKKPGSSVKVSCKVSGGSISSYYWSWVRQAPGQGLEWIGRIYITGNTNYNPSLKDRVTMTVDTSTSTHYMELSSLRSEDTAVYYCAREGPKLGIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 214 aa, synthetic construct
DIQMTQSPSSLSASVGDRVTITCRASQSVSSSYLAWYQQKPGKAPKLLIYGTESRATGVPSRFSGSGSGTDFTLTISSLQPEDFATYYCQQYGGYPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 17 aa, synthetic construct
EKTHTCPPCPAPEAAGA

SEQ ID NO: 26 - protein, 107 aa, synthetic construct
GQPREPQVYTLPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 445 aa, synthetic construct
EVQLVESGGGLVQPGGSLRLSCAASGFTFEYFGLQWVRQAPGKGLEWIGYTSSGGNYEYYEETLKGRFTISRDNAKNSLYLQMNSLRAEDTAVYYCARTKTGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 17 aa, synthetic construct
RKTHTCPPCPAPEAAGA

SEQ ID NO: 29 - protein, 107 aa, synthetic construct
GQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 30 - protein, 448 aa, synthetic construct
QVQLVQSGAEVKKPGSSVKVSCKVSGGSISSYYWSWVRQAPGQGLEWIGRIYITGNTNYNPSLKDRVTMTVDTSTSTHYMELSSLRSEDTAVYYCAREGPKLGIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 31 - DNA, 345 bp, synthetic construct
  source 1..345: mol_type = other DNA
gaggtgcagctggtggagtctgggggaggcttggtccagcctggggggtccctgagactctcctgtgcagcctctggattcacctttgagtactttggattgcagtgggtccgccaggctccagggaaggggctggagtggattggatacaccagtagtggtggaaattacgagtactatgaagaaacgctgaagggccgattcaccatctccagagacaacgccaagaactcactgtatctgcaaatgaacagcctgagagccgaggacacggctgtgtattactgtgcgagaacgaagaccggggactactggggccaaggaaccctggtcaccgtctcctca

SEQ ID NO: 32 - DNA, 321 bp, synthetic construct
  source 1..321: mol_type = other DNA
gacatccagatgacccagtctccatcctccctgtctgcatctgtaggagacagagtcaccatcacttgccgggcaagtcaggacattggaaattctttaacctggtatcagcagaaaccagggaaagcccctaagcgtgtgatctttggtgcaaccggcttggcagctggggtcccatcaaggttcagtggcagtggatctgggacagatttcactctcaccatcagcagtctgcaacctgaagattttgcaacttactactgtctacagagtaaacagtatccttacacttttggccaggggaccaagctggagatcaaa

SEQ ID NO: 33 - DNA, 1335 bp, synthetic construct
  source 1..1335: mol_type = other DNA
gaggtgcagctggtggagtctgggggaggcttggtccagcctggggggtccctgagactctcctgtgcagcctctggattcacctttgagtactttggattgcagtgggtccgccaggctccagggaaggggctggagtggattggatacaccagtagtggtggaaattacgagtactatgaagaaacgctgaagggccgattcaccatctccagagacaacgccaagaactcactgtatctgcaaatgaacagcctgagagccgaggacacggctgtgtattactgtgcgagaacgaagaccggggactactggggccaaggaaccctggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggaaaa

SEQ ID NO: 34 - DNA, 642 bp, synthetic construct
  source 1..642: mol_type = other DNA
gacatccagatgacccagtctccatcctccctgtctgcatctgtaggagacagagtcaccatcacttgccgggcaagtcaggacattggaaattctttaacctggtatcagcagaaaccagggaaagcccctaagcgtgtgatctttggtgcaaccggcttggcagctggggtcccatcaaggttcagtggcagtggatctgggacagatttcactctcaccatcagcagtctgcaacctgaagattttgcaacttactactgtctacagagtaaacagtatccttacacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 35 - DNA, 354 bp, synthetic construct
  source 1..354: mol_type = other DNA
caggtgcagctggtgcagtctggggctgaggtgaagaagcctgggtcctcggtgaaggtctcctgcaaggtctctggaggctccatcagcagctattactggagctgggtgcgacaggcccctggacaagggcttgagtggattggacgtatctatatcaccggtaacaccaactacaacccctccctcaaggacagagtcacgatgaccgtagacacgtccacgagcacacactacatggagctgagcagcctgagatctgaggacacggccgtgtattactgtgcgagagaagggcccaaactggggatctttgactactggggccaaggaaccctggtcaccgtctcctca

SEQ ID NO: 36 - DNA, 321 bp, synthetic construct
  source 1..321: mol_type = other DNA
gacatccagatgacccagtctccatcctccctgtctgcatctgtaggagacagagtcaccatcacttgccgggcaagtcagagtgttagcagcagctatttagcctggtatcagcagaaaccagggaaagcccctaagctcctgatctatggtacagagagtagggccactggggtcccatcaaggttcagtggcagtggatctgggacagatttcactctcaccatcagcagtctgcaacctgaagattttgcaacttactactgtcaacagtatggtggctaccctactttcggcggagggaccaaggtggagatcaaa

SEQ ID NO: 37 - DNA, 1344 bp, synthetic construct
  source 1..1344: mol_type = other DNA
caggtgcagctggtgcagtctggggctgaggtgaagaagcctgggtcctcggtgaaggtctcctgcaaggtctctggaggctccatcagcagctattactggagctgggtgcgacaggcccctggacaagggcttgagtggattggacgtatctatatcaccggtaacaccaactacaacccctccctcaaggacagagtcacgatgaccgtagacacgtccacgagcacacactacatggagctgagcagcctgagatctgaggacacggccgtgtattactgtgcgagagaagggcccaaactggggatctttgactactggggccaaggaaccctggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggaaaa

SEQ ID NO: 38 - DNA, 642 bp, synthetic construct
  source 1..642: mol_type = other DNA
gacatccagatgacccagtctccatcctccctgtctgcatctgtaggagacagagtcaccatcacttgccgggcaagtcagagtgttagcagcagctatttagcctggtatcagcagaaaccagggaaagcccctaagctcctgatctatggtacagagagtagggccactggggtcccatcaaggttcagtggcagtggatctgggacagatttcactctcaccatcagcagtctgcaacctgaagattttgcaacttactactgtcaacagtatggtggctaccctactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 39 - DNA, 1335 bp, synthetic construct
  source 1..1335: mol_type = other DNA
gaggtgcagctggtggagtctgggggaggcttggtccagcctggggggtccctgagactctcctgtgcagcctctggattcacctttgagtactttggattgcagtgggtccgccaggctccagggaaggggctggagtggattggatacaccagtagtggtggaaattacgagtactatgaagaaacgctgaagggccgattcaccatctccagagacaacgccaagaactcactgtatctgcaaatgaacagcctgagagccgaggacacggctgtgtattactgtgcgagaacgaagaccggggactactggggccaaggaaccctggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgagaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaagagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggaaaa

SEQ ID NO: 40 - DNA, 1344 bp, synthetic construct
  source 1..1344: mol_type = other DNA
caggtgcagctggtgcagtctggggctgaggtgaagaagcctgggtcctcggtgaaggtctcctgcaaggtctctggaggctccatcagcagctattactggagctgggtgcgacaggcccctggacaagggcttgagtggattggacgtatctatatcaccggtaacaccaactacaacccctccctcaaggacagagtcacgatgaccgtagacacgtccacgagcacacactacatggagctgagcagcctgagatctgaggacacggccgtgtattactgtgcgagagaagggcccaaactggggatctttgactactggggccaaggaaccctggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtcggaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaggctcaccgtggacaagagcaggtggcagcaggggaacgtcttctcatgctccgtgatgcatgaggctctgcacaaccactacacgcagaagagcctctccctgtccccgggaaaa

SEQ ID NO: 41 - protein, 636 aa, synthetic construct
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 42 - protein, 636 aa, synthetic construct
MRGGRGAPFWPWPLPKLALLPLLWLLFQRTRPQGSAGPLQCYGVGPLGDLNCSWEPLGDLGAPSELHFQSQKYRSNKTQTVAVAAGQSWVAIPREQLTMSDKLLVWGTKAGQPLWTPIFVNLETQMKPNAPQLGPDVDVSEDEPLEAIVHWAPPTWPSHKVLICQFHYRRCQEAAWTLLELELKTIPLAPVEIQDLELATGYQVYGRCRMEKEEDLWGEWSPILSFQTPPAAPKDVWVSGNLCGTPGGEEPLLLWKAPGPCVQVSYKVWFWVGGRELSPEGVTCCGSLIPSGAEWARVSAINATSWEPLTNLSLVCLDSASAPHSVAVSSIAGSTELLVTWRPGPGEPREHVVDWTRDGDPLEKLNWVRLPPGNFSALLPGNFTVGVPYRITVTAVSASGLAPAPSIWGFREELAPLVGPTLWRLQDAPPGTPAIAWGEVPRHQLRGHLTHYTLCAQGGTSPSVCMNVSGNTQSVTLPDLPWGPCELWVTASTIAGQGPPGPILRLHLPDNTLRWKVLPGILFLWGLFLMGCGLSLATSGRCYHLWHKVLPRWVWEKVPDPANSSSGQPHMEQVPEAQPLADLPILEVEEMEPPPVMESSQPPQVTAPLDSGYEKHFLPTPEELGLLGTLRPQVLA

SEQ ID NO: 43 - protein, 631 aa, synthetic construct
MSWLRVARLKPLELLLPLLPLLLGTRPHGSPGPLQCYSVGPLGILNCSWEPLGDLETPPVLYHQSQKYHPNRTKEVKVPSRQSWVTIPRKQFTTADELLIWGTQEGQLLWPPVSVNLETQMKPDTPQIFSDVDFFKEETLEAIVQWEPPKWPLHKVLICQFRYKKCQTKEWTQLEPQLKTDLMTPIEMQNLEPGTGYQVSGRCQVENGYPWGEWSPDLSFPTPFLAPEDVWVSGTICETSGKPAALLFWKDPGPCVQVTYTVWLGVGDVTTTQEEVPCCKSPIPAQIEWAVVSTGNQTSWMPLTNLSLVCLAPESAPCDVRVSSTDGSPGIKVTWKQETGKPWAYVVDWAQDGDSLDKLNWTRLPPENLSTLLSGDFQAGVPYRITVTSVYSGGLAAAPSVWGFIKELAPLAGPAVWRLPDDPTGTPVVAWGEVPRHQLRGQATHYTFCRQSRGIPTICMNVSSQTKTVTLPNLYSGPFKLWVMVSTAAGQGPPGPSLSLHIPDNRIRWKFLPWVLSLWGLLLVGCGLSLAPPRCLYQRHKLLPQWIWERVPDPANSNSGQPYIKEVSLPQPPKDGPILEVEEMELQPVTESPKASVPLYSGYEKHFLPTPEELGLLRPPAPRVLDCSASG

SEQ ID NO: 44 - protein, 623 aa, synthetic construct
MNRLRVARLTPLELLLSLMSLLLGTRPHGSPGPLQCYSVGPLGILNCSWEPLGDLETPPVLYHQSQKYHPNRVWEVKVPSKQSWVTIPREQFTMADKLLIWGTQKGRPLWSSVSVNLETQMKPDTPQIFSQVDISEEATLEATVQWAPPVWPPQKVLICQFRYKECQAETWTRLEPQLKTDGLTPVEMQNLEPGTCYQVSGRCQVENGYPWGEWSSPLSFQTPFLDPEDVWVSGTVCETSGKRAALLVWKDPRPCVQVTYTVWFGAGDITTTQEEVPCCKSPVPAWMEWAVVSPGNSTSWVPPTNLSLVCLAPESAPCDVGVSSADGSPGIKVTWKQGTRKPLEYVVDWAQDGDSLDKLNWTRLPPGNLSTLLPGEFKGGVPYRITVTAVYSGGLAAAPSVWGFREELVPLAGPAVWRLPDDPPGTPVVAWGEVPRHQLRGQATHYTFCIQSRGLSTVCRNVSSQTQTATLPNLHLGSFKLWVTVSTVAGQGPPGPNLSLHLPDNRIRWKALPWFLSLWGLLLMGCGLSLASTRCLQARCLHWRHKLLPQWIWERVPDPANSNSGQPYIKEVSLPQPPKDGPILEVEEVELQPVVESPKASAPIYSGYEKHFLPTPEELGLLV

SEQ ID NO: 45 - protein, 918 aa, synthetic construct
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 46 - protein, 918 aa, synthetic construct
MLTLQTWVVQALFIFLTTESIGELLDPCGYISPESPVVQLHSNFTAVCVLKEKCMDYFHVNANYIVWKTNHFTIPKEQYTIINRTASSVTFTDISSLNIQLTCNILTFGQLEQNVYGITIISGLPPEKPKNLSCIVNEGKKMRCEWNRGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIRLKYNIQYRTKDASTWSQIPPEDTASTRSSFTVQDLKPFTEYVFRICCMKEDGKGYWSDWSEEANGITYEDRPSKAPSFWYKIDPSHAQGYRTVQLMWKTLPPFEANGKILDYEVTLTRWKSHLQNYTVNDTKLTVNLTNDRYVATLTARNLVGKSDAAVLTIPACDFQATHPVMDLKAFPKDNMLWVEWTTPRESVKKYILEWCVLSDKAPCIADWQQEDGTVHRTHLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFSSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGSDDILPRQQYFKQNCSQHESSPDISHFERSKQVSSVNEEDFVRLKQQISDHISQSCGSGEMKMFQEVSAADPFGPGTEGQVERFETIGMEAAIDEGMPKSYLPQTVRQGGYMPQ

SEQ ID NO: 47 - protein, 918 aa, synthetic construct
MSALRIWLMQALLIFLTTESIGQLVEPCGYIYPEFPVVQRGSNFTATCVLKEKCLQVYSVNATYIVWKTNHVAVPKEQVTVINRTASSVTFTDVVFQNVQLTCNILSFGQIEQNVYGITILSGYPPDIPTNLSCIVNEGKNMLCQWDPGRETYLETNYTLKSEWATEKFPDCRTKHGTSSCMMGYTPIYFVNIEVWVEAENALGNVSSEPINFDPVDKVKPSPPHNLSVTNSEELSSILKLAWVNSGLDSILRLKSDIQYRTKDASTWIQVPLEDTVSPRTSFTVQDLKPFTEYVFRIRSIKENGKGYWSDWSEEASGTTYEDRPSKAPSFWYKVNANHPQEYRSARLIWKTLPLSEANGKILDYEVVLTQSKSVSQTYTVNGTELIVNLTNNRYVASLAARNVVGKSPATVLTIPGSHFKASHPVVDLKAFPKDNLLWVEWTPPSKPVNKYILEWCVLSENSPCIPDWQQEDGTVNRTHLRGSLLESKCYLITVTPVFPGGPGSPESMKAYLKQAAPSKGPTVRTKKVGKNEAVLEWDHLPVDVQNGFIRNYSISYRTSVGKEMVVRVDSSHTEYTLSSLSSDTLYMVHMAAYTEEGGKDGPEFTFTTLKFAQGEIEAIVVPVCLAFLLTTLLGVLFCFNKRDLIKKHIWPNVPDPSKSHIAQWSPHTPPRHNFNSKDQMYSDANFTDVSVVEIEANNKKPCPDDLKSLDLFKKEKISTEGHSSGIGGSSCMSSSRPSISSSEENESAQSTASTVQYSTVVHSGYRHQVPSVQVFSRSESTQPLLDSEERPEDLQLVDSVDSGDEILPRQQYFKQSCSQPGASPDVSHFERSSQVPSGSEEDFVRLKQQQVSDHISEPYGSEQRRLFQEGSVADALGTGTDGQIERFESVGMETAMDEDISKSYLPQTVRQGGYMPQ

SEQ ID NO: 48 - protein, 917 aa, synthetic construct
MSAPRIWLAQALLFFLTTESIGQLLEPCGYIYPEFPVVQRGSNFTAICVLKEACLQHYYVNASYIVWKTNHAAVPREQVTVINRTTSSVTFTDVVLPSVQLTCNILSFGQIEQNVYGVTMLSGFPPDKPTNLTCIVNEGKNMLCQWDPGRETYLETNYTLKSEWATEKFPDCQSKHGTSCMVSYMPTYYVNIEVWVEAENALGKVSSESINFDPVDKVKPTPPYNLSVTNSEELSSILKLSWVSSGLGGLLDLKSDIQYRTKDASTWIQVPLEDTMSPRTSFTVQDLKPFTEYVFRIRSIKDSGKGYWSDWSEEASGTTYEDRPSRPPSFWYKTNPSHGQEYRSVRLIWKALPLSEANGKILDYEVILTQSKSVSQTYTVTGTELTVNLTNDRYVASLAARNKVGKSAAAVLTIPSPHVTAAYSVVNLKAFPKDNLLWVEWTPPPKPVSKYILEWCVLSENAPCVEDWQQEDATVNRTHLRGRLLESKCYQITVTPVFATGPGGSESLKAYLKQAAPARGPTVRTKKVGKNEAVLAWDQIPVDDQNGFIRNYSISYRTSVGKEMVVHVDSSHTEYTLSSLSSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANNKKPCPDDLKSVDLFKKEKVSTEGHSSGIGGSSCMSSSRPSISSNEENESAQSTASTVQYSTVVHSGYRHQVPSVQVFSRSESTQPLLDSEERPEDLQLVDSVDGGDEILPRQPYFKQNCSQPEACPEISHFERSNQVLSGNEEDFVRLKQQQVSDHISQPYGSEQRRLFQEGSTADALGTGADGQMERFESVGMETTIDEEIPKSYLPQTVRQGGYMPQ

SEQ ID NO: 49 - protein, 439 aa, synthetic construct
FPRPPGRPQLSLQELRREFTVSLHLARKLLSEVRGQAHRFAESHLPGVNLYLLPLGEQLPDVSLTFQAWRRLSDPERLSFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLECVLSRAVRELLLLSKAGHSVWPLGFPTLSPQPGGGGSTGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVLHEALHSHYTQKSLSLSPKHHHHHH

SEQ ID NO: 50 - protein, 220 aa, synthetic construct
RKGPPAALTLPRVQCRASRYPIAVDCSWTLPPAPNSTSPVSFIATYRLGMAARGHSWPCLQQTPTSTSCTITDVQLFSCAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGSGDYKDDDDK

SEQ ID NO: 51 - protein, 462 aa, synthetic construct
FPRPPGRPQLSLQELRREFTVSLHLARKLLSEVRGQAHRFAESHLPGVNLYLLPLGEQLPDVSLTFQAWRRLSDPERLSFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLECVLSRAVRELLLLSKAGHSVWPLGFPTLSPQPENLYFQGVTGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 52 - protein, 231 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 53 - DNA, 23 bp, synthetic construct
  source 1..23: mol_type = other DNA
gtggtatcaacgcagagtacgcg

SEQ ID NO: 54 - DNA, 27 bp, synthetic construct
  source 1..27: mol_type = other DNA
gaagatgaagacagatggtgcagccac

SEQ ID NO: 55 - DNA, 23 bp, synthetic construct
  source 1..23: mol_type = other DNA
gtggtatcaacgcagagtacgcg

SEQ ID NO: 56 - DNA, 21 bp, synthetic construct
  source 1..21: mol_type = other DNA
gggtgccagggggaagacsga

SEQ ID NO: 57 - protein, 441 aa, synthetic construct
DIQMTQSPSSLSASVGDRVTITCRASQSVSSSYLAWYQQKPGKAPKLLIYGTESRATGVPSRFSGSGSGTDFTLTISSLQPEDFATYYCQQYGGYPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 58 - protein, 221 aa, synthetic construct
QVQLVQSGAEVKKPGSSVKVSCKVSGGSISSYYWSWVRQAPGQGLEWIGRIYITGNTNYNPSLKDRVTMTVDTSTSTHYMELSSLRSEDTAVYYCAREGPKLGIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 59 - protein, 445 aa, synthetic construct
EVQLVESGGGLVQPGGSLRLSCAASGFTFEYFGLQWVRQAPGKGLEWIGYTSSGGNYEYYEETLKGRFTISRDNAKNSLYLQMNSLRAEDTAVYYCARTKTGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 154 aa, synthetic construct
RFSGSGSGTDFTLTISSLQPEDFATYYCLQSKQYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0052047 A1
(43) Pub. Date: Feb. 15, 2024

(54) ANTI- IL27R ANTIBODIES AND METHODS OF USE THEREOF

(71) Applicant: Pfizer Inc., New York, NY (US)

(72) Inventors: Yijie GAO, Weston, MA (US); Nicholas Andrew MARZE, Quincy, MA (US); Laird BLOOM, Needham, MA (US)

(21) Appl. No.: 18/363,152

(22) Filed: Aug. 1, 2023

Related U.S. Application Data
(60) Provisional application No. 63/511,276, filed on Jun. 30, 2023.
(60) Provisional application No. 63/370,306, filed on Aug. 3, 2022.

(51) Int. Cl. C07K 16/28

(52) U.S. Cl. CPC C07K 16/2866; C07K 16/2803; A61K 2039/505

(57) ABSTRACT

The present invention relates to antibodies that specifically bind to one or both of IL27RA and gp130. The present invention further relates to bispecific antibodies that specifically bind to IL27RA and gp130. The present invention also pertains to related molecules, e.g. nucleic acids which encode such antibodies or bispecific antibodies, compositions, and related methods, e.g., methods for producing and purifying such antibodies and bispecific antibodies, and their use in diagnostics and therapeutics

### REFERENCE TO SEQUENCE LISTING

The instant application contains a Sequence Listing which has been submitted electronically in .xml format and is hereby incorporated by reference in its entirety. Said .xml copy, created on Jun. 30, 2023, is named PC072877 Sequence Listing ST26.xml and is 72,523 bytes in size.

### BACKGROUND

The present invention relates to antibodies that specifically bind to one or both of IL27RA and gp130. The present invention further relates to bispecific antibodies that specifically bind to IL27RA and gp130. The present invention also pertains to related molecules, e.g. nucleic acids which encode such antibodies or bispecific antibodies, compositions, and related methods, e.g., methods for producing and purifying such antibodies and bispecific antibodies, and their use in diagnostics and therapeutics.

Inflammatory bowel disease (IBD), including Crohn's disease (CD) and ulcerative colitis (UC), refers to a collection of idiopathic chronic inflammatory disorders of the intestine. Crohn's disease involves the ileum and colon, but it can affect any region of the intestine, often discontinuously. Ulcerative colitis involves the rectum, part of the colon or the entire colon (pancolitis) in an uninterrupted pattern. The pathogenesis of IBD remains unclear, but is thought to be multifactorial, including genetic and environmental components, among which abnormal immune responses to commensal bacteria and/or food antigens may be the central part. Therefore, the development of highly effective therapeutic methods for patients to regulate excess immune responses is an important, unmet need Hazel K and O'Connor A. Emerging treatments for inflammatory bowel disease. Therapeutic Advances in Chronic Disease. 2020, Vol. 11: 1-12.

Interleukin (IL)-27 is a heterodimeric cytokine in the IL-12 cytokine family. It is composed of two subunits: Epstein-Barr virus-induced gene 3 (EBi3) and IL-27p28. IL-27 plays an immune modulatory role after binding to its heterodimeric receptor, which contains an IL-27 selective subunit, IL27RA, and a subunit common to multiple signaling receptors, glycoprotein 130 (gp130). The co-expression of the two receptor subunits has been verified on T cells, monocytes, macrophages, dendritic cells, colon epithelial cells, keratinocytes, etc. The engaging of the two receptor subunits directly activates JAK1, JAK2, and Tyk2 kinases, and induces tyrosine phosphorylation of signal transducers and activators of transcription (STAT), which form homodimers or heterodimers and translocate into the nucleus to modulate gene expression. In addition to the Jak/STAT signaling pathway, IL-27 has been reported to induce p38MAPK, ERK, and Akt signaling under certain cellular environments Hunter C A and Kastelein R. Fifteen years of interleukin-27-discovery, advances and translation. Immunity. 2012 Dec. 14; 37(6): 960-969.

IL-27 has been implicated as a candidate for IBD treatment in multiple studies. A genome-wide association study in early-onset IBD identified IL-27 within a susceptibility locus in a North American-European cohort. In support of that conclusion, the authors also demonstrated that healthy individuals with two copies of the risk allele expressed significantly less IL-27 relative to individuals with two copies of the non-risk allele and that IL-27 colonic gene expression was significantly lower in samples obtained from individuals with early-onset CD and UC cases than in normal tissue. Imielinski M, Baldassano R N, and Griffiths A, et. al., Common variants at five new loci associated with early-onset inflammatory bowel disease. Nat Genet. 2009 December; 41(12): 1335-1340. IL-27 polymorphisms have also been associated with risk for IBD in both Chinese and Korean populations. Wang Z, Wang L, Fan R, et al. Association of IL-27 gene three polymorphisms with Crohn's disease susceptibility in a Chinese Han population. Int J Clin Exp Pathol. 2014; 7(12):8952-8957 and Li C S, Zhang Q, Lee K J, et al. Interleukin-27 polymorphisms are associated with inflammatory bowel diseases in a Korean population. J Gastroenterol Hepatol. 2009; 24(10):1692-1696.

IL-27 has been shown to ameliorate colitis in mouse models, through lessening of induced colonic inflammation by IL-27 administration and by directly promoting intestinal epithelial barrier function via transcriptional activation of anti-inflammatory and anti-bacterial genes, and conversely, through the demonstration of more severe colitis in mice deficient in IL-27Ra due to genetic knockout. Hanson M L, Hixon J A, and Li W, et. al., Oral Delivery of IL-27 Recombinant Bacteria Attenuates Immune Colitis in Mice. Gastroenterology 2014; 146:210-221; Troy A E, Zaph C, and Du Y, et. al., IL-27 Regulates Homeostasis of the Intestinal CD4_Effector T Cell Pool and Limits Intestinal Inflammation in a Murine Model of Colitis. JI, 2009, 183: 2037-2044; Diegelmann J, Olszak T, and Goke B, et. al., c. A Novel Role for Interleukin-27 (IL-27) as Mediator of Intestinal Epithelial Barrier Protection Mediated via Differential Signal Transducer and Activator of Transcription (STAT) Protein Signaling and Induction of Antibacterial and Anti-inflammatory Proteins. JBC. 2012, 287(1), pp. 286-298. More specifically, mucosal administration of an IL-27-expressing food-grade bacterium Lactococcus lactis (LL-IL-27) or subcutaneous treatment with IL-27 have been shown to protect mice from enterocolitis and death in T-cell transfer—induced colitis and 2,4,6-trinitrobenzenesulfonic acid (TNBS) induced colitis models. Hanson M L, Hixon J A, and Li W, et. al., Oral Delivery of IL-27 Recombinant Bacteria Attenuates Immune Colitis in Mice. Gastroenterology 2014; 146:210-221; Sasaoka T, Ito M, and Yamashita J, et al., Treatment with IL-27 attenuates experimental colitis through the suppression of the development of IL-17-producing T helper cells. Am J Physiol Gastrointest Liver Physiol 2011, 300: G568-G576; Andrews C, McLean M H, and Durum S K. IL-27 as a novel therapy for inflammatory bowel disease: a critical review of the literature. Inflamm Bowel Dis. 2016 September; 22(9): 2255-2264.

The role of IL-27 as a potential therapeutic approach has been suggested for the variety of auto-immune conditions besides IBD, including asthma and allergic diseases. [11, 12] Metabolic disorders such as obesity and type 2 diabetes would also be attractive therapeutic areas to consider where IL-27 agonism could be beneficial.

There is a long-felt unmet need for novel therapeutics to treat or ameliorate IBD, including UC and CD, as well as to treat other autoimmune conditions. Despite IL27R being implicated as a target for the treatment of IBD, including UC and CD, there are currently no effective treatments targeting IL27R. The present invention meets these needs.

### SUMMARY

Provided herein are antibodies (including antigen binding fragments thereof) that bind to one or more or interleukin receptor subunit alpha (IL27RA) and glycoprotein 130 (gp130) including bispecific antibodies that specifically bind to IL27RA and gp130 and other related antibodies, as well as uses of the antibodies and associated methods.

The disclosure also provides processes for making, preparing, and producing antibodies that bind to one or more or IL27RA and gp130 including bispecific antibodies that specifically bind to IL27RA and gp130. Antibodies of the disclosure are useful in one or more of diagnosis, prophylaxis, or treatment of disorders or conditions mediated by, or associated with, IL27 activity, including, but not limited to inflammatory bowel disease (IBD), Crohn's disease (CD). ulcerative colitis (UC), multiple sclerosis, rheumatoid arthritis, celiac disease, asthma, allergic diseases, obesity, type 2 diabetes and cancer.

The disclosure further encompasses expression of antibodies, and preparation and manufacture of compositions comprising antibodies of the disclosure, such as medicaments for the use of the antibodies.

Polynucleotides encoding antibodies that bind to one or more of IL27RA and gp130 including bispecific antibodies that specifically bind to IL27RA and gp130. Polynucleotides encoding antibody heavy chains or light chains, or both are also provided. Host cells that express the antibodies are provided. Methods of treatment using the antibodies are provided. Such methods include, but are not limited to, one or more of methods of treating or methods of preventing diseases associated with or mediated by IL27 expression and or binding to the IL27 receptor. Diseases associated with or mediated by IL27 expression and or binding to the IL27 receptor include inflammatory bowel disease (IBD), multiple sclerosis, rheumatoid arthritis, celiac disease, asthma, allergic diseases, obesity, type 2 diabetes and cancer.

In some embodiments, there is provided an isolated antibody that specifically binds to IL27RA, comprising a heavy chain variable region (IL27RA-VH) and a light chain variable region (IL27RA-VL), comprising the CDR-H1, CDR-H2, and CDR-H3 sequences of SEQ ID NO: 7, and the CDR-L1, CDR-L2, and CDR-L3 sequences of SEQ ID NO: 8.

In some embodiments, there is provided an isolated antibody that specifically binds to IL27RA, comprising a heavy chain variable region (IL27RA-VH) and a light chain variable region (IL27RA-VL), comprising a CDR-H1 sequence according to SEQ ID NO: 1; a CDR-H2 sequence according to SEQ ID NO: 2; a CDR-H3 sequence according to SEQ ID NO: 3 and comprising a CDR-L1 sequence according to SEQ ID NO: 4; a CDR-L2 sequence according to SEQ ID NO: 5, and a CDR-L3 sequence according to SEQ ID NO: 6.

In some embodiments, there is provided an isolated antibody comprising an IL27RA-VH sequence encoded by a nucleic acid sequence of SEQ ID NO: 31 and comprising a, IL27RA-VL sequence encoded by a nucleic acid sequence of SEQ ID NO 32.

In some embodiments there is provided an isolated antibody that specifically binds to IL27RA comprising a heavy chain having the amino acid sequence of SEQ ID NO: 13, and a light chain having the amino acid sequence of SEQ ID NO: 14. In some embodiments there is provided an isolated antibody that specifically binds to IL27RA comprising a heavy chain having the amino acid sequence of SEQ ID NO: 27, and a light chain having the amino acid sequence of SEQ ID: 14.

In some embodiments, there is provided an isolated antibody that that competes for binding to IL27RA with a second antibody comprising a VH having the amino acid sequence of SEQ ID NO: 7, and a VL having the amino acid sequence of SEQ ID NO: 8.

In some embodiments, there is provided an isolated polynucleotide encoding the VH of an antibody that binds IL27RA, wherein the polynucleotide comprises the nucleic acid sequence of SEQ ID NO 31.

In some embodiments, there is provided an isolated polynucleotide encoding the VL of an antibody that binds IL27RA, wherein the polynucleotide comprises the nucleic acid sequence of SEQ ID NO: 32.

In some embodiments, there is provided an isolated polynucleotide encoding the VH and VL of an antibody that binds IL27RA wherein the polynucleotide encoding the VH comprises the nucleic acid sequence of SEQ ID NO: 31 and the polynucleotide encoding the VL comprises the nucleic acid sequence of SEQ ID NO: 32.

In some embodiments, there is provided an isolated antibody that specifically binds to glycoprotein 130 (gp130), comprising a heavy chain variable region (gp130-VH) and a light chain variable region (gp130-VL), comprising the CDR-H1, CDR-H2, and CDR-H3 sequences of SEQ ID NO: 21, and the CDR-L1, CDR-L2, and CDR-L3 sequences of SEQ ID NO: 22;

In some embodiments, there is provided an isolated antibody that specifically binds to gp130, comprising a heavy chain variable region (gp130-VH) and a light chain variable region (gp130-VL), comprising a CDR-H1 sequence according to SEQ ID NO: 15; a CDR-H2 sequence according to SEQ ID NO: 16; a CDR-H3 sequence according to SEQ ID NO: 17 and comprising a CDR-L1 sequence according to SEQ ID NO: 18; a CDR-L2 sequence according to SEQ ID NO: 19, and a CDR-L3 sequence according to SEQ ID NO: 20.

In some embodiments, there is provided an isolated antibody that specifically binds to gp130 comprising a heavy chain having the amino acid sequence of SEQ ID NO: 23, and a light chain having the amino acid sequence of SEQ ID NO: 24. In some embodiments, there is provided an isolated antibody that specifically binds to gp130 comprising a heavy chain having the amino acid sequence of SEQ ID NO: 30, and a light chain having the amino acid sequence of SEQ ID NO: 24.

In some embodiments there is provided an isolated antibody that that competes for binding to gp130 with a second antibody comprising a VH having the amino acid sequence of SEQ ID NO: 21, and a VL having the amino acid sequence of SEQ ID NO: 22 In some embodiments there is provided an isolated polynucleotide encoding the VH of an antibody that binds gp130, wherein the polynucleotide comprises the nucleic acid sequence of SEQ ID NO: 35.

In some embodiments there is provided an isolated polynucleotide encoding the VL of an antibody that binds gp130, wherein the polynucleotide comprises the nucleic acid sequence of SEQ ID NO: 36.

In some embodiments there is provided an isolated polynucleotide encoding the VH and VL of an antibody that binds gp130, wherein the polynucleotide encoding the VH comprises the nucleic acid sequence of SEQ ID NO: 35 and the polynucleotide encoding the VL comprises the nucleic acid sequence of SEQ ID NO: 36.

In some embodiments there is provided an isolated polynucleotide encoding the heavy chain, light chain, or both, of an antibody that binds gp130, and wherein said polynucleotide comprises: the nucleic acid sequence of SEQ ID NO: 37, the nucleic acid sequence of SEQ ID NO: 38, or both.

In some embodiments there is provided an isolated polynucleotide encoding the heavy chain, light chain, or both, of an antibody that binds gp130, and wherein said nucleic acid comprises: the nucleic acid sequence of SEQ ID NO: 40, the nucleic acid sequence of SEQ ID NO: 38, or both.

In some embodiments there is provided an isolated antibody comprising a first antigen binding site that binds to IL27RA and a second antigen binding site that binds to gp130, wherein the first antigen binding site comprises a VH and a VL, wherein the second antigen binding site comprises a VH and a VL, and wherein one or both of:
a. the first antigen binding site VH comprises (i) a VH CDR1 comprising the amino acid sequence of SEQ ID NO: 1 (ii) a VH CDR2 comprising the amino acid sequence of SEQ ID NO: 2; and (iii) a VH CDR3 comprising the amino acid sequence of SEQ ID NO: 3;
and b. the first antigen binding site VL comprises (i) a VL CDR1 comprising the amino acid sequence of SEQ ID NO: 4; (ii) a VL CDR2 comprising the amino acid sequence of SEQ ID NO: 5; and (iii) a VL CDR3 comprising the amino acid sequence of SEQ ID NO: 6.

In some embodiments there is provided an isolated antibody comprising a first antigen binding site that binds to IL27RA and a second antigen binding site that binds to gp130, wherein the first antigen binding site comprises a VH and a VL, wherein the second antigen binding site comprises a VH and a VL, and wherein one or both of:
a. the second antigen binding site VH comprises (i) a VH CDR1 comprising the amino acid sequence of SEQ ID NO: 15; (ii) a VH CDR2 comprising the amino acid sequence of SEQ ID NO: 16; and (iii) a VH CDR3 comprising the amino acid sequence of SEQ ID NO: 17; and
b. the second antigen binding site VL comprises (i) a VL CDR1 comprising the amino acid sequence of SEQ ID NO: 18; (ii) a VL CDR2 comprising the amino acid sequence of SEQ ID NO: 19; and (iii) a VL CDR3 comprising the amino acid sequence of SEQ ID NO: 20.

In some embodiments there is provided an isolated antibody comprising a first antigen binding site that binds to IL27RA and a second antigen binding site that binds to gp130, wherein the first antigen binding site comprises a VH and a VL, wherein the second antigen binding site comprises a VH and a VL, and wherein:
a. the first antigen binding site VH comprises (i) a VH CDR1 comprising the amino acid sequence of SEQ ID NO: 1; (ii) a VH CDR2 comprising the amino acid sequence of SEQ ID NO: 2; and (iii) a VH CDR3 comprising the amino acid sequence of SEQ ID NO: 3;
b. the first antigen binding site VL comprises (i) a VL CDR1 comprising the amino acid sequence of SEQ ID NO: 4; (ii) a VL CDR2 comprising the amino acid sequence of SEQ ID NO: 5; and (iii) a VL CDR3 comprising the amino acid sequence of SEQ ID NO: 6;
c. the second antigen binding site VH comprises (i) a VH CDR1 comprising the amino acid sequence of SEQ ID NO: 15; (ii) a VH CDR2 comprising the amino acid sequence of SEQ ID NO: 16; and (iii) a VH CDR3 comprising the amino acid sequence of SEQ ID NO: 17; and
d. the second antigen binding site VL comprises (i) a VL CDR1 comprising the amino acid sequence of SEQ ID NO: 18; (ii) a VL CDR2 comprising the amino acid sequence of SEQ ID NO: 19; and (iii) a VL CDR3 comprising the amino acid sequence of SEQ ID NO: 20.

In some embodiments there is provided an isolated antibody comprising a first antigen binding site that binds to IL27RA and a second antigen binding site that binds to gp130, wherein the antibody comprises a first antigen binding site VH comprising the amino acid sequence of SEQ ID NO: 7, a first antigen binding site VL comprising the amino acid sequence of SEQ ID NO: 8, a second antigen binding site VH comprising the amino acid sequence of SEQ ID NO: 21, and a second antigen binding site VL comprising the amino acid sequence of SEQ ID NO: 22.

In some embodiments there is provided an antibody that binds to both IL27RA and gp130, comprising a first heavy chain and a first light chain, and a second heavy chain and second light chain, wherein the first heavy chain and the first light chain comprise a first antigen binding site that binds to IL27RA, and the second heavy chain and the second light chain comprise a second antigen binding site that binds to gp130, wherein the first antibody heavy chain comprises the amino acid sequence of SEQ ID NO:27, the first antibody light chain comprises the amino acid sequence of SEQ ID NO: 14, the second antibody heavy chain comprises the amino acid sequence of SEQ ID NO: 30, and the second antibody light chain comprises the amino acid sequence of SEQ ID NO: 34.

### BRIEF DESCRIPTION OF THE DRAWINGS

FIG. 1 Schematic of a bispecific anti-IL27RA/gp130 antibody of the invention. Fc heterodimerization is driven through mutations engineered in the CH3 domain.

FIG. 2 Co-crystal structure of parental anti-IL27RA clone 2255 Fab fragment in complex with human IL27RA extracellular domain recombinant protein is available at 3.2 Å resolution. Protein components expressed from HEK293 FIG. 3 Co-crystal structure of a humanized version (3754) of parental anti-gp130 clone 2246 Fab fragment in complex with human gp130 extracellular domain recombinant protein is available at 2.7 Å resolution. Protein components expressed from HEK293.

FIG. 4 shows that there is no effect of IL-27R agonists on IFNγ production in Th1 cells post differentiation.

FIG. 5 The anti-IL27RA/gp130 antibody of the invention increased CD4+CD25+FoxP3+iTreg population and Increased LAG-3 and Tim-3 surface expression.

FIG. 6. Diagram of the mono-Fc version of IL27 ligand complex. CH23LS-Fc: human IgG1 Fc with mutations to stabilize monomeric Fc; Flag: Flag tag; H6: His tag; p28 (f29-p243)-C107-L212C: p28 subunit of IL27 ligand, amino acid sequence F29 to P243 with mutations at C107 to S and L212 to C to allow stabilizing disulfide bond formation between L212 in p28 and M99 in Ebi3; Ebi3 (R21-K229)-M99C: Ebi3 subunit of IL27 ligand amino acid R21 to K229 with M99 to C mutation to allow stabilizing disulfide bond formation with L212C in p28 subunit. CID1613 & 1617: construct numbering.

FIG. 7 Diagram of the Knob and Hole version of IL27 ligand complex. hulgG1Fc “knob”: human IgG1 Fc with “knob” mutations; hulgG1Fc “hole”: human IgG1 Fc with “hole” mutations; Flag: Flag tag; H6: His tag; p28 (f29-p243)-C107-L212C: p28 subunit of IL27 ligand, amino acid sequence F29 to P243, mutations at C107 to S and L212 to C allow stabilizing disulfide bond formation between L212 in p28 with M99 in Ebi3; Ebi3 (R21-K229)-M99C: Ebi3 subunit of IL27 ligand starting at amino acid R21 ends at K229, with M99 to C mutation to allow stabilizing disulfide bond formation with L212C in p28 subunit. CID1353, 1643 & 1617: construct numbering.

FIG. 8. Octet Competition Assay of 2255 vs IL27 Ligand to IL27RA. The sensorgrams were aligned at the end of the 1st association step (˜1059s). The sensorgrams from 1060 seconds evaluate whether GBT-IL-27R-2255 is able to bind hIL27R-CH23Fc-Flag in the presence of hIL27:EBi3. (middle line on the right panel) has hIL27:EBi3 bound to hIL27R and GBT-IL-27R-2255 did not show any binding. This demonstrated that the IL27Ra lead IgG GBT-IL-27R-2255 competes with IL-27 ligand in binding IL27R. Sensor C6 (top line on the right panel) only has hIL27R on the sensor without hIL27LEBi3 showed binding to GBT-IL-27R-2255 in this assay format. Sensor D6 (bottom line on the right panel) was the buffer control. Sensor D6 did not overlay with B6 potentially due to dissociation of hIL27:EBi3 on Sensor D6 when dipped into buffer.

FIG. 9: Schematic view of two bispecific formats. Left: EE/RR format (GBT-IL27R-4894). Right: knob-in-hole mFd format (GBT-IL27R-4933). IL-27RA and gp130-binding Fabs and their VH, VL, CH1, and CL components are labeled. Features used for heterodimerization (E and R mutations or knob and hole) are indicated.

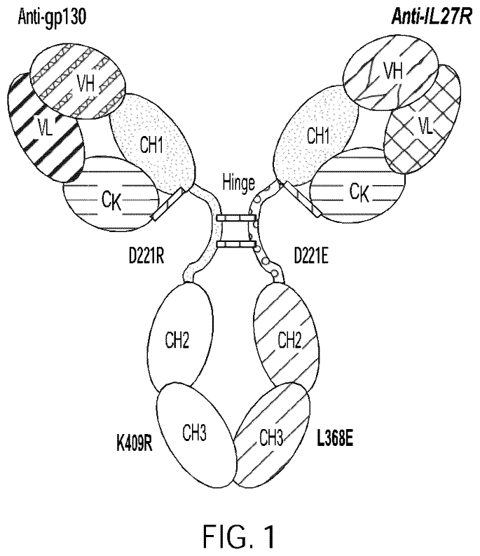

### DETAILED DESCRIPTION

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Exemplary embodiments (E) of the invention provided herein include:
E1. An isolated antibody that specifically binds to IL27RA, comprising a heavy chain variable region (IL27RA-VH) and a light chain variable region (IL27RA-VL), comprising the CDR-H1, CDR-H2, and CDR-H3 sequences of SEQ ID NO: 7, and the CDR-L1, CDR-L2, and CDR-L3 sequences of SEQ ID NO: 8;
E2. An isolated antibody that specifically binds to IL27RA, comprising a heavy chain variable region (IL27RA-VH) and a light chain variable region (IL27RA-VL), comprising a CDR-H1 sequence according to SEQ ID NO: 1; a CDR-H2 sequence according to SEQ ID NO: 2; a CDR-H3 sequence according to SEQ ID NO: 3 and comprising a CDR-L1 sequence according to SEQ ID NO: 4; a CDR-L2 sequence according to SEQ ID NO: 5, and a CDR-L3 sequence according to SEQ ID NO: 6.
E3. The antibody thereof of E1 or E2, comprising an IL27RA-VH framework sequence derived from a human germline VH sequence selected from the group consisting of DP7, DP10, DP35, DP47, DP50, DP51, DP54, and DP77.
E4. The antibody of E1 or E2, comprising an IL27RA-VH framework sequence derived from a human germline DP54 sequence.
E5. The antibody of any one of any one of E1 to E4, comprising a IL27RA-VL framework sequence derived from a human germline VL sequence selected from the group consisting of DPK1, DPK3, DPK4, DPK5, DPK7, DPK8, and DPK9.
E5. The antibody of any one of any one of E1 to E4, comprising a IL27RA-VL framework sequence derived from a human germline DPK9, sequence.
E6. The antibody of any one of E1-E5, comprising a IL27RA-VL framework sequence and a IL27RA-VH framework sequence wherein one or both of the IL27RA-VL framework sequence and the IL27RA-VH framework sequence is at least 90%, 91%, 92%, 93%, 94%, 95%, 96%, 97%, 98%, 99%, or 100% identical to the human germline sequence from which it was derived.
E7. The antibody of any one of E1-E6, comprising a IL27RA-VL framework sequence and a IL27RA-VH framework sequence, and wherein one or both of the IL27RA-VL framework sequence or the IL27RA-VH framework sequence is identical to the human germline sequence from which it was derived.
E8. The antibody of any one of E1-E7, comprising a IL27RA-VH sequence at least 90%, 91%, 92%, 93%, 94%, 95%, 96%, 97%, 98%, 99%, or 100% identical to SEQ ID NO: 7, and comprising a IL27RA-VL sequence at least 90%, 91%, 92%, 93%, 94%, 95%, 96%, 97%, 98%, 99%, or 100% identical to SEQ ID NO: 8.
E9. The antibody of any one of E1-E8, comprising a IL27RA-VH sequence of SEQ ID NO: 7, and comprising a IL27RA-VL sequence of SEQ ID NO: 8.
E10. The antibody of any one of E1-E9, comprising a IL27RA-VH sequence encoded by a polynucleotide sequence of SEQ ID NO: 31.
E11. The antibody of any one of E1-E10, comprising a IL27RA-VL sequence encoded by a nucleic acid sequence of SEQ ID NO 32.
E12 An isolated antibody comprising an IL27RA-VH sequence encoded by a nucleic acid sequence of SEQ ID NO: 31 and comprising a, IL27RA-VL sequence encoded by a nucleic acid sequence of SEQ ID NO 32.
E13. The antibody of any one of E1-E12, further comprising an Fc domain, wherein the Fc domain is an isotype of IgA (for example IgA₁ or IgA₂), IgD, IgE, IgM, or IgG (for example IgG₁, IgG₂, IgG₃, or IgG₄.).
E14. The antibody of any one of E1-E13, comprising a Fc domain of the isotype of IgG.
E15. The antibody of any one of E1-E14, comprising a Fc domain of an isotype of IgG₁.
E16. The antibody of any one of E13-E15, wherein the Fc domain is of a human IgG1 comprising one or more substitutions selected from the group consisting of L234A, L235A, and G237A, by EU numbering, (said group may also be referred to as L247A, L248A, and G250A by Kabat numbering), wherein the numbering is according to human IgG1 wildtype.
E17. The antibody of E16, wherein the antibody comprises an Fc domain comprising the substitutions L234A, L235A and G237A (by EU numbering) or L247A, L248A and G250A (by Kabat numbering) wherein the numbering is according to human IgG1 wildtype.
E18. The antibody thereof of any one of E1 to E17 wherein the antibody comprises an Fc domain comprising the substitutions D221E & L368E (by EU numbering) or D234E & L381E (by Kabat numbering); wherein the numbering is according to human IgG1 wildtype.
E19. The antibody of any one of E1 to E17 wherein the antibody comprises an Fc domain comprising the substitutions D221R & K409R (by EU numbering) or D234R & K422R (by Kabat numbering) wherein the numbering is according to human IgG1 wildtype.
E20. The antibody of any one of E1-E19, comprising a heavy chain having the amino acid sequence of SEQ ID NO: 13 or SEQ ID NO 27.
E21. The antibody of any one of E1-E20, comprising a heavy chain having the amino acid sequence of SEQ ID NO: 13.
E22. The antibody of any one of E1-E20, comprising a heavy chain having the amino acid sequence of SEQ ID NO: 27.
E23. The antibody of any one of E1-E22, comprising a light chain having the amino acid sequence of SEQ ID NO: 14.
E24. The antibody of any one of E1-E23, comprising a heavy chain having the amino acid sequence of SEQ ID NO: 27, and a light chain having the amino acid sequence of SEQ ID NO: 14.
E25. The antibody of any one of E1-E24, wherein the antibody antagonizes IL27RA.
E26. The antibody of any one of E1-E25, wherein the antibody agonizes IL27RA.
E27. The antibody of any one of E1-E25, wherein the antibody binds cynomolgus IL27RA.
E28. The antibody of E1-E27, wherein the binding KD of the antibody cynomolgus IL27RA is within 10 orders of magnitude of the binding KD of the antibody to human IL27RA as measured by SPR.
E29. An isolated antibody that that competes for binding to IL27RA with a second antibody comprising a VH having the amino acid sequence of SEQ ID NO: 7, and a VL having the amino acid sequence of SEQ ID NO: 8.
E30. A pharmaceutical composition comprising a therapeutically effective amount of the antibody of any one of E1-E29 and a pharmaceutically acceptable carrier.
E31 An isolated polynucleotide encoding the antibody of any one of E1 to E29
E32. The polynucleotide of E31, wherein said polynucleotide is RNA.
E33. The polynucleotide of E32, wherein said polynucleotide comprises at least one chemical modification.
E34. The polynucleotide of E33, wherein the chemical modification wherein is selected from pseudouridine, 1-methylpseudouridine. N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine,), 5-methoxyuridine and 2′-O-methyl uridine.
E35. The polynucleotide of E31, wherein said polynucleotide does not comprise a chemical modification.
E35. An isolated polynucleotide encoding the VH of an antibody that binds IL27RA, wherein the polynucleotide comprises the nucleic acid sequence of SEQ ID NO 31.
E36. An isolated polynucleotide encoding the VL of an antibody that binds IL27RA, wherein the polynucleotide comprises the nucleic acid sequence of SEQ ID NO: 32.
E37. An isolated polynucleotide encoding the VH and VL of an antibody that binds IL27RA wherein the polynucleotide encoding the VH comprises the nucleic acid sequence of SEQ ID NO: 31 and the polynucleotide encoding the VL comprises the nucleic acid sequence of SEQ ID NO: 32.
E38. An isolated polynucleotide encoding the heavy chain, light chain, or both, of an antibody that binds IL27RA, and wherein said nucleic acid comprises: the nucleic acid sequence of SEQ ID NO: 33 the nucleic acid sequence of SEQ ID NO: 34, or both.
E39. An isolated polynucleotide encoding the heavy chain, light chain, or both, of an antibody that binds IL27RA, and wherein said nucleic acid comprises: the nucleic acid sequence of SEQ ID NO: 39 the nucleic acid sequence of SEQ ID NO: 34, or both.
E40. A vector comprising the polynucleotide of any one of E31-E39.
E41. An isolated host cell comprising the polynucleotide of any one of E31-E39, or the vector of E40.
E42. A method of producing an isolated antibody, comprising culturing the host cell of E41 under conditions that result in production of the antibody, and recovering the antibody.
E43. The antibody of any one of E1-E29, or the pharmaceutical composition of E30, for use as a medicament.
E44. The antibody of any one of E1-E29, or the pharmaceutical composition of E30, for use in the treatment of an inflammatory disease.
E45. The antibody of any one of E1-E29, or the pharmaceutical composition of E30, for use in the treatment of one or more selected from the group consisting of inflammatory bowel disease (IBD), Crohn's disease (CD), ulcerative colitis (UC), multiple sclerosis, rheumatoid arthritis, celiac disease, asthma, allergic diseases, obesity, type 2 diabetes and cancer.
E46. The antibody of any one of E1-E29, or the pharmaceutical composition of E30, for use in the treatment of Inflammatory bowel disease (IBD).
E47. The antibody of any one of E1-E29, or the pharmaceutical composition of E30, for the use according to E46, wherein the use is for the treatment of Crohn's disease (CD) or ulcerative colitis (UC).
E48. A method of treating a medical condition, comprising administering to a subject in need thereof a therapeutically effective amount of the antibody of any one of E1-E29, or the pharmaceutical composition of E30.
E49. The method of E48, wherein the condition is selected from the group consisting of inflammatory bowel disease (IBD), Crohn's disease (CD), ulcerative colitis (UC), multiple sclerosis, rheumatoid arthritis, celiac disease, asthma, allergic diseases, obesity, type 2 diabetes and cancer.
E50. The method of E48, wherein the condition is an inflammatory disease.
E51. The method of E48, wherein the condition is Inflammatory bowel disease (IBD).
E52. The method of any one of E48 to E51, or the use of E43 to E47 comprising administering said antibody, or pharmaceutical composition, subcutaneously.
E53. The method of any one of E48 to E51, or the use of E43 to E47 wherein said antibody, or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.
E54. The use of the antibody of any one of E1-E29 for the manufacture of a medicament for use in the treatment of an inflammatory bowel disease (IBD), Crohn's disease (CD), ulcerative colitis (UC), multiple sclerosis, rheumatoid arthritis, celiac disease, asthma, allergic diseases, obesity, type 2 diabetes and cancer.
E55. The use of the antibody of any one of E1-E29 for the manufacture of a medicament for use in the treatment of an inflammatory disease.
E56. The use of the antibody according to E54 or E55, wherein the condition is inflammatory bowel disease (IBD).
E57. An isolated antibody that specifically binds to glycoprotein 130 (gp130), comprising a heavy chain variable region (gp130-VH) and a light chain variable region (gp130-VL), comprising the CDR-H1, CDR-H2, and CDR-H3 sequences of SEQ ID NO: 21, and the CDR-L1, CDR-L2, and CDR-L3 sequences of SEQ ID NO: 22;
E58. An isolated antibody that specifically binds to gp130, comprising a heavy chain variable region (gp130-VH) and a light chain variable region (gp130-VL), comprising a CDR-H1 sequence according to SEQ ID NO: 15; a CDR-H2 sequence according to SEQ ID NO: 16; a CDR-H3 sequence according to SEQ ID NO: 17 and comprising a CDR-L1 sequence according to SEQ ID NO: 18; a CDR-L2 sequence according to SEQ ID NO: 19, and a CDR-L3 sequence according to SEQ ID NO: 20.
E59 The antibody of E57 or E58, comprising a gp130-VH framework sequence derived from a human germline VH sequence selected from the group consisting of DP7, DP10, DP35, DP47, DP50, DP51, DP54, and DP77
E60. The antibody of any one of E57 to E59, comprising a gp130-VH framework sequence derived from a human germline DP10, sequence.
E61. The antibody of any one of any one of E57 to E60, comprising a gp130-VL framework sequence derived from a human germline VL sequence selected from the group consisting of DPK1, DPK3, DPK4, DPK5, DPK7, DPK8, and DPK9
E62. The antibody of any one of any one of E57 to E61, comprising a gp130-VL framework sequence derived from a human germline DPK9, sequence.
E63. The antibody of any one of E57-E62, comprising a gp130-VL framework sequence and a gp130-VH framework sequence wherein one or both of the gp130-VL framework sequence and the gp130-VH framework sequence is at least 90%, 91%, 92%, 93%, 94%, 95%, 96%, 97%, 98%, 99%, or 100% identical to the human germline sequence from which it was derived.
E64. The antibody of any one of E57-E63, comprising a gp130-VL framework sequence and a gp130-VH framework sequence, and wherein one or both of the gp130-VL framework sequence or the gp130-VH framework sequence is identical to the human germline sequence from which it was derived.
E65. The antibody of any one of E57-E64, comprising a gp130-VH sequence at least 90%, 91%, 92%, 93%, 94%, 95%, 96%, 97%, 98%, 99%, or 100% identical to SEQ ID NO: 21, and comprising a gp130-VL sequence at least 90%, 91%, 92%, 93%, 94%, 95%, 96%, 97%, 98%, 99%, or 100% identical to SEQ ID NO: 22.
E66. The antibody of any one of E57-E65, comprising a gp130-VH sequence of SEQ ID NO: 21, and comprising a gp130-VL sequence of SEQ ID NO: 22.
E67. The antibody of any one of E57-E66, comprising a gp130-VH sequence encoded by a nucleic acid sequence of SEQ ID NO: 35.
E68. The antibody of any one of E57-E67, comprising a gp130-VL sequence encoded by a nucleic acid sequence of SEQ ID NO 36.
E69 An antibody comprising a gp130-VH sequence encoded by a nucleic acid sequence of SEQ ID NO: 35 and comprising a gp130-VL sequence encoded by a nucleic acid sequence of SEQ ID NO 36.
E70. The antibody of any one of E57-E69, further comprising an Fc domain, wherein the Fc domain is an isotype of IgA (for example IgA₁ or IgA₂), IgD, IgE, IgM, or IgG (for example IgG₁, IgG₂, IgG₃, or IgG₄).
E71. The antibody of E70, comprising an Fc domain of the isotype of IgG.
E72. The antibody of either E70 or E71, comprising an Fc domain of the isotype of IgG₁.
E73. The antibody of E72, wherein the Fc domain is of a human IgG1 comprising one or more substitutions selected from L234A, L235A and G237A (by EU numbering) or L247A, L248A and G250A (by Kabat numbering) wherein the numbering is according to human IgG1 wildtype.
E74. The antibody of E73, wherein the antibody comprises the substitutions L234A, L235A, G237A (by EU numbering) or L247A, L248A, G250A (by Kabat numbering) wherein the numbering is according to human IgG1 wildtype.
E75. The antibody of any one of E57 to E74 wherein the Fc domain comprises the substitutions D221E & L368E (by EU numbering) or D234E & L381E (by Kabat numbering); wherein the numbering is according to human IgG1 wildtype.
E76. The antibody of any one of E57 to E74 wherein the Fc domain comprises the substitutions D221R & K409R (by EU numbering) or D234R & K422R (by Kabat numbering) wherein the numbering is according to human IgG1 wildtype.
E77. The antibody of any one of E57-E76, comprising a heavy chain having the amino acid sequence of SEQ ID NO: 23 or SEQ ID NO 30.
E78. The antibody of any one of E57-E77, comprising a heavy chain having the amino acid sequence of SEQ ID NO: 23.
E79. The antibody of any one of E57-E77, comprising a heavy chain having the amino acid sequence of SEQ ID NO: 30.
E80. The antibody of any one of E57-E79, comprising a light chain having the amino acid sequence of SEQ ID NO: 24.
E81. The antibody of any one of E57-E80, comprising a heavy chain having the amino acid sequence of SEQ ID NO: 23, and a light chain having the amino acid sequence of SEQ ID NO: 24.
E82. The antibody of any one of E57-E81, wherein the antibody thereof antagonizes gp130.
E83. The antibody of any one of E57-E82, wherein the antibody binds cynomolgus gp130.
E84. The antibody of E57-E83, wherein the binding KD of the antibody to cynomolgus gp130 is within 3 orders of magnitude of the binding KD of the antibody to human gp130 as measured by SPR.
E85. An isolated antibody that that competes for binding to gp130 with a second antibody comprising a VH having the amino acid sequence of SEQ ID NO: 21, and a VL having the amino acid sequence of SEQ ID NO: 22.
E86. A pharmaceutical composition comprising a therapeutically effective amount of the antibody of any one of E57-E85 and a pharmaceutically acceptable carrier.
E87. An isolated polynucleotide encoding the antibody of any one of E57 to E87.
E88. The polynucleotide of E87, wherein said polynucleotide is RNA.
E89. The polynucleotide of E88, wherein said polynucleotide comprises at least one chemical modification.
E90. The polynucleotide of E89, wherein the chemical modification wherein is selected from pseudouridine, 1-methylpseudouridine. N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine,), 5-methoxyuridine and 2′-O-methyl uridine.
E91. The polynucleotide of E87 or E88, wherein said polynucleotide does not comprise a chemical modification.
E92. An isolated polynucleotide encoding the VH of an antibody that binds gp130, wherein the polynucleotide comprises the nucleic acid sequence of SEQ ID NO: 35.
E93. An isolated polynucleotide encoding the VL of an antibody that binds gp130, wherein the polynucleotide comprises the nucleic acid sequence of SEQ ID NO: 36.
E94. An isolated polynucleotide encoding the VH and VL of an antibody that binds gp130, wherein the polynucleotide encoding the VH comprises the nucleic acid sequence of SEQ ID NO: 35 and the polynucleotide encoding the VL comprises the nucleic acid sequence of SEQ ID NO: 36.
E95. An isolated polynucleotide encoding the heavy chain, light chain, or both, of an antibody that binds gp130, and wherein said polynucleotide comprises: the nucleic acid sequence of SEQ ID NO: 37, the nucleic acid sequence of SEQ ID NO: 38, or both.
E96. An isolated polynucleotide encoding the heavy chain, light chain, or both, of an antibody that binds gp130, and wherein said nucleic acid comprises: the nucleic acid sequence of SEQ ID NO: 40, the nucleic acid sequence of SEQ ID NO: 38, or both.
E97. A vector comprising the polynucleotide of any one of E87-E96.
E98. An isolated host cell comprising the polynucleotide of any one of E87-E96, or the vector of E97.
E99. A method of producing an isolated antibody, comprising culturing the host cell of E98 under conditions that result in production of the antibody, and recovering the antibody.
E100. The antibody of any one of E57-E85, or the pharmaceutical composition of E86, for use as a medicament.
E101. The antibody of any one of E57-E85, or the pharmaceutical composition of E86, for use in the treatment of an inflammatory disease.
E102. The antibody of any one of E57-E85, or the pharmaceutical composition of E86, for use in the treatment of one or more selected from the group consisting of inflammatory bowel disease (IBD), Crohn's disease (CD), ulcerative colitis (UC), multiple sclerosis, rheumatoid arthritis, celiac disease, asthma, allergic diseases, obesity, type 2 diabetes and cancer.
E103. The antibody of any one of E57-E85, or the pharmaceutical composition of E86, for use in the treatment of Inflammatory bowel disease (IBD).
E104. The antibody of any one of E57-E85, or the pharmaceutical composition of E86, for the use according to E46, wherein the use is for the treatment of Crohn's disease (CD) or ulcerative colitis (UC).
E105. A method of treating a medical condition, comprising administering to a subject in need thereof a therapeutically effective amount of the antibody of any one of E57-E85, or the pharmaceutical composition of E86.
E106. The method of E105, wherein the condition is selected from the group consisting of inflammatory bowel disease (IBD), Crohn's disease (CD), ulcerative colitis (UC), multiple sclerosis, rheumatoid arthritis, celiac disease, asthma, allergic diseases, obesity, type 2 diabetes and cancer.
E107. The method of E105, wherein the condition is an inflammatory disease.
E108. The method of E106 or E107, wherein the condition is Inflammatory bowel disease (IBD).
E109. The method of any one of E105 to E108, or the use of E100 to E104 comprising administering said antibody, or pharmaceutical composition, subcutaneously.
E110. The method of any one of E105 to E108, or the use of E100 to E104 wherein said antibody or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.
E111. The use of the antibody of any one of E57-E85 for the manufacture of a medicament for use in the treatment of an inflammatory bowel disease (IBD), Crohn's disease (CD), ulcerative colitis (UC), multiple sclerosis, rheumatoid arthritis, celiac disease, asthma, allergic diseases, obesity, type 2 diabetes and cancer.
E112. The use of the antibody of any one of E57-E85 for the manufacture of a medicament for use in the treatment of an inflammatory disease.
E113. The use of the antibody according to E111 or E112, wherein the condition is inflammatory bowel disease (IBD).
E114. The use of the antibody according to E113, wherein the condition is Crohn's disease (CD) and ulcerative colitis (UC).
E115. An isolated antibody comprising a first antigen binding site that binds to IL27RA and a second antigen binding site that binds to gp130, wherein the first antigen binding site comprises a VH and a VL, wherein the second antigen binding site comprises a VH and a VL, and wherein one or both of:
   a. the first antigen binding site VH comprises (i) a VH CDR1 comprising the amino acid sequence of SEQ ID NO: 1 (ii) a VH CDR2 comprising the amino acid sequence of SEQ ID NO: 2; and (iii) a VH CDR3 comprising the amino acid sequence of SEQ ID NO: 3; and
   b. the first antigen binding site VL comprises (i) a VL CDR1 comprising the amino acid sequence of SEQ ID NO: 4; (ii) a VL CDR2 comprising the amino acid sequence of SEQ ID NO: 5; and (iii) a VL CDR3 comprising the amino acid sequence of SEQ ID NO: 6.
E116. An isolated antibody comprising a first antigen binding site that binds to IL27RA and a second antigen binding site that binds to gp130, wherein the first antigen binding site comprises a VH and a VL, wherein the second antigen binding site comprises a VH and a VL, and wherein one or both of:
   a. the second antigen binding site VH comprises (i) a VH CDR1 comprising the amino acid sequence of SEQ ID NO: 15; (ii) a VH CDR2 comprising the amino acid sequence of SEQ ID NO: 16; and (iii) a VH CDR3 comprising the amino acid sequence of SEQ ID NO: 17; and
   b. the second antigen binding site VL comprises (i) a VL CDR1 comprising the amino acid sequence of SEQ ID NO: 18; (ii) a VL CDR2 comprising the amino acid sequence of SEQ ID NO: 19; and (iii) a VL CDR3 comprising the amino acid sequence of SEQ ID NO: 20.
E117. An isolated antibody comprising a first antigen binding site that binds to IL27RA and a second antigen binding site that binds to gp130, wherein the first antigen binding site comprises a VH and a VL, wherein the second antigen binding site comprises a VH and a VL, and wherein:
   a. the first antigen binding site VH comprises (i) a VH CDR1 comprising the amino acid sequence of SEQ ID NO: 1; (ii) a VH CDR2 comprising the amino acid sequence of SEQ ID NO: 2; and (iii) a VH CDR3 comprising the amino acid sequence of SEQ ID NO: 3;
   b. the first antigen binding site VL comprises (i) a VL CDR1 comprising the amino acid sequence of SEQ ID NO: 4; (ii) a VL CDR2 comprising the amino acid sequence of SEQ ID NO: 5; and (iii) a VL CDR3 comprising the amino acid sequence of SEQ ID NO: 6;
   c. the second antigen binding site VH comprises (i) a VH CDR1 comprising the amino acid sequence of SEQ ID NO: 15; (ii) a VH CDR2 comprising the amino acid sequence of SEQ ID NO: 16; and (iii) a VH CDR3 comprising the amino acid sequence of SEQ ID NO: 17; and
   d. the second antigen binding site VL comprises (i) a VL CDR1 comprising the amino acid sequence of SEQ ID NO: 18; (ii) a VL CDR2 comprising the amino acid sequence of SEQ ID NO: 19; and (iii) a VL CDR3 comprising the amino acid sequence of SEQ ID NO: 20.
E118. An isolated antibody comprising a first antigen binding site that binds to IL27RA and a second antigen binding site that binds to gp130, wherein the antibody comprises a first antigen binding site VH comprising the amino acid sequence of SEQ ID NO: 7, a first antigen binding site VL comprising the amino acid sequence of SEQ ID NO: 8, a second antigen binding site VH comprising the amino acid sequence of SEQ ID NO: 21, and a second antigen binding site VL comprising the amino acid sequence of SEQ ID NO: 22.
E119. The antibody of any one of E115 to E118, wherein the antibody is a bispecific antibody.
E120. The antibody of any one of E115 to E119, wherein the antibody comprises a Fc domain comprising a first and second Fc chain.
E121. The antibody of E120, wherein the Fc domain is an isotype of IgA (for example IgA1 or IgA2), IgD, IgE, IgM, or IgG (for example IgG1, IgG2, IgG3, or IgG4).
E122. The antibody of E120 or E121, wherein the Fc domain is an IgG1 Fc domain, IgG2 Fc domain, or an IgG4 Fc domain.
E123. The antibody of any one of E120-E122, comprising a Fc domain of an isotype of IgG1
E124. The antibody of E123, wherein the first and second Fc chains comprise one or more amino acid modifications at positions 234, 235 and 237 (by EU numbering) of human IgG1.
E124. The antibody of E124, wherein the first and second Fc chains comprise one or more substitutions selected from L234A, L235A and G237A (by EU numbering) of IgG1.
E125. The antibody of E124, wherein the first and second Fc chains comprise the substitutions L234A, L235A and G237A (by EU numbering) of IgG1.
E126. The antibody of any one of E120 to E125, wherein the first Fc chain and the second Fc chain of the Fc domain each contain one or more amino acid modifications that promote the association of the first Fc chain with the second Fc chain.
E127. The antibody of E126, comprising a first and second arm wherein:
   a. the first arm comprises the first antigen binding site and the first Fc chain, wherein the first Fc chain comprises an amino acid modification at positions 221 and 409 (EU numbering) of human IgG1, and
   b. the second arm comprises the second antigen binding site and the second Fc chain, wherein the second Fc chain comprises an amino acid modification at positions 221 and 368 (by EU numbering) of human IgG1.
E128. The antibody of E126, comprising a first and second arm wherein:
   a. the first arm comprises the first antigen binding site and the first Fc chain, wherein the first Fc chain comprises an amino acid modification at positions 221 and 368 (EU numbering) of human IgG1, and
   b. the second arm comprises the second antigen binding site and the second Fc chain, wherein the second Fc chain comprises an amino acid modification at positions 221 and 409 (by EU numbering) of human IgG1.
E129. The antibody of E126, comprising a first and second arm wherein:
   a. the first arm comprises the first antigen binding site and the first Fc chain, wherein the first Fc chain comprises the substitutions D221R & K409R (by EU numbering), of human IgG1,
   b. the second arm comprises the second antigen binding site and the second Fc chain, wherein the second Fc chain comprises the substitutions D221E & L368E (by EU numbering) of human IgG1.
E130. The antibody of E126, comprising a first and second arm wherein:
   a. the first arm comprises the first antigen binding site and the first Fc chain, wherein the first Fc chain comprises the substitutions D221E & L368E (by EU numbering), of human IgG1.
   b. the second arm comprises the second antigen binding site and the second Fc chain, wherein the second Fc chain comprises the substitutions D221R & K409R (by EU numbering), of human IgG1.
E131. An antibody that binds to both IL27RA and gp130, comprising a first heavy chain and a first light chain, and a second heavy chain and second light chain, wherein the first heavy chain and the first light chain comprise a first antigen binding site that binds to IL27RA, and the second heavy chain and the second light chain comprise a second antigen binding site that binds to gp130, wherein the first antibody heavy chain comprises the amino acid sequence of SEQ ID NO:27, the first antibody light chain comprises the amino acid sequence of SEQ ID NO: 14, the second antibody heavy chain comprises the amino acid sequence of SEQ ID NO: 30, and the second antibody light chain comprises the amino acid sequence of SEQ ID NO: 34.
E132. The antibody of any one of E115 to E131, wherein the antibody has a higher binding affinity for IL27RA than for gp130 as measured by SPR.
E133. The antibody of any one of E115 to E132, wherein the antibody has a binding affinity at least 10-fold higher for IL27RA than for gp130 as measured by SPR.
E134. The antibody of any one of E115 to E133, wherein the antibody has a binding affinity at least 100-fold higher for IL27RA than for gp130 as measured by SPR.
E135. The antibody any one of E115 to E134, wherein the antibody has a binding affinity at least 1000-fold higher for IL27RA than for gp130 as measured by SPR.
E136. The antibody of any one of E115 to E135, wherein the antibody binds to human IL27RA with an affinity of less than 1 nM when measured by SPR
E137. The antibody of any one of E115 to E136, wherein the antibody binds to human gp130 with an affinity of less than 1000 nM when measured by SPR
E138. The antibody of any one of E115 to E135, wherein the antibody binds to human IL27RA with an affinity of between 0.01 nm and 5 nM between 0.05 nm and 1 nM or between 0.1 and 1 nM and binds to human gp130 with an affinity of between 10 nm and 1000 nM, between 50 nm and 1000 nM between 100 nm and 1000 nM, between 10 nm and 500 nM or between 10 nm and 250 nM when measured by SPR
E139. The antibody of any one of E115 to E135, wherein the antibody binds to human IL27RA with an affinity of between 0.1 and 5 nM and binds to human gp130 with an affinity of between 50 nm and 1000 nM when measured by SPR.
E140. The antibody of any one of E115 to E139, wherein the antibody binds to human IL27RA with an affinity of between 0.1 and 1 nM and binds to human gp130 with an affinity of between 100 nm and 500 nM when measured by SPR
E141. The antibody of any one of E115 to E140, wherein the antibody is characterized by an EC50 of less than 10 nm in a phosphorylated STAT1 CD3+ T cell fluorescent flow cytometry assay.
E142. The antibody of any one of E115 to E141, wherein the antibody is characterized by an EC50 of less than 5 nm in a flow cytometry assay of phosphorylated STAT1 CD3+ T cells.
E143. The antibody of any one of E115 to E142, wherein the antibody is characterized by an EC50 of less than 1 nm in flow cytometry assay of phosphorylated STAT1 CD3+ T cells.
E144. The antibody of any one of E115 to E143, wherein the antibody is characterized by an EC50 of less than 0.5 nm in a flow cytometry assay of phosphorylated STAT1 CD3+ T cells.
E145. The antibody of any one of E115 to E144, wherein the antibody is characterized by an EC50 of between 10 nm and 0.01 nm, between 10 nm and 0.1 nm, between 1 nm and 0.01 nm or between 1 nm and 0.1 nm a in a flow cytometry assay of phosphorylated STAT1 CD3+ T cells.
E146. The antibody of any one of E115 to E145, wherein the antibody is characterized by an EC50 of between 1 nm and 0.1 nm a in a flow cytometry assay of phosphorylated STAT1 CD3+ T cells
E147. The antibody of any one of E115 to E146, wherein the antibody is characterized by an EC50 of less than 1 nm in a flow cytometry assay of phosphorylated STAT3 in CD3+ T cells.
E148. The antibody of any one of E115 to E147, wherein the antibody is characterized by an EC50 of less than 0.5 nm in a flow cytometry assay of phosphorylated STAT3 in CD3+ T cells.
E149. The antibody of any one of E115 to E148, wherein the antibody is characterized by an EC50 of less than 10 nm, less than 1 nm, less than 0.5 nm, less than 0.3 nm, less than 0.1 nm in a flow cytometry assay of phosphorylated STAT3 in CD3+ T cells.
E150. The antibody of any one of E115 to E149, wherein the antibody is characterized by an EC50 of less than 0.3 nm in a flow cytometry assay of phosphorylated STAT3 in CD3+ T cells.
E151. The antibody of any one of E115 to E150, wherein the antibody is characterized by an EC50 of between 10 nm and 0.01 nm, between 10 nm and 0.1 nm, between 1 nm and 0.01 nm or between 1 nm and 0.1 nm in a flow cytometry assay of phosphorylated STAT3 in CD3+ T cells.
E152. The antibody of any one of E115 to E151, wherein the antibody is characterized by an EC50 of between 1 nm and 0.1 nm in a flow cytometry assay of phosphorylated STAT3 in CD3+ T cells.
E153. The antibody of any one of E115 to E152, wherein the antibody is characterized by activation of phosphorylated STAT 1 and STAT 3 in CD3+ T cells.
E154. The antibody of any one of E115-E153, wherein the antibody binds cynomolgus IL27RA and cynomolgus gp130.
E155. The antibody of any one of E115-E154, wherein the antibody is capable of down-regulating pathogenic cytokine production.
E156. The antibody of E155, wherein the antibody is capable of down-regulating 11-17 production in T helper cells.
E157. The antibody of any one of E115 to E156, wherein the antibody is characterized by an IC50 of less than 0.05 nm as measured by Il-17 immunoassay.
E158. The antibody of any one of E115 to E157, wherein the antibody is characterized by an 1050 of less than 0.01 nm as measured by Il-17 immunoassay.
E159. The antibody of any one of E115 to E158, wherein the antibody is characterized by an IC50 of between 1 nm and 0.0001 nm, between 1 nm and 0.01 nm, between 0.1 nm and 0.0001 nm, between 0.1 nm and 0.001 nm, between 0.01 nm and 0.0001 nm or between 0.01 nm and 0.001 nm as measured by Il-17 immunoassay.
E160. The antibody of any one of E115 to E159, wherein the antibody is characterized by an 1050 of between 0.01 nm and 0.001 nm as measured by Il-17 immunoassay.
E161. The antibody of any one of E115-E160, wherein the antibody is capable of promoting regulatory T cell differentiation.
E162. The antibody of E161, wherein the regulatory T cells are natural Treg (nTreg) and inducible Tregs (iTreg).
E163. The antibody of any one of E115-E162, wherein the antibody is capable of upregulating indoleamine-pyrrole 2,3-dioxygenase (IDO1) expression
E164. The antibody of any one of E115-E163, wherein the antibody is capable of upregulating indoleamine-pyrrole 2,3-dioxygenase (IDO1) expression in CD14+ human monocytes and/or human colonocytes.
E165. The antibody of any one of E115-E164, wherein the antibody is characterized by an EC50 of less than 100 nm by LC-MS assay determination of kynurenine production.
E166. The antibody of any one of E115-E165, wherein the antibody is characterized by an EC50 of less than 10 nm by LC-MS assay determination of kynurenine production.
E167. The antibody of any one of E115-E166, wherein the antibody is characterized by an EC50 of between 100 nm and 0.1 nm, between 100 nm and 1 nm, between 10 nm and 0.1 nm or between 10 nm and 1 nm by LC-MS assay determination of kynurenine production.
E168. The antibody of any one of E115-E167, wherein the antibody is characterized by an EC50 of between 10 nm and 1 nm by LC-MS assay determination of kynurenine production.
E169. A pharmaceutical composition comprising a therapeutically effective amount of the antibody of any one of E115-E168 and a pharmaceutically acceptable carrier.
E170. A vector comprising the polynucleotide of either E37 or E39.
E171. A vector comprising the polynucleotide of either E94 or E96.
E172. An isolated host cell comprising the polynucleotide of E37 or E39, or the vector of E170.
E173. An isolated host cell comprising the polynucleotide of either E94 or E96 or the vector of E171.
E174. An isolated host cell comprising:
   i) the polynucleotide of either E94 or E96 or the vector of E170 and
   ii) the polynucleotide of either E94 or E96 or the vector of E171.
E175. The antibody of any one of E115-E168, or the pharmaceutical composition of E169, for use in the treatment of an inflammatory disease.
E176. The antibody of any one of E115-E168, or the pharmaceutical composition of E169, for use in the treatment of one or more selected from the group consisting of inflammatory bowel disease (IBD), Crohn's disease (CD), ulcerative colitis (UC), multiple sclerosis, rheumatoid arthritis, celiac disease, asthma, allergic diseases, obesity, type 2 diabetes, and cancer.
E177. The antibody of any one of E115-E168, or the pharmaceutical composition of E169, for use in the treatment of Inflammatory bowel disease (IBD).
E178. The antibody of any one E115-E168, or the pharmaceutical composition of E169, for the use according to E46, wherein the use is for the treatment of Crohn's disease (CD) or ulcerative colitis (UC).
E179. A method of treating a medical condition, comprising administering to a subject in need thereof a therapeutically effective amount of the antibody of any one of E115-E168, or the pharmaceutical composition of E169.
E180. The method of E179, wherein the condition is selected from the group consisting of inflammatory bowel disease (IBD), Crohn's disease (CD), ulcerative colitis (UC), multiple sclerosis, rheumatoid arthritis, celiac disease, asthma, allergic diseases, obesity, type 2 diabetes and cancer.
E181. The method of E179, wherein the condition is an inflammatory disease.
E182. The method of E180 or E181, wherein the condition is Inflammatory bowel disease (IBD).
E183. The method of any one of E179 to E182, or the use of E175 to E178 comprising administering said antibody, or pharmaceutical composition, subcutaneously.
E184. The method or the use of any one of E175 to E183, wherein said antibody or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.
E185. The use of the antibody of any one of E115-E168 for the manufacture of a medicament for use in the treatment of an inflammatory bowel disease (IBD), Crohn's disease (CD), ulcerative colitis (UC), multiple sclerosis, rheumatoid arthritis, celiac disease, asthma, allergic diseases, obesity, type 2 diabetes, and cancer.
E186. The use of the antibody of any one of E115-E168 for the manufacture of a medicament for use in the treatment of an inflammatory disease.
E187. The use of the antibody according to E185 or E186, wherein the condition is inflammatory bowel disease (IBD).
E188. The use of the antibody according to E187, wherein the condition is Crohn's disease (CD) and ulcerative colitis (UC.
E189. An isolated antibody comprising a first antigen binding site that binds to IL27RA and a second antigen binding site that binds to gp130, wherein the first antigen binding site comprises a VH and a VL, wherein the second antigen binding site comprises a VH and a VL, and wherein the antibody has a binding affinity for human IL27RA at least two orders of magnitude lower the antibody binding affinity to human gp130.
E190. The antibody of any one of E1-E189, comprising a first antigen binding site that binds to IL27RA and a second antigen binding site that binds to gp130, wherein the first antigen binding site comprises a VH and a VL, wherein the second antigen binding site comprises a VH and a VL, and wherein the antibody has a binding affinity for human IL27RA at least two orders of magnitude lower the antibody binding affinity to human gp130.
E191. The antibody of any one of E189-E190, wherein the antibody has a binding affinity for human IL27RA at least three orders of magnitude lower the antibody binding affinity to human gp130.
E192 The antibody of E189-E191, wherein the antibody binds to human IL27RA with an affinity of less than 1 nM when measured by SPR.
E193. The antibody of E189-E192, wherein the antibody binds to human gp130 with an affinity of greater than 100 nM when measured by SPR.
Without wishing to be bound by any particular theory, binding of the antibody to the IL27R subunit IL27RA without the engagement of the gp130 subunit of the IL27R acts to antagonize the IL27 receptor.
E194. An isolated antibody that specifically binds to IL27RA, comprising a heavy chain variable region (IL27RA-VH) encoded by the plasmid deposited at the ATCC and having ATCC Accession No. PTA-127622.
E195. An isolated antibody that specifically binds to IL27RA, comprising a light chain variable region (IL27RA-VL) encoded by the plasmid deposited at the ATCC and having ATCC Accession No. PTA-127623.
E196. An isolated antibody that specifically binds to IL27RA, comprising a heavy chain variable region (IL27RA-VH) encoded by the plasmid deposited at the ATCC and having ATCC Accession No. PTA-127622, and a light chain variable region (IL27RA-VL) sequence encoded by the plasmid deposited at the ATCC and having ATCC Accession No. PTA-127623.
E197. An isolated antibody that specifically binds to IL27RA, comprising a heavy chain (IL27RA-HC) encoded by the plasmid deposited at the ATCC and having ATCC Accession No. PTA127626.
E198. An isolated antibody that specifically binds to IL27RA, comprising a light chain (IL27RA-LC) encoded by the plasmid deposited at the ATCC and having ATCC Accession No. PTA-127627.
E199. An isolated antibody that specifically binds to IL27RA, comprising a heavy chain (IL27RA-HC) encoded by the plasmid deposited at the ATCC and having ATCC Accession No. PTA-127626, and a light chain (IL27RA-LC) sequence encoded by the plasmid deposited at the ATCC and having ATCC Accession No. PTA-127627.
E200. An isolated antibody that specifically binds to gp130, comprising a heavy chain variable region (gp130-VH) encoded by the plasmid deposited at the ATCC and having ATCC Accession No. PTA-127624.
E201. An isolated antibody that specifically binds to gp130, comprising a light chain variable region (gp130-VL) encoded by the plasmid deposited at the ATCC and having ATCC Accession No. PTA-127625.
E202. An isolated antibody that specifically binds to gp130, comprising a heavy chain variable region (gp130-VH) encoded by the plasmid deposited at the ATCC and having ATCC Accession No. PTA-127624, and a light chain variable region (gp130-VL) sequence encoded by the plasmid deposited at the ATCC and having ATCC Accession No. PTA-127624.
E203. An isolated antibody that specifically binds to gp130, comprising a heavy chain (gp130-HC) encoded by the plasmid deposited at the ATCC and having ATCC Accession No. PTA-127628.
E204. An isolated antibody that specifically binds to gp130, comprising a light chain (gp130-LC) encoded by the plasmid deposited at the ATCC and having ATCC Accession No. PTA-127629.
E205. An isolated antibody that specifically binds to gp130, comprising a heavy chain (gp130-VH) encoded by the plasmid deposited at the ATCC and having ATCC Accession No. PTA-127628, and a light chain (gp130-LC) sequence encoded by the plasmid deposited at the ATCC and having ATCC Accession No. PTA-127629.
E206. An isolated antibody that specifically binds to IL27RA and gp130, comprising a heavy chain variable region (IL27RA-VH) encoded by the plasmid deposited at the ATCC and having ATCC Accession No. PTA-127622, and a light chain variable region (IL27RA-VL) sequence encoded by the plasmid deposited at the ATCC and having ATCC Accession No. PTA-127623, and further comprising a heavy chain variable region (gp130-VH) encoded by the plasmid deposited at the ATCC and having ATCC Accession No. PTA-127624, and a light chain variable region (gp130-VL) sequence encoded by the plasmid deposited at the ATCC and having ATCC Accession No. PTA-127625.
E207. An isolated antibody that specifically binds to IL27R and gp130, comprising a heavy chain (IL27RA-HC) encoded by the plasmid deposited at the ATCC and having ATCC Accession No. PTA-127626, and a light chain (IL27RA-LC) sequence encoded by the plasmid deposited at the ATCC and having ATCC Accession No. PTA-127627, and further comprising a heavy chain (gp130-VH) encoded by the plasmid deposited at the ATCC and having ATCC Accession No. PTA-127628, and a light chain (gp130-LC) sequence encoded by the plasmid deposited at the ATCC and having ATCC Accession No. PTA127629.
E208. The isolated antibody of any one of E194-E207, further comprising the antibody of any one of E1-E30, E43-E47, E57-E85, E100-E104, E115-E168, E175-E178, E189-E193
   The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and updated versions thereof.

### Definitions

Unless otherwise defined herein, scientific and technical terms used in connection with the present invention have the meanings that are commonly understood by those of ordinary skill in the art.

As used herein, the singular form “a”, “an”, and “the” include plural references unless indicated otherwise. For example, “an” antibody includes one or more antibodies.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Any example(s) following the term “e.g.” or “for example” is not meant to be exhaustive or limiting.

As used herein, the term “about” when used to modify a numerically defined parameter (e.g., the dose of ***) means that the parameter may vary by as much as 10% below or above the stated numerical value for that parameter. For example, a dose of about 5 mg means 5%±10%, i.e. it may vary between 4.5 mg and 5.5 mg.

### Antibody

An “antibody” refers to an immunoglobulin molecule capable of specific binding to a target, such as a polypeptide, carbohydrate, polynucleotide, lipid, etc., through at least one antigen binding site, located in the variable region of the immunoglobulin molecule. As used herein, the term “antibody” can encompass any type of antibody (e.g. monospecific, bispecific), and includes portions of intact antibodies that retain the ability to bind to a given antigen (e.g. an “antigen-binding fragment”), and any other modified configuration of an immunoglobulin molecule that comprises an antigen binding site.

An antibody includes an antibody of any class, such as IgG, IgA, or IgM (or sub-class thereof), and the antibody need not be of any particular class. Depending on the antibody amino acid sequence of the constant region of its heavy chains (HC), immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy chain constant regions that correspond to the different classes of immunoglobulins are called alpha, delta, epsilon, gamma, and mu, respectively. The subunit structures and three-dimensional configurations of different classes of immunoglobulins are well known.

Examples of antibody antigen-binding fragments and modified configurations include (i) a Fab fragment (a monovalent fragment consisting of the VL, VH, CL and CH1 domains); (ii) a F(ab′)2 fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al., Science 1988; 242:423-426 and Huston et al., Proc. Natl. Acad. Sci. 1988 USA 85:5879-5883. Other forms of single chain antibodies, such as diabodies are also encompassed.

In addition, further encompassed are antibodies that are missing a C-terminal lysine (K) amino acid residue on a heavy chain polypeptide (e.g. human IgG1 heavy chain comprises a terminal lysine). As is known in the art, the C-terminal lysine is sometimes clipped during antibody production, resulting in an antibody with a heavy chain lacking the C-terminal lysine. Alternatively, an antibody heavy chain may be produced using a nucleic acid that does not include a C-terminal lysine.

### Variable Region

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonincal class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, the extended definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The extended definition is the combination of the Kabat and Chothia definitions. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab lnitio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any one or more of Kabat, Chothia, extended, AbM, contact, or conformational definitions.

### Pfabat Numbering Method Developed for Consistent Antibody Numbering

The Pfabat numbering method is a defined algorithm for consistent antibody numbering, based on the Kabat numbering system (Sequences of Proteins of Immunological Interest, Fifth Edition by Kabat et al., NIH Publication NO: 91-3242, 1991). Unlike many other computational implementations of Kabat numbering, Pfabat numbers entire human IgG1 heavy and light chains, including the constant (C) regions and heavy chain hinge. Unless stated otherwise, the numbering system used herein is the Pfabat system.

### Constant Region

A “constant region” of an antibody refers to the constant region of the antibody light chain or the constant region of the antibody heavy chain, either alone or in combination. An IgG heavy chain constant region contains three sequential immunoglobulin domains (CH1, CH2, and CH3), with a hinge region between the CH1 and CH2 domains. An IgG light chain constant region contains a single immunoglobulin domain (CL).

### Fc Domain and Fc Chain

A “Fc domain” refers to the portion of an immunoglobulin (Ig) molecule that correlates to a crystallizable fragment obtained by papain digestion of an Ig molecule. As used herein, the term relates to the 2-chained constant region of an antibody, each chain excluding the first constant region immunoglobulin domain. Within an Fc domain, there are two “Fc chains” (e.g. a “first Fc chain” and a “second Fc chain”). “Fc chain” generally refers to the C-terminal portion of an antibody heavy chain. Thus, Fc chain refers to the last two constant region immunoglobulin domains (CH2 and CH3) of IgA, IgD, and IgG heavy chains, and the last three constant region immunoglobulin domains of IgE and IgM heavy chains, and optionally the flexible hinge N-terminal to these domains.

Although the boundaries of the Fc chain may vary, the human IgG heavy chain Fc chain is usually defined to comprise residues C226 or P230 to its carboxyl-terminus, wherein the numbering is according to the EU index of Edelman et al., Proc. Natl. Acad. Sci. USA 1969; 63(1):78-85 and as described in Kabat et al., 1991. Typically, the Fc chain comprises from about amino acid residue 236 to about 447 of the human IgG1 heavy chain constant region. “Fc chain” may refer to this polypeptide in isolation, or in the context of a larger molecule (e.g. in an antibody heavy chain or Fc fusion protein).

A “functional” Fc domain refers to an Fc domain that possesses at least one effector function of a native sequence Fc domain. Exemplary “effector functions” include Clq binding; complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation, etc. Such effector functions generally require the Fc domain to be combined with a binding domain (e.g., an antibody variable region) and can be assessed using various assays known in the art for evaluating such antibody effector functions.

A “native sequence” Fc chain refers to a Fc chain that comprises an amino acid sequence identical to the amino acid sequence of an Fc chain found in nature. A “variant” Fc chain comprises an amino acid sequence which differs from that of a native sequence Fc chain by virtue of at least one amino acid modification

### Monoclonal Antibody

A “monoclonal antibody” (mAb) refers to an antibody that is derived from a single copy or clone, including e.g., any eukaryotic, prokaryotic, or phage clone. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. In another example, monoclonal antibodies may be isolated from phage libraries such as those generated using the techniques described in McCafferty et al., 1990, Nature 348:552-554.

### Human Antibody

A “human antibody” refers to an antibody which possesses an amino acid sequence which corresponds to that of an antibody produced by a human or has been made using any technique for making fully human antibodies. For example, fully human antibodies may be obtained by using commercially available mice that have been engineered to express specific human immunoglobulin proteins, or by library (e.g. phage, yeast, or ribosome) display techniques for preparing fully human antibodies. This definition of a human antibody specifically excludes a humanized antibody comprising non-human antigen binding residues.

### Chimeric Antibody

A “chimeric antibody” refers to an antibody in which the variable region sequences are derived from one species and the constant region sequences are derived from another species, such as an antibody in which the variable region sequences are derived from a mouse antibody and the constant region sequences are derived from a human antibody.

### Humanized Antibody

A “humanized” antibody refers to a non-human (e.g. murine) antibody that is a chimeric antibody that contains minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

### Antigen

An “antigen” refers to the molecular entity used for immunization of an immunocompetent vertebrate to produce the antibody that recognizes the antigen or to screen an expression library (e.g., phage, yeast or ribosome display library, among others) for antibody selection. Herein, antigen is termed more broadly and is generally intended to include target molecules that are specifically recognized by the antibody, thus including fragments or mimics of the molecule used in an immunization process for raising the antibody or in library screening for selecting the antibody.

### Epitope

An “epitope” refers to the area or region of an antigen to which an antibody specifically binds, e.g., an area or region comprising residues that interact with the antibody, as determined by any method well known in the art. There are many methods known in the art for mapping and characterizing the location of epitopes on proteins, including solving the crystal structure of an antibody-antigen complex, competition assays, gene fragment expression assays, epitope mapping, and synthetic peptide-based assays, as described, for example, in Chapter 11 of Harlow and Lane, Using Antibodies, a Laboratory Manual, Cold Spring Harbor Laboratory Press, Cold Spring Harbor, New York, 1999. In addition or alternatively, during the discovery process, the generation and characterization of antibodies may elucidate information about desirable epitopes. From this information, it is then possible to competitively screen antibodies for binding to the same epitope.

In addition, the epitope to which an antibody binds can be determined in a systematic screening by using overlapping peptides derived from the antigen and determining binding by the antibody. According to the gene fragment expression assays, the open reading frame encoding the antigen can be fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis.

Certain epitopes can also be identified by using large libraries of random peptide sequences displayed on the surface of phage particles (phage libraries) or yeast (yeast display). Alternatively, a defined library of overlapping peptide fragments can be tested for binding to the test antibody in simple binding assays. In an additional example, mutagenesis of an antigen, domain swapping experiments and alanine scanning mutagenesis can be performed to identify residues required, sufficient, or necessary for epitope binding.

At its most detailed level, the epitope for the interaction between the antigen and the antibody can be defined by the spatial coordinates defining the atomic contacts present in the antigen-antibody interaction, as well as information about their relative contributions to the binding thermodynamics. At a less detailed level, the epitope can be characterized by the spatial coordinates defining the atomic contacts between the antigen and antibody. At a further less detailed level the epitope can be characterized by the amino acid residues that it comprises as defined by a specific criterion, e.g., by distance between atoms (e.g., heavy, i.e., non-hydrogen atoms) in the antibody and the antigen. At a further less detailed level the epitope can be characterized through function, e.g., by competition binding with other antibodies. The epitope can also be defined more generically as comprising amino acid residues for which substitution by another amino acid will alter the characteristics of the interaction between the antibody and antigen (e.g. using alanine scanning).

From the fact that descriptions and definitions of epitopes, dependent on the epitope mapping method used, are obtained at different levels of detail, it follows that comparison of epitopes for different antibodies on the same antigen can similarly be conducted at different levels of detail.

Epitopes described at the amino acid level, e.g., determined from an X-ray crystallography, Nuclear Magnetic Resonance (NMR) spectroscopy, hydrogen/deuterium exchange Mass Spectrometry (H/D-MS), are said to be identical if they contain the same set of amino acid residues. Epitopes are said to overlap if at least one amino acid is shared by the epitopes. Epitopes are said to be separate (unique) if no amino acid residue is shared by the epitopes.

Yet another method which can be used to characterize an antibody is to use competition assays with other antibodies known to bind to the same antigen, to determine if an antibody of interest binds to the same epitope as other antibodies. Competition assays are well known to those of skill in the art. Epitopes characterized by competition binding are said to be overlapping if the binding of the corresponding antibodies are mutually exclusive, i.e., binding of one antibody excludes simultaneous or consecutive binding of the other antibody. The epitopes are said to be separate (unique) if the antigen is able to accommodate binding of both corresponding antibodies simultaneously.

Epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds.

### Binding Affinity

The term “binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art. Low-affinity antibodies generally bind antigen slowly and tend to dissociate readily, whereas high-affinity antibodies generally bind antigen faster and tend to remain bound longer. In particular, the term “binding affinity” is intended to refer to the dissociation rate of a particular antigen-antibody interaction. The K_{D} is the ratio of the rate of dissociation, also called the “off-rate (k_{off})” or “k_{d}” to the association rate, or “on-rate (kₒₙ)” or “kₐ”. Thus, KD equals k_{off}/kₒₙ (or k_{d}/kₐ) and is expressed as a molar concentration (M). It follows that the smaller the K_{D}, the stronger the affinity of binding. Therefore, a K_{D} of 1 μM indicates weaker binding affinity compared to a K_{D} of 1 nM. K_{D} values for antibodies can be determined using methods well established in the art. One exemplary method for determining the K_{D} of an antibody is by using surface plasmon resonance (SPR), typically using a biosensor system such as BIACORE system. BIACORE kinetic analysis comprises analyzing the binding and dissociation of an antigen from chips with immobilized molecules (e.g., molecules comprising epitope binding domains), on their surface. Another method for determining the K_{D} of an antibody is by using Bio-Layer Interferometry, typically using OCTET® technology (Octet QK^{e} system, ForteBio). Alternatively, or in addition, a KinExA (Kinetic Exclusion Assay) assay, available from Sapidyne Instruments (Boise, ID) can also be used.

### Monospecific Antibody

A “monospecific antibody” refers to an antibody that comprises one or more antigen binding sites per molecule such that any and all binding sites of the antibody specifically recognize the identical epitope on the antigen. Thus, in cases where a monospecific antibody has more than one antigen binding site, the binding sites compete with each other for binding to one antigen molecule.

### Bispecific Antibody

A “bispecific antibody” refers to a molecule that has binding specificity for at least two different epitopes. In some embodiments, bispecific antibodies can bind simultaneously two different antigens. In other embodiments, the two different epitopes may reside on the same antigen.

### Half Maximal Effective Concentration (EC₅₀)

The term “half maximal effective concentration (EC₅₀)” refers to the concentration of a therapeutic agent which causes a response halfway between the baseline and maximum after a specified exposure time. The therapeutic agent may cause inhibition or stimulation. The EC₅₀ value is commonly used, and is used herein, as a measure of potency.

### Agonist

An “agonist” refers to a substance which promotes (i.e., induces, causes, enhances, or increases) the biological activity or effect of another molecule. The term agonist encompasses substances (such as an antibody) which bind to a molecule to promote the activity of that molecule.

### Antagonist

An “antagonist” refers to a substance that prevents, blocks, inhibits, neutralizes, or reduces a biological activity or effect of another molecule, such as a receptor. The term antagonist encompasses substances (such as an antibody) which bind to a molecule to prevent or reduce the activity of that molecule.

### Compete

The term “compete”, as used herein with regard to an antibody, means that a first antibody binds to an epitope in a manner sufficiently similar to the binding of a second antibody such that the result of binding of the second antibody with its cognate epitope is detectably decreased in the presence of the first antibody compared to the binding of the second antibody in the absence of the first antibody. The alternative, where the binding of the first antibody to its epitope is also detectably decreased in the presence of the second antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing or cross-competing antibodies are encompassed and can be useful for the methods disclosed herein.

### Fc Receptor

An “Fc receptor” (FcR) refers to a receptor that binds to the Fc region of an antibody. In some embodiments, an FcR is a native human FcR. In some embodiments, an FcR is one which binds an IgG antibody (a gamma receptor) and includes receptors of the FcgRI, FcgRII, and FcgRII) subclasses, including allelic variants and alternatively spliced forms of those receptors. FcgRII receptors include FcgRIIA (an “activating receptor”) and FcgRIIB (an “inhibiting receptor”), which have similar amino acid sequences that differ primarily in the cytoplasmic domains thereof. Activating receptor FcgRIIA contains an immunoreceptor tyrosine-based activation motif (ITAM) in its cytoplasmic domain. Inhibiting receptor FcgRIIB contains an immunoreceptor tyrosine-based inhibition motif (ITIM) in its cytoplasmic domain, (see, e.g., Daeron, Annu. Rev. Immunol. 1997; 15:203-234). FcRs are reviewed, for example, in Ravetch and Kinet, Annu. Rev. Immunol 1991; 9:457-92; Capel et al., Immunomethods 1994; 4:25-34; and de Haas et al., J. Lab. Clin. Med. 1995; 126:330-41. Other FcRs, including those to be identified in the future, are encompassed by the term “Fc receptor” herein. The term “Fc receptor” also includes the neonatal receptor, FcRn, which is responsible for the transfer of maternal IgGs to the fetus (Guyer et al., J. Immunol. 1976; 117:587 and Kim et al., J. Immunol. 1994; 24:249) and regulation of homeostasis of immunoglobulins. Methods of measuring binding to FcRn are known (see, e.g., Ghetie and Ward., Immunol. Today 1997; 18(12):592-598; Ghetie et al., Nature Biotechnology, 1997; 15(7):637-640; Hinton et al., J. Biol. Chem. 2004; 279(8):6213-6216; WO 2004/92219).

### Effector Cells

An “effector cell” refers to a leukocyte which express one or more FcRs and performs effector functions. In certain embodiments, effector cells express at least FcgRII) and perform ADCC effector function(s). Examples of leukocytes which mediate ADCC include peripheral blood mononuclear cells (PBMC), natural killer (NK) cells, monocytes, macrophages, cytotoxic T cells, and neutrophils. Effector cells may be isolated from a native source, e.g., from blood.

### Antibody-Dependent Cell-Mediated Cytotoxicity (ADCC)

The term “antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enable these cytotoxic effector cells to bind specifically to an antigen-bearing target cell and subsequently kill the target cell with cytotoxins. The primary cells for mediating ADCC, NK cells, express FcgRIII only, whereas monocytes express FcgRI, FcgRII, and FcgRIII. To assess ADCC activity of a molecule of interest, an in vitro ADCC assay, such as that described in U.S. Pat. Nos. 5,500,362, 5,821,337 or 6,737,056, may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. (USA) 1998; 95:652-656. Additional antibodies with altered Fc region amino acid sequences and increased or decreased ADCC activity are described, e.g., in U.S. Pat. Nos. 7,923,538, and 7,994,290.

### Enhanced ADCC Activity

The term “enhanced ADCC activity” refers to an antibody that is more effective at mediating ADCC in vitro or in vivo compared to the parent antibody, wherein the antibody and the parent antibody differ in at least one structural aspect, and when the amounts of such antibody and parent antibody used in the assay are essentially the same. In some embodiments, the antibody and the parent antibody have the same amino acid sequence, but the antibody is afucosylated while the parent antibody is fucosylated. In some embodiments, ADCC activity will be determined using an in vitro ADCC assay, but other assays or methods for determining ADCC activity, e.g. in an animal model etc., are contemplated. In some embodiments, an antibody with enhanced ADCC activity has enhanced affinity for FcgRIIIA.

### Altered FcR Binding or ADCC Activity

The term “altered” FcR binding affinity or ADCC activity refers to an antibody which has either enhanced or diminished activity for one or more of FcR binding activity or ADCC activity compared to a parent antibody, wherein the antibody and the parent antibody differ in at least one structural aspect. An antibody that “displays increased binding” to an FcR binds at least one FcR with better affinity than the parent antibody. An antibody that “displays decreased binding” to an FcR, binds at least one FcR with lower affinity than a parent antibody. Such antibodies that display decreased binding to an FcR may possess little or no appreciable binding to an FcR, e.g., 0-20 percent binding to the FcR compared to a native sequence IgG Fc region.

### Complement Dependent Cytotoxicity (CDC)

The term “Complement dependent cytotoxicity” or “CDC” refers to the lysis of a target cell in the presence of complement. Activation of the classical complement pathway is initiated by the binding of the first component of the complement system (Clq) to antibodies (of the appropriate subclass), which are bound to their cognate antigen. To assess complement activation, a CDC assay, e.g., as described in Gazzano-Santoro et al., J. Immunol. Methods 1996; 202: 163, may be performed. Antibodies with altered Fc region amino acid sequences and increased or decreased Clq binding capability are described, e.g., in U.S. Pat. Nos. 6,194,551, 7,923,538, 7,994,290 and WO 1999/51642.

### Host Cell

A “host cell” refers to an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

### Vector

A “vector” refers to a construct, which is capable of delivering, and, preferably, expressing, one or more gene(s) or sequence(s) of interest (e.g. an antibody-encoding gene) in a host cell. Examples of vectors include, but are not limited to plasmids and viral vectors, and may include naked nucleic acids, or may include nucleic acids associated with delivery-aiding materials (e.g. cationic condensing agents, liposomes, etc). Vectors may include DNA or RNA. An “expression vector” as used herein refers to a vector that includes at least one polypeptide-encoding gene, at least one regulatory element (e.g. promoter sequence, poly(A) sequence) relating to the transcription or translation of the gene. Typically, a vector used herein contains at least one antibody-encoding gene, as well as one or more of regulatory elements or selectable markers. Vector components may include, for example, one or more of the following: a signal sequence; an origin of replication; one or more marker genes; suitable transcriptional controlling elements (such as promoters, enhancers and terminator). For translation, one or more translational controlling elements may also be included such as ribosome binding sites, translation initiation sites, and stop codons.

### Isolated

An “isolated” molecule (e.g. antibody) refers to a molecule that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art.

### Polypeptide/Protein

A “polypeptide” or “protein” (used interchangeably herein) refers to a chain of amino acids of any length. The chain may be linear or branched. The chain may comprise one or more of modified amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

### Polynucleotide/Nucleic Acid

A “polynucleotide” or “nucleic acid,” (used interchangeably herein) refers to a chain of nucleotides of any length, and includes DNA and RNA. The nucleotides can be deoxyribonucleotides, ribonucleotides, modified nucleotides or bases or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside.

### Conservative Substitution

A “conservative substitution” refers to replacement of one amino acid by a biologically, chemically or structurally similar residue. Biologically similar means that the substitution does not destroy a biological activity. Structurally similar means that the amino acids have side chains with similar length, such as alanine, glycine and serine or a similar size. Chemical similarity means that the residues have the same charge or are both hydrophilic or hydrophobic. Particular examples include the substitution of a hydrophobic residue, such as isoleucine, valine, leucine or methionine with another, or the substitution of one polar residue for another, such as the substitution of arginine for lysine, glutamic acid for aspartic acid or glutamine for asparagine, serine for threonine, and the like. Particular examples of conservative substitutions include the substitution of a hydrophobic residue such as isoleucine, valine, leucine or methionine for one another, the substitution of a polar residue for another, such as the substitution of arginine for lysine, glutamic acid for aspartic acid, or glutamine for asparagine, and the like. Conservative amino acid substitutions typically include, for example, substitutions within the following groups: glycine, alanine, valine, isoleucine, leucine; aspartic acid, glutamic acid; asparagine, glutamine; serine, threonine; lysine, arginine; and phenylalanine, tyrosine.

### Identity

The term “identity” or “identical to” refers to the overall relatedness between polymeric molecules, e.g., between nucleic acid molecules (e.g., DNA molecules or RNA molecules) or between polypeptide molecules. “Identity” measures the percent of identical matches between two or more sequences with gap alignments addressed by a particular mathematical model of computer programs (e.g. algorithms), which are well known in the art. Calculation of percentage identity between two polymeric molecules may be calculated from such an alignment as N/T*′100, where N is the number of positions at which the sequences share an identical residue and T is the total number of positions compared including gaps and either including or excluding overhanging sequences. In one embodiment, overhanging sequences are included in the calculation.

The terms “increase,” improve,” “decrease” or “reduce” refer to values that are relative to a baseline measurement, such as a measurement in the same individual prior to initiation of treatment described herein, or a measurement in a control individual or subject (or multiple control individuals or subjects) in the absence of the treatment described herein. In some embodiments, a “control individual” is an individual afflicted with the same form of disease or injury as an individual being treated. In some embodiments, a “control individual” is an individual that is not afflicted with the same form of disease or injury as an individual being treated.

### Excipient

The term ‘excipient’ refers to any material which, which combined with an active ingredient of interest (e.g. antibody), allow the active ingredient to retain biological activity. The choice of excipient will to a large extent depend on factors such as the mode of administration, the effect of the excipient on solubility and stability, and the nature of the dosage form. As used herein, “excipient”” includes any and all solvents, dispersion media, coatings, antibacterial and antifungal agents, isotonic and absorption delaying agents, carriers, diluents and the like that are physiologically compatible. Examples of an excipient include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition.

### Treating

The terms “treating”, “treat” or “treatment” refer to any type of treatment, e.g. such as to relieve, alleviate, or slow the progression of the patient's disease, disorder or condition or any tissue damage associated with the disease. In some embodiments, the disease, disorder or condition is inflammatory bowel disease (IBD). In some embodiments, the disease, disorder or condition is Crohn's disease (CD). In some embodiments, the disease, disorder or condition is ulcerative colitis (UC).

### Prevent

The terms “prevent” or “prevention” refer to one or more of delay of onset, reduction in frequency, or reduction in severity of at least one sign or symptom of a particular disease, disorder or condition (e.g., inflammatory bowel disease (IBD)) In some embodiments, prevention is assessed on a population basis such that an agent is considered to “prevent” a particular disease, disorder or condition if a statistically significant decrease in the development, frequency or intensity of one or more symptoms of the disease, disorder or condition is observed in a population susceptible to the disease, disorder or condition. Prevention may be considered complete when onset of disease, disorder or condition has been delayed for a predefined period of time.

### Subject

The terms “subject, “individual” or “patient,” (used interchangeably herein), refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development. In some embodiments, a subject is a patient with inflammatory bowel disease (IBD).

### Therapeutically Effective Amount

The term “therapeutically effective amount” refers to the amount of active ingredient that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting or slowing further development of the pathology or symptomatology); and
(3) ameliorating the disease; for example, ameliorating a disease, condition or disorder in an individual that is experiencing or displaying the pathology or symptomatology of the disease, condition or disorder (i.e., reversing the pathology or symptomatology).

### Antibodies to IL27RA

The disclosure provides antibodies that bind to the interleukin 27 receptor subunit alpha (IL27RA) also known as cytokine receptor-Like 1, cytokine receptor WSX-1, Zcytor1, T-cell and cytokine receptor type 1.

As used herein, the term IL27RA includes variants, isoforms, homologs, orthologs and paralogs of IL27RA. In some embodiments, an antibody disclosed herein cross-reacts with IL27RA from species other than human, such as IL27RA of cynomolgus monkey, as well as different forms of IL27RA. In some embodiments, an antibody may be completely specific for human IL27RA and may not exhibit species cross-reactivity (e.g., does not bind mouse IL27RA) or other types of cross-reactivity. As used herein the term IL27RA refers to naturally occurring human IL27RA unless contextually dictated otherwise. Therefore, an “IL27RA antibody” “anti-IL27RA antibody” or other similar designation means any antibody (as defined herein) that binds or reacts with IL27RA, an isoform, fragment or derivative thereof. The full length, mature form of IL27RA, as represented by UniProtKB/Swiss-Prot accession number Q6UWB1 is herein provided as SEQ ID NO: 41 The full length, mature form of mouse IL27RA, as represented by UniProtKB/Swiss-Prot accession number 070394 is herein provided as SEQ ID NO: 44 The full length, mature form of cynomolgus IL27RA, as represented by UniProtKB/Swiss-Prot accession number A0A2K5WKA4 is herein provided as SEQ ID NO:42.

Without wishing to be bound by any particular theory, binding of the antibody to the IL27R subunit IL27RA without the engagement of the gp130 subunit of the IL27R acts to antagonize the IL27 receptor.

“Biological function” or “biological activity” of IL27RA is meant to modifying inflammation and modifying regulatory functions in innate immune and T cells. The biological function or biological activity of IL27RA can, but need not be, mediated by the interaction between IL27 and its ligands.

In some embodiments, an anti-IL27RA antibody of the disclosure encompasses an antibody that one or both of i) competes for binding to human IL27RA with or ii) binds the same epitope as, an antibody having the amino acid sequence of a heavy chain variable region set forth as SEQ ID NO:31 and the amino acid sequence of a light chain variable region set forth as SEQ ID NO:32.

Anti-IL27RA antibodies of the present disclosure can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, an anti-IL27RA antibody is a monoclonal antibody. In some embodiments, an anti-IL27RA antibody is a human or humanized antibody. In some embodiments, an anti-IL27RA antibody is a chimeric antibody.

In some embodiments, the invention provides an antibody having a light chain variable region (VL) sequence and a heavy chain variable region (VH) sequence as found in Table 13, or variants thereof.

The invention also provides CDR portions of antibodies to IL27RA. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, or contact CDRs. In other words, in embodiments with more than one CDR, the CDRs may be any of Kabat, Chothia, extended, AbM, conformational, contact CDRs or combinations thereof.

In some embodiments, the antibody comprises three CDRs of any one of the heavy chain variable regions shown in Table 13. In some embodiments, the antibody comprises three CDRs of any one of the light chain variable regions shown in Table 13. In some embodiments, the antibody comprises three CDRs of any one of the heavy chain variable regions shown in Table 13, and three CDRs of any one of the light chain variable regions shown in Table 13.

In some embodiments, the antibody comprises three light chain CDRs and three heavy chain CDRs from Table 13

In some embodiments, the antibody comprises one or both of i) the full-length heavy chain, with or without the C-terminal lysine, or ii) the full-length light chain of anti-IL27RA antibody of anti-1127RA-4701 or anti-1127RA 4880 EE. The amino acid sequences of the full-length heavy chain and light chain for antibodies are shown below in Table 13.

Table 13 also provides for heavy chain and light chain sequences for mAbs of the invention.

In some embodiments, the antibody may comprise a heavy chain variable region (IL27RA-VH) and a light chain variable region (IL27RA-VL), comprising the CDR-H1, CDR-H2, and CDR-H3 sequences of SEQ ID NO: 7, and the CDR-L1, CDR-L2, and CDR-L3 sequences of SEQ ID NO: 8.

In some embodiments, the antibody may comprise a heavy chain variable region (IL27RA-VH) and a light chain variable region (IL27RA-VL), comprising a CDR-H1 sequence according to SEQ ID NO: 1; a CDR-H2 sequence according to SEQ ID NO: 2; a CDR-H3 sequence according to SEQ ID NO: 3 and comprising a CDR-L1 sequence according to SEQ ID NO: 4; a CDR-L2 sequence according to SEQ ID NO: 5, and a CDR-L3 sequence according to SEQ ID NO: 6.

In some embodiments, the antibody comprises an IL27RA-VH sequence encoded by a nucleic acid sequence of SEQ ID NO: 31 and comprising a IL27RA-VL sequence encoded by a nucleic acid sequence of SEQ ID NO 32.

In some embodiments, the antibody comprises an IL27RA-VH framework sequence that is derived from a human germline VH sequence selected from the group consisting of DP7, DP10, DP35, DP47, DP50, DP51, DP54, and DP77. In some embodiments the IL27RA-VH framework sequence may be derived from a human germline DP54 sequence.

In some embodiments, the antibody comprises a IL27RA-VL framework sequence derived from a human germline VL sequence selected from the group consisting of DPK1, DPK3, DPK4, DPK5, DPK7, DPK8, and DPK9. In some embodiments, the antibody comprises a IL27RA-VL framework sequence may be derived from a human germline DPK9, sequence.

In some embodiments, the IL27RA-VL framework sequence and a IL27RA-VH framework sequence may comprise one or more amino acid substitutions, additions, or deletions, while still retaining functional and structural similarity with the germline from which it was derived. In some embodiments, the one or both the IL27RA-VL framework sequence and the IL27RA-VH framework sequence may be at least 90%, 91%, 92%, 93%, 94%, 95%, 96%, 97%, 98%, 99%, or 100% identical to the human germline sequence from which it was derived. In some embodiments one or both of the IL27RA-VL framework sequence or the IL27RA-VH framework sequence may be identical to the human germline sequence from which it was derived.

In some embodiments the IL27RA-VH sequence may be at least 90%, 91%, 92%, 93%, 94%, 95%, 96%, 97%, 98%, 99%, or 100% identical to SEQ ID NO: 7, and comprising a IL27RA-VL sequence at least 90%, 91%, 92%, 93%, 94%, 95%, 96%, 97%, 98%, 99%, or 100% identical to SEQ ID NO: 8.

In some embodiments the IL27RA antibody comprises a heavy chain having the amino acid sequence of SEQ ID NO: 13 or SEQ ID NO 27. In some embodiments the L27RA antibody comprises a light chain having the amino acid sequence of SEQ ID NO: 14.

In some embodiments the IL27RA antibody comprises a heavy chain having the amino acid sequence of SEQ ID NO: 13, and a light chain having the amino acid sequence of SEQ ID NO: 14. In some embodiments the IL27RA antibody comprises a heavy chain having the amino acid sequence of SEQ ID NO: 27, and a light chain having the amino acid sequence of SEQ ID: 14.

Antibodies to gp130

The disclosure provides antibodies that bind to glycoprotein 130 (gp130) also known as interleukin 6 receptor subunit beta, CD130 and Interleukin-6 cytokine family signal transducer.

As used herein, the term gp130 includes variants, isoforms, homologs, orthologs and paralogs of gp130. In some embodiments, an antibody disclosed herein cross-reacts with gp130 from species other than human, such as gp130 of cynomolgus monkey, as well as different forms of gp130. In some embodiments, an antibody may be completely specific for human gp130 and may not exhibit species cross-reactivity (e.g., does not bind mouse gp130) or other types of cross-reactivity. As used herein the term gp130 refers to naturally occurring human gp130 unless contextually dictated otherwise. Therefore, an “gp130 antibody” “anti-gp130 antibody” or other similar designation means any antibody (as defined herein) that binds or reacts with gp130, an isoform, fragment or derivative thereof. The full length, mature form of gp130, as represented by UniProtKB/Swiss-Prot accession number P40189 is herein provided as SEQ ID NO: 45. The full length, mature form of mouse gp130, as represented by UniProtKB/Swiss-Prot accession number Q00560 is herein provided as SEQ ID NO: 48. The full length, mature form of cynomolgus gp130, as represented by Gene ID number 3572 is herein provided as SEQ ID NO:46.

The biological function or biological activity of IL27RA can, but need not be, mediated by the interaction between gp130 and its ligands.

In some embodiments, an anti-gp130 antibody of the disclosure encompasses an antibody that one or both of i) competes for binding to human gp130 with or ii) binds the same epitope as, an antibody having the amino acid sequence of a heavy chain variable region set forth as SEQ ID NO:20 and the amino acid sequence of a light chain variable region set forth as SEQ ID NO: 21.

Anti gp130 antibodies of the present disclosure can encompass monoclonal antibodies, polyclonal antibodies, antibody fragments (e.g., Fab, Fab′, F(ab)₂, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, an anti-gp130 antibody is a monoclonal antibody. In some embodiments, an anti-gp130 antibody is a human or humanized antibody. In some embodiments, an anti-gp130 antibody is a chimeric antibody.

In some embodiments, the invention provides an antibody having a light chain variable region (VL) sequence and a heavy chain variable region (VH) sequence as found in Table 13, or variants thereof.

The invention also provides CDR portions of antibodies to gp130. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, or contact CDRs. In other words, in embodiments with more than one CDR, the CDRs may be any of Kabat, Chothia, extended, AbM, conformational, contact CDRs or combinations thereof.

In some embodiments, the antibody comprises three CDRs of any one of the heavy chain variable regions shown in Table 13 In some embodiments, the antibody comprises three CDRs of any one of the light chain variable regions shown in Table 13. In some embodiments, the antibody comprises three CDRs of any one of the heavy chain variable regions shown in Table 13, and three CDRs of any one of the light chain variable regions shown in Table 13

Table 13 provides examples of CDR sequences of anti-gp130 antibodies provided herein. In some embodiments, the antibody comprises three light chain CDRs and three heavy chain CDRs from Table 13.

In some embodiments, the antibody comprises one or both of i) the full-length heavy chain, with or without the C-terminal lysine, or ii) the full-length light chain of anti-gp130 antibody anti-gp130 4574 or anti-gp130-4875 RR The amino acid sequences of the full-length heavy chain and light chain for antibodies anti-gp130 4574 or anti-gp130-4875 RR are shown below in 13.

In some embodiments, the antibody that specifically binds to glycoprotein 130 (gp130), comprises a heavy chain variable region (gp130-VH) and a light chain variable region (gp130-VL), comprising the CDR-H1, CDR-H2, and CDR-H3 sequences of SEQ ID NO: 21, and the CDR-L1, CDR-L2, and CDR-L3 sequences of SEQ ID NO: 22.

In some embodiments, the antibody comprises a heavy chain variable region (gp130-VH) and a light chain variable region (gp130-VL), comprising a CDR-H1 sequence according to SEQ ID NO: 15; a CDR-H2 sequence according to SEQ ID NO: 16; a CDR-H3 sequence according to SEQ ID NO: 17 and comprising a CDR-L1 sequence according to SEQ ID NO: 18; a CDR-L2 sequence according to SEQ ID NO: 19, and a CDR-L3 sequence according to SEQ ID NO: 20.

In some embodiments, the antibody comprises a gp130-VH framework sequence derived from a human germline VH sequence selected from the group consisting of DP7, DP10, DP35, DP47, DP50, DP51, DP54, and DP77. In some embodiment, the antibody comprises a gp130-VH framework sequence derived from a human germline DP10 sequence.

In some embodiments, the antibody comprises a IL27RA-VL framework sequence derived from a human germline VL sequence selected from the group consisting of DPK1, DPK3, DPK4, DPK5, DPK7, DPK8, and DPK9. In some embodiments, the antibody comprises a gp130-VL framework sequence derived from a human germline DPK9 sequence.

In some embodiments, the gp130-VL framework sequence and a gp130-VH framework sequence may comprise one or more amino acid substitutions, additions, or deletions, while still retaining functional and structural similarity with the germline from which it was derived. In some embodiments, the one or both the gp130-VL framework sequence and the IL27RA-VH framework sequence may be at least 90%, 91%, 92%, 93%, 94%, 95%, 96%, 97%, 98%, 99%, or 100% identical to the human germline sequence from which it was derived. In some embodiments one or both of the gp130-VL framework sequence or the gp130-VH framework sequence may be identical to the human germline sequence from which it was derived.

In some embodiments, the antibody comprises a gp130-VH sequence at least 90%, 91%, 92%, 93%, 94%, 95%, 96%, 97%, 98%, 99%, or 100% identical to SEQ ID NO: 21, and comprising a gp130-VL sequence at least 90%, 91%, 92%, 93%, 94%, 95%, 96%, 97%, 98%, 99%, or 100% identical to SEQ ID NO: 22.

In some embodiments, the antibody comprises a gp130-VH sequence of SEQ ID NO: 21 and comprising a gp130-VL sequence of SEQ ID NO: 22.

In some embodiments, the antibody comprises a gp130-VH sequence encoded by a nucleic acid sequence of SEQ ID NO: 35. In some embodiments, the antibody comprises a gp130-VL sequence encoded by a nucleic acid sequence of SEQ ID NO 36. In some embodiments, the antibody comprises a gp130-VH sequence encoded by a nucleic acid sequence of SEQ ID NO: 35 and comprising a gp130-VL sequence encoded by a nucleic acid sequence of SEQ ID NO 36.

In some embodiments, the antibody comprises a heavy chain having the amino acid sequence of SEQ ID NO: 23 or SEQ ID NO 30. 45. In some embodiments, the antibody comprises a light chain having the amino acid sequence of SEQ ID NO: 24. In some embodiments, the antibody comprises a heavy chain having the amino acid sequence of SEQ ID NO: 23, and a light chain having the amino acid sequence of SEQ ID NO: 24. In some embodiments, the antibody comprises a heavy chain having the amino acid sequence of SEQ ID NO: 30, and a light chain having the amino acid sequence of SEQ ID NO: 24.

Antibodies to IL27RA and gp130

IL-27 has been implicated as playing a key role in autoimmune, inflammatory diseases and agonism of the receptor provides a means by which the pathogenic immune response associated with autoimmune, inflammatory diseases may be down-regulated. Accordingly, and without wishing to be bound by any particular theory, the bispecific IL27RA/gp130 antibody disclosed herein, for example the antibody defined as mAb-4894 in the examples, acts as an agonist for IL27R and binds to the IL27 receptor subunits IL27RA and gp130 induce an immune modulating effect. Examples of the immune modulating effect of the bispecific IL27RA/gp130 antibody includes dampening intestinal inflammation and promoting gut barrier integrity.

IL-27 acts through a heterodimeric receptor consisting of IL27RA and gp130 chains, mediating signaling through signal transducer and activator of transcription (STAT)1 and STAT3. Thus, the bispecific antibody disclosed herein binding to both IL27RA and gp130 is capable of binding to both subunits of the IL27RA/gp130 heterodimer and induce the phosphorylation of STAT 1 and 3 signaling, as shown in Example 8, in a similar manner to the native IL27R ligand, IL27.

Without wishing to be bound by any particular theory, the immunosuppressive effect of the IL27RA/gp130 antibody may be due to multiple actions of the antibody resulting from activation of the IL27 receptor, which include the following.

The IL27RA/gp130 antibody may decrease Th17 and Th2 responses such that the antibody down-regulates GATA-3 and IL-13 expression during Th2 cell differentiation and down-regulates IL-17A expression during Th17 cell differentiation, as shown in Example 9. CD4+ T-helper cells play a variety of important roles in the development and maintenance of various autoimmune diseases including IBD. Based upon the differentially secreted cytokine panels which in turn mediate distinctive cellular activities, CD4+ T helper cells can be characterized into distinct subtypes: Th1, Th2, and Th17 cells.

The IL27RA/gp130 antibody may upregulate IDO1 expression in the CD14+ monocyte cytoplasm and in human colonocytes thus providing immunoprotective and immunosuppressive effect, as shown in Example 8. IDO1 is a cytosolic enzyme with a heme (Fe2+) prosthetic group that catalyzes tryptophan (Trp) catabolism and converts it to kynurenine (Kyn). The IDO1 pathway was originally described as an innate immune mechanism that defended the host organism against infections. Elevated levels of 001 strongly inhibit the proliferation and induces apoptosis of effector T cells and the accumulating Trp metabolites induces the differentiation of Tregs collectively giving rise to immunosuppression. The immunoprotective and immunosuppressive roles of 001 and Trp metabolites are tightly controlled by the stoichiometry of available local factors. The resultant effect of these local activities modulates 001 expression and helps maintain global immune homeostasis and peripheral immune tolerance.

The IL27RA/gp130 antibody may induce PD-L1 expression as shown in Example 8. PD-L1 (CD274) is the dominant inhibitory ligand of PD-1 (Programmed cell death protein 1, also called CD279). Engagement of PD-1 by PD-L1 alters the activity of T cells in many ways, such as inhibiting T cell proliferation, survival, cytokine production, and other effector functions.

The IL27RA/gp130 antibody may upregulate IL-10 gene expression and LAG3 expression, which deliver negative immune modulatory signals on engagement with T cell activation. Regulatory T (Treg) cells are essential for maintaining peripheral tolerance, preventing autoimmune diseases, and limiting chronic inflammatory diseases. There are two types of Tregs: natural Tregs (nTreg) and inducible Tregs (iTreg). The IL27RA/gp130 antibody induces more CD4+CD25+FOXP3+ iTreg from naïve CD4+ T cells compared to control antibody, upregulated the LAG3+ population, upregulated the Tim-3 expression level, and increased the Tim-3+ cell population as well.

The IL27RA/gp130 antibody may induce an inhibitory effect on the allogenic T cell proliferation mediated by three types of DCs immature DCs, immunogenic DCs and tolerogenic DCs, whereby the IL27RA/gp130 antibody significantly down-regulated cell surface CD83 expression and up-regulated ILT4 expression.

In one embodiment, the IL27RA/gp130 antibody does induces an anti-inflammatory response. In one embodiment, the IL27RA/gp130 antibody does not induce a pro-inflammatory response. In one embodiment, the gp130 antibody does not induce IFN gamma expression from Th1 cells.

The gp130 subunit is broadly distributed and present on other gp130-containing receptors such as interleukin 6 receptor (IL-6R), interleukin 11 receptor (IL-11RO, Oncostatin M Receptor (OSMR), and LIF Receptor Subunit Alpha (LIFR) leaving the potential for off target effects associated with binding to non-target gp130 subunit containing receptors.

Thus, in one embodiment, and as shown in Example 6, the binding affinities of the two arms of the IL27RA/gp130 antibody have been tuned such that the antibody has higher affinity to the IL27RA subunit, when compared to the more broadly distributed gp130 subunit. This differential affinity enables sufficient potency for agonist activity in IL27R while minimizing binding to other gp130-containing receptors. In some embodiments, the IL27RA/gp130 antibody has a binding affinity at least 10-fold higher for IL27RA than for gp130 as measured by SPR. In some embodiments, the IL27RA/gp130 antibody has a binding affinity at least 100-fold higher for IL27RA than for gp130 as measured by SPR. In some embodiments, the IL27RA/gp130 antibody has a binding affinity at least 1000-fold higher for IL27RA than for gp130 as measured by SPR.E136. In some embodiments, the IL27RA/gp130 antibody binds to human IL27RA with an affinity of less than 1 nM when measured by SPR. In some embodiments, the IL27RA/gp130 antibody binds to human gp130 with an affinity of less than 1000 nM when measured by SPR In some embodiments, the IL27RA/gp130 antibody binds to human IL27RA with an affinity of between 0.01 nm and 5 nM between 0.05 nm and 1 nM or between 0.1 and 1 nM and binds to human gp130 with an affinity of between 10 nm and 1000 nM, between 50 nm and 1000 nM between 100 nm and 1000 nM, between 10 nm and 500 nM or between 10 nm and 250 nM when measured by SPR. In some embodiments, the IL27RA/gp130 antibody binds to human IL27RA with an affinity of between 0.1 and 5 nM and binds to human gp130 with an affinity of between 50 nm and 1000 nM when measured by SPR. In some embodiments, the IL27RA/gp130 antibody binds to human IL27RA with an affinity of between 0.1 and 1 nM and binds to human gp130 with an affinity of between 100 nm and 500 nM when measured by SPR.

The disclosure provides antibodies that bind to IL27RA and gp130. As used herein, the terms IL27RA and gp130 include variants, isoforms, homologs, orthologs and paralogs of IL27RA and gp130 respectively. In some embodiments, an antibody disclosed herein cross-reacts with one or more of IL27RA and gp130 from species other than human, such as IL27RA and gp130 of cynomolgus monkey. In some embodiments, an antibody may be completely specific for IL27RA and gp130 and may not exhibit species cross-reactivity or other types of cross-reactivity. As used herein the term TL1A and gp130 refers to naturally occurring human IL27RA and gp130 unless contextually dictated otherwise. A “IL27RA/gp130 antibody” “anti-IL27RA/gp130 antibody” or other similar designation means any antibody (as defined herein) that binds or reacts with IL27RA and gp130, an isoform, fragment or derivative thereof

In some embodiments, the invention provides a IL27RA/gp130 antibody having a light chain variable region (VL) sequence and a heavy chain variable region (VH) sequence as found in Table 13 or 14 or variants thereof.

The invention also provides CDR portions of IL27RA/gp130 antibodies. Determination of CDR regions is defined. In some embodiments, the IL27RA/gp130 antibody comprises the three CDRs of the IL27RA antibody of Table 13 and three CDRs of the gp130 antibody of Table 13 or 14.

In some embodiments, the disclosure provides anti-IL27RA/gp130 antibodies containing variations of the CDRs, VH, VL, HC, and LC regions shown in Table 13 and 14, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in one or more of Table 13 and 14. These amounts are not meant to be limiting and increments between the recited percentages are specifically envisioned as part of the disclosure.

In certain embodiments, antibodies described herein comprise an Fc domain. The Fc domain can be derived from IgA (e.g., IgA₁ or IgA₂), IgG, IgE, or IgG (e.g., IgG₁, IgG₂, IgG₃, or IgG₄). In some embodiments, an anti-IL27RA antibody is an IgG₂ antibody. In some embodiments, an anti-IL27RA antibody is an IgG₁ antibody.

The invention encompasses modifications to the variable regions, CDRs and heavy chain and light chain sequences shown in Table 13 or 14. For example, the invention includes antibodies comprising functionally equivalent variable regions and CDRs which do not significantly affect their properties as well as variants which have enhanced or decreased activity or affinity. For example, the amino acid sequence may be mutated to obtain an antibody with the desired binding affinity to IL27RA and gp130. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs

A modification or mutation may also be made in a framework region or constant region to increase the half-life of an antibody provided herein. See, e.g., PCT Publication No. WO 00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. In some embodiments, no more than one to five conservative amino acid substitutions are made within the framework region or constant region. In other embodiments, no more than one to three conservative amino acid substitutions are made within the framework region or constant region. According to the invention, a single antibody may have mutations in any one or more of the CDRs or framework regions of the variable domain or in the constant region.

In some embodiments, the antibody comprises a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), or does not activate microglia; or has reduced activities (compared to the unmodified antibody) in any one or more of the following: triggering complement mediated lysis, stimulating ADCC, or activating microglia. Different modifications of the constant region may be used to achieve optimal level or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 1999, 29:2613-2624; PCT Publication No. WO99/058572.

In some embodiments, the antibody can comprise amino acid modifications at one or more positions L234, L235 and G237 (by EU numbering) or L247, L248 and G250 (by Kabat numbering) in human isotype IgG₁.

In some embodiments, the antibody can comprise amino acid modifications at positions L234, L235 and G237 (by EU numbering) or L247, L248 and G250 (by Kabat numbering) in human IgG₁.

In some embodiments, the antibody can comprise the amino acid modifications of one or more of L234A, L235A and G237A (by EU numbering) or L247A, L248A and G250A (by Kabat numbering) in human IgG₁.

In some embodiments, the antibody can comprise the amino acid modifications of one or more of L234A, L235A and G237A (by EU numbering) or L247A, L248A and G250A (by Kabat numbering) in human IgG2.

In some embodiments, the antibody can comprise the amino acid modifications of one or more of L234A, L235A and G237A (by EU numbering) or L247A, L248A and G250A (by Kabat numbering) in human IgG₃.

In some embodiments, the antibody can comprise the amino acid modifications of one or more of L234A, L235A and G237A (by EU numbering) or L247A, L248A and G250A (by Kabat numbering) in human IgG₄.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

In some embodiments, the disclosure provides anti-antibodies containing variations of the variable regions, CDRs or heavy chain and light chain sequences shown in Table 13, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in Table 13. These amounts are not meant to be limiting and increments between the recited percentages are specifically envisioned as part of the disclosure.

The invention also encompasses fusion proteins comprising one or more components of the antibodies disclosed herein. In some embodiments, a fusion protein may be made that comprises all or a portion of an antibody of the invention linked to another polypeptide. In another embodiment, only the variable domains of the antibody are linked to the polypeptide. In another embodiment, the VH domain of an antibody is linked to a first polypeptide, while the VL domain of an antibody is linked to a second polypeptide that associates with the first polypeptide in a manner such that the VH and VL domains can interact with one another to form an antigen binding site. In another embodiment, the VH domain is separated from the VL domain by a linker such that the VH and VL domains can interact with one another. The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

In addition to binding to an epitope on IL27RA and gp130, the anti-IL27RA/gp130 antibody of the disclosure can mediate a biological activity, as is shown in the examples, for the bispecific anti-IL27RA/gp130 antibody mAb-4894.

That is, the disclosure includes an isolated antibody that specifically binds IL27RA and gp130 and mediates at least one detectable activity selected from the following:
i) down-regulating pathogenic cytokine production. In some embodiments, the anti-IL27RA/gp130 antibody down-regulating pathogenic cytokine production by reducing interleukin 17 production in T helper cells, for example type 17 (Th17), which can be measured by immunoassay and is described in Example 9. Thus, in some embodiments, the anti-IL27RA/gp130 antibody has an IC50 of less than 0.05 nm as measured by 11-17 immunoassay. In some embodiments, the anti-IL27RA/gp130 antibody has an IC50 of less than 0.01 nm as measured by 11-17 immunoassay. In some embodiments, the anti-IL27RA/gp130 antibody has an IC50 of between 1 nm and 0.0001 nm, between 1 nm and 0.01 nm, between 0.1 nm and 0.0001 nm, between 0.1 nm and 0.001 nm, between 0.01 nm and 0.0001 nm or between 0.01 nm and 0.001 nm as measured by 11-17 immunoassay. In some embodiments, the anti-IL27RA/gp130 antibody has an IC50 of between 0.01 nm and 0.001 nm as measured by 11-17 immunoassay. In some embodiments, the anti-IL27RA/gp130 antibody may down-regulating pathogenic cytokine production by inhibiting T helper cell type 2 (Th2) responses, for example reducing interleukin 13 (IL-13) production and GATA-3 expression;
ii) promoting regulatory T cell differentiation, for example promoting differentiation of natural Treg (nTreg) and inducible Tregs (iTreg) as shown in Example 8. In some embodiments, the iTreg is characterized by expression of CD4+CD25+FOXP3+.
iii) upregulate expression of immune checkpoint molecules such as Tim-3, LAG-3 and IL-10. In some embodiments, expression of IL-10, Tim-3 and LAG-3 can be determined at both transcription level and protein level, for example by flow cytometry as demonstrated in Example 9.
iv) suppress T cell proliferation;
v) induce programmed death-ligand 1 (PD-L1) expression in monocytes, which can be measured by flow cytometry and is described in Example 8; and
vii) upregulate indoleamine-pyrrole 2,3-dioxygenase enzyme (IDO1) expression in colonocytes and/or monocytes, which can be measured by determining the production of kynurenine (Kyn) by liquid chromatography—mass spectrometry (LC-MS) assay to reflect IDO1 activity, as described in Example 8. Thus, in some embodiments, the anti-IL27RA/gp130 antibody has an EC50 of less than 100 nm by LC-MS assay determination of kynurenine production. In some embodiments, the anti-IL27RA/gp130 antibody has EC50 of less than 10 nm by LC-MS assay determination of kynurenine production. In some embodiments, the anti-IL27RA/gp130 antibody has an EC50 of between 100 nm and 0.1 nm, between 100 nm and 1 nm, between 10 nm and 0.1 nm or between 10 nm and 1 nm by LC-MS assay determination of kynurenine production. In some embodiments, the anti-IL27RA/gp130 antibody has an EC50 of between 10 nm and 1 nm by LC-MS assay determination of kynurenine production

Accordingly, the bispecific antibody disclosed herein, as the antibody defined as mAb-4894 in the examples, has the potential to down-regulate pathogenic T helper 17 cells (Th17) while upregulating cell surface markers associated with regulatory T cells (Treg) and more specifically the two subpopulations of natural Treg (nTreg) and inducible Tregs (iTreg). In addition, targeting of both IL27RA and gp130 enables the reduction of type 2 cytokines and upregulates negative regulators on monocytes and dendritic cells. The bispecific IL27R agonist has direct effects on human primary colonocytes as evident by the upregulation of indoleamine-pyrrole 2,3-dioxygenase enzyme (IDO1) which is known to be associated with immune suppression and mucosal healing.

In some embodiments the bispecific antibody comprises a first antigen binding site that binds to IL27RA and a second antigen binding site that binds to gp130, wherein the first antigen binding site comprises a VH and a VL, wherein the second antigen binding site comprises a VH and a VL, and wherein one or both of:
a. the first antigen binding site VH comprises (i) a VH CDR1 comprising the amino acid sequence of SEQ ID NO: 1 (ii) a VH CDR2 comprising the amino acid sequence of SEQ ID NO: 2; and (iii) a VH CDR3 comprising the amino acid sequence of SEQ ID NO: 3; and
b. the first antigen binding site VL comprises (i) a VL CDR1 comprising the amino acid sequence of SEQ ID NO: 4; (ii) a VL CDR2 comprising the amino acid sequence of SEQ ID NO: 5; and (iii) a VL CDR3 comprising the amino acid sequence of SEQ ID NO: 6.

In some embodiments the bispecific antibody comprises a first antigen binding site that binds to IL27RA and a second antigen binding site that binds to gp130, wherein the first antigen binding site comprises a VH and a VL, wherein the second antigen binding site comprises a VH and a VL, and wherein one or both of:
a. the second antigen binding site VH comprises (i) a VH CDR1 comprising the amino acid sequence of SEQ ID NO: 15; (ii) a VH CDR2 comprising the amino acid sequence of SEQ ID NO: 16; and (iii) a VH CDR3 comprising the amino acid sequence of SEQ ID NO: 17; and
b. the second antigen binding site VL comprises (i) a VL CDR1 comprising the amino acid sequence of SEQ ID NO: 18; (ii) a VL CDR2 comprising the amino acid sequence of SEQ ID NO: 19; and (iii) a VL CDR3 comprising the amino acid sequence of SEQ ID NO: 20.

In some embodiments the bispecific antibody comprises a first antigen binding site that binds to IL27RA and a second antigen binding site that binds to gp130, wherein the first antigen binding site comprises a VH and a VL, wherein the second antigen binding site comprises a VH and a VL, and wherein:
a. the first antigen binding site VH comprises (i) a VH CDR1 comprising the amino acid sequence of SEQ ID NO: 1; (ii) a VH CDR2 comprising the amino acid sequence of SEQ ID NO: 2; and (iii) a VH CDR3 comprising the amino acid sequence of SEQ ID NO: 3;
b. the first antigen binding site VL comprises (i) a VL CDR1 comprising the amino acid sequence of SEQ ID NO: 4; (ii) a VL CDR2 comprising the amino acid sequence of SEQ ID NO: 5; and (iii) a VL CDR3 comprising the amino acid sequence of SEQ ID NO: 6;
c. the second antigen binding site VH comprises (i) a VH CDR1 comprising the amino acid sequence of SEQ ID NO: 15; (ii) a VH CDR2 comprising the amino acid sequence of SEQ ID NO: 16; and (iii) a VH CDR3 comprising the amino acid sequence of SEQ ID NO: 17; and
d. the second antigen binding site VL comprises (i) a VL CDR1 comprising the amino acid sequence of SEQ ID NO: 18; (ii) a VL CDR2 comprising the amino acid sequence of SEQ ID NO: 19; and (iii) a VL CDR3 comprising the amino acid sequence of SEQ ID NO: 20.

In some embodiments the bispecific antibody comprises a first antigen binding site that binds to IL27RA and a second antigen binding site that binds to gp130, wherein the antibody comprises a first antigen binding site VH comprising the amino acid sequence of SEQ ID NO: 7, a first antigen binding site VL comprising the amino acid sequence of SEQ ID NO: 8, a second antigen binding site VH comprising the amino acid sequence of SEQ ID NO: 21, and a second antigen binding site VL comprising the amino acid sequence of SEQ ID NO: 22.

In some embodiments the bispecific antibody comprises a first heavy chain and a first light chain, and a second heavy chain and second light chain, wherein the first heavy chain and the first light chain comprise a first antigen binding site that binds to IL27RA, and the second heavy chain and the second light chain comprise a second antigen binding site that binds to gp130, wherein the first antibody heavy chain comprises the amino acid sequence of SEQ ID NO:27, the first antibody light chain comprises the amino acid sequence of SEQ ID NO: 14, the second antibody heavy chain comprises the amino acid sequence of SEQ ID NO: 30, and the second antibody light chain comprises the amino acid sequence of SEQ ID NO: 34.

### Polynucleotides Encoding Antibodies of the Invention

The disclosure also provides polynucleotides encoding any of the antibodies of the invention, including antibody portions and modified antibodies described herein. The invention also provides a method of making any of the antibodies and polynucleotides described herein. Polynucleotides can be made and the proteins expressed by procedures known in the art.

If desired, an antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

In some embodiments, provided herein is a polynucleotide comprising a sequence encoding one or both of the heavy chain or the light chain variable regions of an antibody provided herein. The sequence encoding the antibody of interest may be maintained in a vector in a host cell and the host cell can then be expanded and frozen for future use. Vectors (including expression vectors) and host cells are further described herein.

In some embodiments, the disclosure provides a polynucleotide encoding the amino acid sequences of any of the antibodies listed in Tables 13 or 14.

In one embodiment, the invention provides a polynucleotide encoding the amino acid sequence of the anti-IL27RA antibody

In some embodiments, the disclosure provides a polynucleotide encoding one or more anti-IL27RA antibody heavy chain polypeptides comprising an amino acid sequence selected from: SEQ ID NO: 13 or 27. In some embodiments, the disclosure provides polynucleotides encoding one or more anti-IL27RA antibody light chain polypeptide comprising an amino acid sequence of: SEQ ID NO: 14.

In some embodiments, the disclosure provides a polynucleotide encoding one or more anti-IL27RA antibody VH polypeptides comprising an amino acid sequence of: SEQ ID NO: 7. In some embodiments, the disclosure provides a polynucleotide encoding one or more anti-IL27RA antibody VL polypeptides comprising an amino acid sequence of: SEQ ID NOs: 8.

In some embodiments, the disclosure provides a polynucleotide encoding one or more anti-gp130 antibody heavy chain polypeptide comprising an amino acid sequence selected from the group consisting of: SEQ ID NOs: 23 or 30. In some embodiments, the disclosure provides a polynucleotide encoding one or more anti-gp130 antibody light chain polypeptide comprising an amino acid sequence of: SEQ ID NO: 24. In some embodiments, the disclosure provides a polynucleotide encoding one or more anti-gp130 antibody VH polypeptides comprising an amino acid sequence of: SEQ ID NO: 20 In some embodiments, the disclosure provides a polynucleotide encoding one or more anti-gp130 antibody VL polypeptides comprising an amino acid sequence of: SEQ ID NO: 21.

In some embodiments, the polynucleotide encoding the anti-IL27RA antibody HC comprises the nucleic acid sequence of SEQ ID NO: 33. In some embodiments, the polynucleotide encoding the anti-IL27RA antibody LC polypeptide comprises the nucleic acid sequence of SEQ ID NO: 34. In some embodiments, the disclosure provides polynucleotides encoding anti-IL27RA antibody HC polypeptide comprising the nucleic acid sequence of SEQ ID NO: 33 and an anti-IL27RA antibody LC polypeptide comprising the nucleic acid sequence of SEQ ID NO: 34.

In some embodiments, the polynucleotide encoding the anti-IL27RA antibody HC comprises the nucleic acid sequence of SEQ ID NO: 39. In some embodiments, the polynucleotide encoding the anti-IL27RA antibody LC polypeptide comprises the nucleic acid sequence of SEQ ID NO: 34. In some embodiments, the disclosure provides polynucleotides encoding anti-IL27RA antibody HC polypeptide comprising the nucleic acid sequence of SEQ ID NO: 39 and an anti-IL27RA antibody LC polypeptide comprising the nucleic acid sequence of SEQ ID NO: 34.

In some embodiments, the polynucleotide encoding the anti-gp130 antibody HC comprises the nucleic acid sequence of SEQ ID NO: 37. In some embodiments, the polynucleotide encoding the anti-gp130 antibody LC polypeptide comprises the nucleic acid sequence of SEQ ID NO: 38. In some embodiments, the disclosure provides polynucleotides encoding anti-gp130 antibody HC polypeptide comprising the nucleic acid sequence of SEQ ID NO: 37 and an anti-gp130 antibody LC polypeptide comprising the nucleic acid sequence of SEQ ID NO: 38.

In some embodiments, the polynucleotide encoding the anti-gp130 antibody HC comprises the nucleic acid sequence of SEQ ID NO: 40. In some embodiments, the polynucleotide encoding the anti-gp130 antibody LC polypeptide comprises the nucleic acid sequence of SEQ ID NO: 38. In some embodiments, the disclosure provides polynucleotides encoding anti-gp130 antibody HC polypeptide comprising the nucleic acid sequence of SEQ ID NO: 40 and an anti-gp130 antibody LC polypeptide comprising the nucleic acid sequence of SEQ ID NO: 38.

In some embodiments, the disclosure provides polynucleotides encoding the heavy chain, light chain, or both, of an antibody that binds gp130, and wherein said polynucleotide comprise the nucleic acid sequence of SEQ ID NO: 37, the nucleic acid sequence of SEQ ID NO: 38, or both. In some embodiments, the disclosure provides polynucleotides encoding the heavy chain, light chain, or both, of an antibody that binds gp130, and wherein said polynucleotide comprises the nucleic acid sequence of SEQ ID NO: 40, the nucleic acid sequence of SEQ ID NO: 38, or both.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions or substitutions of nucleotides. The resulting mRNA and protein may, but need not, have an altered structure or function. Alleles may be identified using standard techniques (such as hybridization, amplification or database sequence comparison).

In one embodiment, the VH and VL domains or full-length HC or LC, are encoded by separate polynucleotides. Alternatively, both VH and VL, or HC and LC, are encoded by a single polynucleotide.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules or support materials.

### Methods of Manufacture

Various techniques for the production of antibodies have been described which include the traditional hybridoma method for making monoclonal antibodies, recombinant techniques for making antibodies (including chimeric antibodies, e.g., humanized antibodies), antibody production in transgenic animals and the recently described phage display technology for preparing “fully human” antibodies.

Provided herein are methods of making any of the antibodies provided herein. The antibodies of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

Any suitable method for preparing multispecific antibodies may be used to prepare multispecific antibodies provided herein (e.g. depending on the choice of antibody features and components).

According to one approach to making multispecific antibodies, antibody variable domains with the desired binding specificities are fused to immunoglobulin constant region sequences. The fusion preferably is with an immunoglobulin heavy chain constant region, comprising at least part of the hinge, CH2 and CH3 regions. In some embodiments, the first heavy chain constant region (CH1), containing the site for light chain binding can be present in at least one of the fusions. In some embodiments, polynucleotides encoding the immunoglobulin heavy chain fusions and, if desired, the immunoglobulin light chain, may be inserted into separate expression vectors, and may be cotransfected into a suitable host organism. In other embodiments the coding sequences for two or all three polypeptide chains may be inserted into one expression vector when the expression of at least two polypeptide chains in equal ratios results in high yields or when the ratios are of no particular significance.

In one approach, the multispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the multispecific molecule, facilitates the separation of the desired multispecific compound from unwanted immunoglobulin chain combinations. This approach is described in PCT Publication No. WO 94/04690.

In another approach, the multispecific antibodies are composed of amino acid modification in the first hinge region in one arm, and the substituted amino acid in the first hinge region has an opposite charge to the corresponding amino acid in the second hinge region in another arm. This approach is described in International Patent Application No. PCT/US2011/036419 (WO2011/143545).

In another approach, the formation of a desired heteromultimeric or heterodimeric protein (e.g., bispecific antibody) is enhanced by altering or engineering an interface between a first and a second Fc chain. In this approach, the multispecific antibodies may be composed of a CH3 region, wherein the CH3 region comprises a first CH3 polypeptide and a second CH3 polypeptide which interact together to form a CH3 interface, wherein one or more amino acids within the CH3 interface destabilize homodimer formation and are not electrostatically unfavorable to homodimer formation. This approach is described in International Patent Application No. PCT/US2011/036419 (WO2011/143545). In some embodiments, one constant region of a bispecific antibody can comprise amino acid modifications at position 221 (e.g., (D221E or D221R)) in the hinge region and at position 409 (e.g., K409R (EU numbering scheme)) in the CH3 region of human IgG1, and the other constant region of the bispecific antibody can comprise amino acid modifications at positions 221 (e.g., (D221E or D221R)) in the hinge region and at position 368 (e.g., L368E (EU numbering scheme)) in the CH3 region of human IgG1. In some embodiments, one constant region of a bispecific antibody can comprise amino acid modifications at position 221 (e.g., (D221E or D221R)) in the hinge region and at position 409 (e.g., K409R (EU numbering scheme)) in the CH3 region of human IgG2, and the other constant region of the bispecific antibody can comprise amino acid modifications at positions 221 (e.g., (D221E or D221R)) in the hinge region and at position 368 (e.g., L368E (EU numbering scheme)) in the CH3 region of human IgG2. In some embodiments, one constant region of a bispecific antibody can comprise amino acid modifications at position 221 (e.g., (D221E or D221R)) in the hinge region and at position 409 (e.g., K409R (EU numbering scheme)) in the CH3 region of human IgG4, and the other constant region of the bispecific antibody can comprise amino acid modifications at positions 221 (e.g., (D221E or D221R)) in the hinge region and at position 368 (e.g., L368E (EU numbering scheme)) in the CH3 region of human IgG4.

In some embodiments, a multispecific antibody may have knob-in-hole mutations in the Fc chains. For example, in some embodiments, in a bispecific antibody having knob-in-hole mutations, the first Fc chain of the antibody Fc domain has one or more mutations to form a “knob”, and the second Fc chain of the antibody Fc domain has one or more mutations to form a “hole” (or vice-versa). Exemplary knob-in-hole engineering of antibodies is described in U.S. Pat. No. 5,731,168, PCT Publication No. WO2009089004, U.S. Publication No. 20090182127, Marvin and Zhu, Acta Pharmacologica Sincia (2005) 26(6):649-658 and Kontermann (2005) Acta Pharacol. Sin., 26:1-9.

A “knob” refers to at least one amino acid side chain which projects from the interface of a first polypeptide (e.g. first Fc chain) and is therefore positionable in a compensatory hole in an adjacent second polypeptide (e.g. second Fc chain) so as to stabilize a heterodimer, and thereby favor heterodimer formation over homodimer formation. The knob may exist in the original interface or may be introduced synthetically (e.g., by altering a nucleic acid encoding the interface). Normally, nucleic acid encoding the interface of the first polypeptide is altered to encode the knob. To achieve this, the nucleic acid encoding at least one original amino acid residue in the first polypeptide is replaced with nucleic acid encoding at least one “import” amino acid residue which has a larger side chain volume than the original amino acid residue. Certain import residues for the formation of a knob are generally naturally occurring amino acid residues and are preferably selected from arginine (R), phenylalanine (F), tyrosine (Y) and tryptophan (W).

A “hole” refers to at least one amino acid side chain which is recessed from the interface of a second polypeptide (e.g. second constant domain) and therefore accommodates a corresponding knob in an adjacent first polypeptide (e.g. first constant domain). The hole may exist in the original interface or may be introduced synthetically (e.g., by altering a nucleic acid encoding the interface). Normally, nucleic acid encoding the interface of the second polypeptide is altered to encode the hole. To achieve this, the nucleic acid encoding at least one original amino acid residue of the second polypeptide is replaced with DNA encoding at least one “import” amino acid residue which has a smaller side chain volume than the original amino acid residue. Certain import residues for the formation of a hole are usually naturally occurring amino acid residues and are preferably selected from alanine (A), serine (S), threonine (T) and valine (V).

The term “interface,” as used herein typically refers to any amino acid residue present in the domain that can be involved in first polypeptide and second polypeptide contacts. An “original amino acid” residue is one which is replaced by an “import amino acid” residue which can have a smaller or larger side chain volume than the original residue. The import amino acid residue can be a naturally occurring or non-naturally occurring amino acid residue, but preferably is the former. “Naturally occurring” amino acid residues are those residues encoded by the genetic code. By “non-naturally occurring” amino acid residue is meant a residue which is not encoded by the genetic code, but which is able to covalently bind adjacent amino acid residue(s) in the polypeptide chain. Examples of non-naturally occurring amino acid residues are norleucine, ornithine, norvaline, homoserine and other amino acid residue analogues such as those described in Ellman et al., Meth. Enzym. 202:301-336 (1991).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome.

Suitable cloning vectors may be constructed according to standard techniques, or may be selected from a large number of cloning vectors available in the art. While the cloning vector selected may vary according to the host cell intended to be used, useful cloning vectors will generally have one or more features such as i) the ability to self-replicate, ii) a single target for a particular restriction endonuclease, or iii) may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

Additionally, any number of commercially and non-commercially available cell lines that express polypeptides or proteins may be utilized in accordance with the present invention. One skilled in the art will appreciate that different cell lines might have different nutrition requirements or might require different culture conditions for optimal growth and polypeptide or protein expression, and will be able to modify conditions as needed.

### Pharmaceutical Compositions

In another embodiment, the invention comprises pharmaceutical compositions.

A “pharmaceutical composition” refers to a mixture of an antibody the invention and one or excipient.

Pharmaceutical compositions of this invention may be in a variety of forms. These include, for example, liquid, semi-solid and solid dosage forms, such as liquid solutions (e.g., injectable and infusible solutions), dispersions or suspensions, and lyophilized powders. The form depends on the intended mode of administration and therapeutic application.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); or non-ionic surfactants such as TWEEN™, PLURONICS™ or polyethylene glycol (PEG).

### Therapeutic, Diagnostic, and Other Methods

The antibodies and the antibody conjugates of the present invention are useful in various applications including, but are not limited to, therapeutic treatment methods and diagnostic treatment methods.

In some embodiments, antibodies of the invention may agonize or modulate the activity of the IL27 receptor and may be useful in the treatment, prevention, suppression and amelioration of inflammatory diseases such as IBD or diseases, disorders and conditions mediated by IL27. In another embodiment antibodies of the invention may agonize or modulate the activity of the IL27 receptor and may be useful in the treatment, prevention, suppression and amelioration of multiple sclerosis, rheumatoid arthritis, celiac disease, asthma, allergic diseases, obesity, type 2 diabetes or cancer.

In one aspect, the invention provides a method for treating inflammatory bowel disease (IBD), Crohn's disease (CD), ulcerative colitis (UC), multiple sclerosis, rheumatoid arthritis, celiac disease, asthma, allergic diseases, obesity, type 2 diabetes or cancer In one aspect, the invention provides a method for treating inflammatory bowel disease (IBD). In some embodiments, the method of treating inflammatory bowel disease (IBD), Crohn's disease (CD), ulcerative colitis (UC), multiple sclerosis, rheumatoid arthritis, celiac disease, asthma, allergic diseases, obesity, type 2 diabetes or cancer in a subject comprises administering to the subject in need thereof an effective amount of a pharmaceutical composition comprising any of the antibodies as described herein. In some embodiments, provided is a method of treating IBD in a subject, comprising administering to the subject in need thereof an effective amount of a composition comprising an antibody provided herein.

In another aspect, the invention further provides an antibody or pharmaceutical composition as described herein for use in the described method of treating inflammatory bowel disease (IBD), Crohn's disease (CD), ulcerative colitis (UC), multiple sclerosis, rheumatoid arthritis, celiac disease, asthma, allergic diseases, obesity, type 2 diabetes or cancer. In another aspect, the invention further provides an antibody or pharmaceutical composition as described herein for use in the described method of treating an autoimmune disease. In another aspect, the invention further provides an antibody or pharmaceutical composition as described herein for use in the described method of treating inflammatory bowel disease (IBD). In another aspect, the invention further provides an antibody or pharmaceutical composition as described herein for use in the described method of treating ulcerative colitis or Crohn's disease. In another aspect, the invention further provides an antibody or pharmaceutical composition as described herein for use in the described method of treating ulcerative colitis. The invention also provides the use of an antibody as described herein in the manufacture of a medicament for treating inflammatory bowel disease (IBD), Crohn's disease (CD), ulcerative colitis (UC), multiple sclerosis, rheumatoid arthritis, celiac disease, asthma, allergic diseases, obesity, type 2 diabetes or cancer.

In another aspect, provided is a method of one or more of detecting, diagnosing, or monitoring inflammatory bowel disease (IBD), Crohn's disease (CD), ulcerative colitis (UC), multiple sclerosis, rheumatoid arthritis, celiac disease, asthma, allergic diseases, obesity, type 2 diabetes or cancer. For example, the antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent.

With respect to all methods described herein, reference to antibodies also includes pharmaceutical compositions comprising the antibodies and one or more additional agents.

### Administration and Dosing

Typically, an antibody of the invention is administered in an amount effective to treat a condition as described herein. The antibodies the invention can be administered as an antibody per se, or alternatively, as a pharmaceutical composition containing the antibody.

The antibodies of the invention are administered by any suitable route in the form of a pharmaceutical composition adapted to such a route, and in a dose effective for the treatment intended.

In some embodiments, the antibodies may be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. In an embodiment, antibodies may be administered subcutaneously. The Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the antibodies of the invention or compositions containing said antibodies is based on a variety of factors, including the type, age, weight, sex and medical condition of the subject; the severity of the condition; the route of administration; and the activity of the particular antibody employed. Thus, the dosage regimen may vary widely. In one embodiment, the total daily dose of an antibody of the invention is typically from about 0.01 to about 100 mg/kg (i.e., mg antibody of the invention per kg body weight) for the treatment of the indicated conditions discussed herein. In another embodiment, total daily dose of the antibody of the invention is from about 0.1 to about 50 mg/kg, and in another embodiment, from about 0.5 to about 30 mg/kg.

The antibodies of the invention can be used alone, or in combination with one or more other therapeutic agents. The invention provides any of the uses, methods or compositions as defined herein wherein an antibody of the invention is used in combination with one or more other therapeutic agent discussed herein.

The administration of two or more agents “in combination” means that all of the agents are administered closely enough in time to affect treatment of the subject. The two or more agents may be administered simultaneously or sequentially. Additionally, simultaneous administration may be carried out by mixing the agents prior to administration or by administering the agents at the same point in time but as separate dosage forms at the same or different site of administration.

Various formulations of the antibodies of the present invention (e.g., one or more of anti-IL27RA, gp130 and anti-IL27A/gp130 antibodies) may be used for administration. In some embodiments, the antibodies may be administered neat. In some embodiments, the antibody and a pharmaceutically acceptable excipient may be in various formulations. Pharmaceutically acceptable excipients are known in the art and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 21st Ed. Mack Publishing, 2005.

In some embodiments, these agents are formulated for administration by injection (e.g., intraperitoneally, intravenously, subcutaneously, intramuscularly, etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

The antibodies (e.g., one or more of anti-IL27RA, gp130 and anti-IL27A/gp130 antibodies) as described herein can be administered using any suitable method, including by injection (e.g., intraperitoneally, intravenously, subcutaneously, intramuscularly, etc.). The antibody, e.g., monoclonal antibody or multispecific antibody, also be administered via inhalation, as described herein. Generally, for administration of the antibody of the present, the dosage depends upon the host treated and the particular mode of administration. In one embodiment, the dose range of the antibody of the present invention will be about 0.001 μg/kg body weight to about 20,000 μg/kg body weight. The term “body weight” is applicable when a patient is being treated. When isolated cells are being treated, “body weight” as used herein refers to a “total cell body weight”. The term “total body weight” may be used to apply to both isolated cell and patient treatment. All concentrations and treatment levels are expressed as “body weight” or simply “kg” in this application are also considered to cover the analogous “total cell body weight” and “total body weight” concentrations. However, those of ordinary skill in the art will recognize the utility of a variety of dosage range, for example, 0.01 μg/kg body weight to 20,000 μg/kg body weight, 0.02 μg/kg body weight to 15,000 μg/kg body weight, 0.03 μg/kg body weight to 10,000 μg/kg body weight, 0.04 μg/kg body weight to 5,000 μg/kg body weight, 0.05 μg/kg body weight to 2,500 μg/kg body weight, 0.06 μg/kg body weight to 1,000 μg/kg body weight, 0.07 μg/kg body weight to 500 μg/kg body weight, 0.08 μg/kg body weight to 400 μg/kg body weight, 0.09 μg/kg body weight to 200 μg/kg body weight or 0.1 μg/kg body weight to 100 μg/kg body weight. Further, those of skill will recognize that a variety of different dosage levels will be of use, for example, one or more selected from the group consisting of 0.0001 μg/kg, 0.0002 μg/kg, 0.0003 μg/kg, 0.0004 μg/kg, 0.005 μg/kg, 0.0007 μg/kg, 0.001 μg/kg, 0.1 μg/kg, 1.0 μg/kg, 1.5 μg/kg, 2.0 μg/kg, 5.0 μg/kg, 10.0 μg/kg, 15.0 μg/kg, 30.0 μg/kg, 50 μg/kg, 75 μg/kg, 80 μg/kg, 90 μg/kg, 100 μg/kg, 120 μg/kg, 140 μg/kg, 150 μg/kg, 160 μg/kg, 180 μg/kg, 200 μg/kg, 225 μg/kg, 250 μg/kg, 275 μg/kg, 300 μg/kg, 325 μg/kg, 350 μg/kg, 375 μg/kg, 400 μg/kg, 450 μg/kg, 500 μg/kg, 550 μg/kg, 600 μg/kg, 700 μg/kg, 750 μg/kg, 800 μg/kg, 900 μg/kg, 1 μg/kg, 5 μg/kg, 10 μg/kg, 12 μg/kg, 15 mg/kg, 20 mg/kg, and 30 mg/kg. All of these dosages are exemplary, and any dosage in-between these points is also expected to be of use in the invention. Any of the above dosage ranges or dosage levels may be employed for an antibody of the present invention. For repeated administrations over several days or longer, depending on the condition, the treatment is sustained until a desired suppression of symptoms occurs or until sufficient therapeutic levels are achieved.

Generally, for administration of antibodies provided herein, the candidate dosage can be administered daily, every week, every other week, every three weeks, every four weeks, every five weeks, every six weeks, every seven weeks, every eight weeks, every ten weeks, every twelve weeks, or more than every twelve weeks.

In some embodiments, the candidate dosage is administered daily with the dosage ranging from about any of 1 μg/kg, to 30 μg/kg, to 300 μg/kg, to 3 mg/kg, to 30 mg/kg, to 100 mg/kg or more, depending on the factors mentioned above. For example, daily dosage of about 0.01 mg/kg, about 0.03 mg/kg, about 0.1 mg/kg, about 0.3 mg/kg, about 1 mg/kg, about 2.5 mg/kg, about 3 mg/kg, about 5 mg/kg, about 10 mg/kg, about 15 mg/kg, and about 25 mg/kg may be used.

In some embodiments, the candidate dosage is administered every week with the dosage ranging from about any of 1 μg/kg, to 30 μg/kg, to 300 μg/kg, to 3 mg/kg, to 30 mg/kg, to 100 mg/kg or more, depending on the factors mentioned above. For example, a weekly dosage of about 0.01 mg/kg, about 0.03 mg/kg, about 0.1 mg/kg, about 0.3 mg/kg, about 0.5 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some embodiments, the candidate dosage is administered every two weeks with the dosage ranging from about any of 1 μg/kg, to 30 μg/kg, to 300 μg/kg, to 3 mg/kg, to 30 mg/kg, to 100 mg/kg or more, depending on the factors mentioned above. For example, a bi-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some embodiments, the candidate dosage is administered every three weeks with the dosage ranging from about any of 1 μg/kg, to 30 μg/kg, to 300 μg/kg, to 3 mg/kg, to 30 mg/kg, to 100 mg/kg or more, depending on the factors mentioned above. For example, a tri-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/k may be used.

In some embodiments, the candidate dosage is administered every month or every four weeks with the dosage ranging from about any of 1 μg/kg, to 30 μg/kg, to 300 μg/kg, to 3 mg/kg, to 30 mg/kg, to 100 mg/kg or more, depending on the factors mentioned above. For example, a monthly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/kg may be used.

In other embodiments, the candidate dosage is administered daily with the dosage ranging from about 0.01 mg to about 1200 mg or more, depending on the factors mentioned above. For example, daily dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, or about 1200 mg may be used. In one embodiment, a daily dosage of between 0.01 mg and 100 mg may be used. In one embodiment, a daily dosage of between 0.01 mg and 1 mg may be used. In one embodiment, a daily dosage of between 0.1 mg and 100 mg may be used. In one embodiment, a daily dosage of between 1 mg and 100 mg may be used.

In other embodiments, the candidate dosage is administered every week with the dosage ranging from about 0.01 mg to about 2000 mg or more, depending on the factors mentioned above. For example, weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used. In one embodiment, a weekly dosage of between 0.01 mg and 0.1 mg may be used. In one embodiment, a weekly dosage of between 0.01 mg and 100 mg may be used. In one embodiment, a weekly dosage of between 0.01 mg and 1 mg may be used. In one embodiment, a weekly dosage of between 0.1 mg and 100 mg may be used. In one embodiment, a weekly dosage of between 1 mg and 100 mg may be used.

In other embodiments, the candidate dosage is administered every two weeks with the dosage ranging from about 0.01 mg to about 2000 mg or more, depending on the factors mentioned above. For example, bi-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used. In one embodiment, a bi-weekly dosage of between 0.01 mg and 0.1 mg may be used. In one embodiment, a bi-weekly dosage of between 0.01 mg and 100 mg may be used. In one embodiment, a bi-weekly dosage of between 0.01 mg and 1 mg may be used. In one embodiment, a bi-weekly dosage of between 0.1 mg and 100 mg may be used. In one embodiment, a bi-weekly dosage of between 1 mg and 100 mg may be used.

In other embodiments, the candidate dosage is administered every three weeks with the dosage ranging from about 0.01 mg to about 2500 mg or more, depending on the factors mentioned above. For example, tri-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or about 2500 mg may be used. In one embodiment, a tri-weekly dosage of between 0.01 mg and 0.1 mg may be used. In one embodiment, a tri-weekly dosage of between 0.01 mg and 100 mg may be used. In one embodiment, a tri-weekly dosage of between 0.01 mg and 1 mg may be used. In one embodiment, a tri-weekly dosage of between 0.1 mg and 100 mg may be used. In one embodiment, a tri-weekly dosage of between 1 mg and 100 mg may be used.

In other embodiments, the candidate dosage is administered every four weeks or month with the dosage ranging from about 0.01 mg to about 3000 mg or more, depending on the factors mentioned above. For example, monthly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500, about 2600 mg, about 2700 mg, about 2800 mg, about 2900 mg, or about 3000 mg may be used. In one embodiment, a monthly dosage of between 0.01 mg and 0.1 mg may be used. In one embodiment, a monthly dosage of between 0.01 mg and 100 mg may be used. In one embodiment, a monthly dosage of between 0.01 mg and 1 mg may be used. In one embodiment, a monthly dosage of between 0.1 mg and 100 mg may be used. In one embodiment, a monthly dosage of between 1 mg and 100 mg may be used.

Other dosage regimens may also be useful, depending on the pattern of pharmacokinetic decay that the practitioner wishes to achieve. In one embodiment, the antibody of the present invention is administered in an initial priming dose followed by a higher and/or continuous, substantially constant dosage. In some embodiments, dosing from one to four times a week is contemplated. In other embodiments, dosing once a month or once every other month or every three months is contemplated. The progress of this therapy is easily monitored by conventional techniques and assays. The dosing regimen can vary over time.

For the purpose of the present invention, the appropriate dosage of an antibody (e.g., one or more selected from the group consisting of anti-IL27RA, gp130 and anti-IL27A/gp130 antibodies) will depend on the antibody or compositions thereof employed, the type and severity of symptoms to be treated, whether the agent is administered for therapeutic purposes, previous therapy, the patient's clinical history and response to the agent, the patient's clearance rate for the administered agent, and the discretion of the attending physician. Typically, the clinician will administer an antibody until a dosage is reached that achieves the desired result. Dose and/or frequency can vary over course of treatment. Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of symptoms. Alternatively, sustained continuous release formulations of antibodies may be appropriate. Various formulations and devices for achieving sustained release are known in the art.

In one embodiment, dosages for an antibody (e.g., one or more selected from the group consisting of anti-IL27RA, gp130 and anti-IL27A/gp130 antibodies) may be determined empirically in individuals who have been given one or more administration(s) of the antibody. Individuals are given incremental dosages of an antibody. To assess efficacy, an indicator of the disease can be followed.

In some embodiments, an antibody provided herein (e.g., one or more selected from the group consisting of anti-IL27RA, gp130 and anti-IL27A/gp130 antibodies) may be administered to a subject that has previously received one or more antibodies selected from the group consisting of anti-IL27RA, gp130 and anti-IL27A/gp130 antibodies therapeutic for treatment of a disease. In some embodiments, an antibody provided herein may be an administered to a subject that has previously received an antibody selected from the group consisting of anti-IL27RA, gp130 and anti-IL27A/gp130 antibodies therapeutic for treatment of a disease, and for which the previous anti-IL27RA, gp130 and anti-IL27A/gp130 antibody therapeutic is of limited or no efficacy in the subject (e.g. for which the subject's disease is resistant to treatment with the prior therapeutic).

Administration of an antibody in accordance with the method in the present invention can be continuous or intermittent, depending, for example, upon the recipient's physiological condition, whether the purpose of the administration is therapeutic or prophylactic, and other factors known to skilled practitioners. The administration of an antibody may be less Therapeutic formulations of the antibody used in accordance with the present invention are prepared for storage by mixing an antibody having the desired degree of purity with optional pharmaceutically acceptable carriers, excipients or stabilizers (Remington, The Science and Practice of Pharmacy 21st Ed. Mack Publishing, 2005), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). entially continuous over a preselected period of time or may be in a series of spaced doses.

### Kits

Another aspect of the invention provides kits comprising the antibody of the invention or pharmaceutical compositions comprising the antibody. A kit may include, in addition to the antibody of the invention or pharmaceutical composition thereof, diagnostic or therapeutic agents. A kit may also include instructions for use in a diagnostic or therapeutic method. In some embodiments, the kit includes the antibody or a pharmaceutical composition thereof and a diagnostic agent. In other embodiments, the kit includes the antibody or a pharmaceutical composition thereof and one or more therapeutic agents.

In yet another embodiment, the invention comprises kits that are suitable for use in performing the methods of treatment described herein. In one embodiment, the kit contains a first dosage form comprising one or more of the antibodies of the invention in quantities sufficient to carry out the methods of the invention. In another embodiment, the kit comprises one or more antibodies of the invention in quantities sufficient to carry out the methods of the invention and at least a first container for a first dosage and a second container for a second dosage.

A further aspect of the invention is a kit comprising one or more selected from the group consisting of anti-IL27RA, anti-gp130 and anti-IL27RA/gp130 antibodies as disclosed herein above and instructions for use in accordance with any of the methods of the invention described herein. Generally, these instructions comprise a description of administration one or more selected from the group consisting of anti-IL27RA, anti-gp130 and anti-IL27RA/gp130 antibodies for the above-described therapeutic treatments.

A further aspect of the invention is a kit comprising the anti-IL27RA/gp130 antibodies as disclosed herein above and instructions for use in accordance with any of the methods of the invention described herein.

The following examples of specific aspects for carrying out the present invention are offered for illustrative purposes only, and are not intended to limit the scope of the present invention in any way

The foregoing description and following Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the teachings herein and the claimed disclosure below. The following examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

### Biological Deposits

Representative materials of the present invention were deposited in the American Type Culture Collection, 10801 University Boulevard, Manassas, VA 20110-2209, USA, on Dec. 17, 2021.

Vector “mAb-4894_IL267RA VH” having ATCC Accession No. PTA-127622 comprises a DNA insert encoding the “mAb-4894_IL267RA VH”. Vector “mAb-4894_IL267RA VL” having ATCC Accession No. PTA-127623 comprises a DNA insert encoding the “mAb-4894_IL267RA VL”. Vector “mAb-4894_IL267RA HC” having ATCC Accession No. PTA127626 comprises a DNA insert encoding the “mAb-4894_IL267RA HC”. Vector “mAb-4894_IL267RA LC” having ATCC Accession No. PTA-127627 comprises a DNA insert encoding the “mAb-4894_IL267RA LC”.

Vector “mAb-4894 gp130 VH” having ATCC Accession No. PTA-127624 comprises a DNA insert encoding the “mAb-4894 gp130VH”. Vector “mAb-4894 gp130VL” having ATCC Accession No. PTA-127625 comprises a DNA insert encoding the “mAb-4894 gp130 VL”. Vector “mAb-4894 gp130 HC” having ATCC Accession No. PTA127628 comprises a DNA insert encoding the “mAb-4894 gp130 HC”. Vector “mAb-4894 gp130 LC” having ATCC Accession No. PTA-127629 comprises a DNA insert encoding the “mAb-4894 gp130 LC”.

| Description | Antibody | ATCC Accession No. |
|---|---|---|
| IL27RA-VH | mAb-4894 IL267RA VH | PTA-127622 |
| IL27RA-VL | mAb-4894 IL267RA VL | PTA-127623 |
| gp130-VH | mAb-4894 gp130 VH | PTA-127624 |
| gp130-VL | mAb-4894 gp130 VL | PTA-127625 |
| IL27RA-HC | mAb-4894 IL267RA HC | PTA-127626 |
| IL27RA-LC | mAb-4894 IL267RA LC | PTA-127627 |
| gp130-HC | mAb-4894 gp130 HC | PTA-127628 |
| gp130-LC | mAb-4894 gp130 LC | PTA-127629 |

The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions; the materials will be promptly replaced on notification with another of the same. Availability of the deposited material is not to be construed as a license to practice the invention in contravention of the rights granted under the authority of any government in accordance with its patent laws.

The disclosure provides for a polynucleotide encoding a IL27RA-VH encoded by the plasmid deposited at the ATCC and having ATCC Accession No. PTA-127622.

The disclosure provides for a polynucleotide encoding a IL27RA-VL encoded by the plasmid deposited at the ATCC and having ATCC Accession No. PTA-127623.

The disclosure provides for a polynucleotide encoding a IL27RA-VH encoded by the plasmid deposited at the ATCC and having ATCC Accession No. PTA-127622, and a IL27RA—VL sequence encoded by the plasmid deposited at the ATCC and having ATCC Accession No. PTA-127623.

The disclosure provides for a polynucleotide encoding a IL27RA-HC encoded by the plasmid deposited at the ATCC and having ATCC Accession No. PTA-127626.

The disclosure provides for a polynucleotide encoding a IL27RA-LC encoded by the plasmid deposited at the ATCC and having ATCC Accession No. PTA-127627;

The disclosure provides for a polynucleotide encoding a IL27RA-HC encoded by the plasmid deposited at the ATCC and having ATCC Accession No. PTA-127626, and a IL27RA-LC sequence encoded by the plasmid deposited at the ATCC and having ATCC Accession No. PTA-127627.

The disclosure provides for a polynucleotide encoding a gp130-VH encoded by the plasmid deposited at the ATCC and having ATCC Accession No. PTA-127624.

The disclosure provides for a polynucleotide encoding a gp130-VL encoded by the plasmid deposited at the ATCC and having ATCC Accession No. PTA-127625.

The disclosure provides for a polynucleotide encoding a gp130-VH encoded by the plasmid deposited at the ATCC and having ATCC Accession No. PTA-127624 and a gp130-VL sequence encoded by the plasmid deposited at the ATCC and having ATCC Accession No. PTA-127625.

The disclosure provides for a polynucleotide encoding a gp130-HC encoded by the plasmid deposited at the ATCC and having ATCC Accession No. PTA-127628.

The disclosure provides for a polynucleotide encoding a gp130-LC encoded by the plasmid deposited at the ATCC and having ATCC Accession No. PTA-127629.

The disclosure provides for a polynucleotide encoding a gp130-VH encoded by the plasmid deposited at the ATCC and having ATCC Accession No. PTA-127628, and a gp130-LC sequence encoded by the plasmid deposited at the ATCC and having ATCC Accession No. PTA-127629.

The disclosure provides for an isolated antibody that specifically binds to IL27RA, comprising a heavy chain variable region (IL27RA-VH) encoded by the plasmid deposited at the ATCC and having ATCC Accession No. PTA-127622.

The disclosure provides for an isolated antibody that specifically binds to IL27RA, comprising a light chain variable region (IL27RA-VL) encoded by the plasmid deposited at the ATCC and having ATCC Accession No. PTA-127623.

The disclosure provides for an isolated antibody that specifically binds to IL27RA, comprising a heavy chain variable region (IL27RA-VH) encoded by the plasmid deposited at the ATCC and having ATCC Accession No. PTA-127622, and a light chain variable region (IL27RA-VL) sequence encoded by the plasmid deposited at the ATCC and having ATCC Accession No. PTA-127623.

The disclosure provides for an isolated antibody that specifically binds to IL27RA, comprising a heavy chain (IL27RA-HC) encoded by the plasmid deposited at the ATCC and having ATCC Accession No. PTA-127626.

The disclosure provides for an isolated antibody that specifically binds to IL27RA, comprising a light chain (IL27RA-LC) encoded by the plasmid deposited at the ATCC and having ATCC Accession No. PTA-127627.

The disclosure provides for an isolated antibody that specifically binds to IL27RA, comprising a heavy chain (IL27RA-HC) encoded by the plasmid deposited at the ATCC and having ATCC Accession No. PTA-127626, and a light chain (IL27RA-LC) sequence encoded by the plasmid deposited at the ATCC and having ATCC Accession No. PTA-127627.

The disclosure provides for an isolated antibody that specifically binds to gp130, comprising a heavy chain variable region (gp130-VH) encoded by the plasmid deposited at the ATCC and having ATCC Accession No. PTA-127624.

The disclosure provides for an isolated antibody that specifically binds to gp130, comprising a light chain variable region (gp130-VL) encoded by the plasmid deposited at the ATCC and having ATCC Accession No. PTA-127625.

The disclosure provides for an isolated antibody that specifically binds to gp130, comprising a heavy chain variable region (gp130-VH) encoded by the plasmid deposited at the ATCC and having ATCC Accession No. PTA-127624, and a light chain variable region (gp130-VL) sequence encoded by the plasmid deposited at the ATCC and having ATCC Accession No. PTA-127625.

The disclosure provides for an isolated antibody that specifically binds to gp130, comprising a heavy chain (gp130-HC) encoded by the plasmid deposited at the ATCC and having ATCC Accession No. PTA-127628.

The disclosure provides for an isolated antibody that specifically binds to gp130, comprising a light chain (gp130-LC) encoded by the plasmid deposited at the ATCC and having ATCC Accession No. PTA-127629.

The disclosure provides for an isolated antibody that specifically binds to gp130, comprising a heavy chain (gp130-VH) encoded by the plasmid deposited at the ATCC and having ATCC Accession No. PTA-127628, and a light chain (gp130-LC) sequence encoded by the plasmid deposited at the ATCC and having ATCC Accession No. PTA-127629.

The disclosure provides for an isolated antibody that specifically binds to IL27RA and gp130, comprising a heavy chain variable region (IL27RA-VH) encoded by the plasmid deposited at the ATCC and having ATCC Accession No. PTA-127622, and a light chain variable region (IL27RA-VL) sequence encoded by the plasmid deposited at the ATCC and having ATCC Accession No. PTA-127623, and further comprising a heavy chain variable region (gp130-VH) encoded by the plasmid deposited at the ATCC and having ATCC Accession No. PTA-127624, and a light chain variable region (gp130-VL) sequence encoded by the plasmid deposited at the ATCC and having ATCC Accession No. PTA-127625.

The disclosure provides for an isolated antibody that specifically binds to IL27R and gp130, comprising a heavy chain (IL27RA-HC) encoded by the plasmid deposited at the ATCC and having ATCC Accession No. PTA-127626, and a light chain (IL27RA-LC) sequence encoded by the plasmid deposited at the ATCC and having ATCC Accession No. PTA-127627, and further comprising a heavy chain (gp130-VH) encoded by the plasmid deposited at the ATCC and having ATCC Accession No. PTA-127628, and a light chain (gp130-LC) sequence encoded by the plasmid deposited at the ATCC and having ATCC Accession No. PTA-127629.

### EXAMPLES

In order that this invention may be better understood, the following examples are set forth. These examples are for purposes of illustration only and are not to be construed as limiting the scope of the invention in any manner.

### Example 1 Generation of gp130/IL27a/iI27 Recombinant Proteins

### Generation of Recombinant Antigens for Immunization and Hybridoma Screening

Human gp130 and IL27RA receptor extra-cellular domain (ECD) complex-Fc fusion (huGP130_IL27RA Knob and Hole huFc) was generated by transfection of 2 expression plasmids, one encoding the hugp130 ECD fused with human Fc with a knob mutation in CH3 region (VEC-40821: CID1452-IgVHSS_huGP130_ECD_TEV_knobFc_2×ST), and the other encoding IL27RA ECD fused with human Fc with the hole mutation in its CH3 region (VEC-40820: CID1451-IgVHSS_huIL27R_ECD_TEV_holeFc_Flag). The knob & hole mutations in the Fc region facilitate the efficient formation of heterodimeric complex between the gp130 and the IL27RA Fc fusions via the Knob and Hole interaction within the respective Fc's.

Expi293F™ cells (Gibco A14527) were propagated in expression medium (Gibco A14351) and non-baffled shake flasks at 8.0% CO2, 36° C. rotating at 120 RPMs in Kuhner ISF1-X incubators. One day prior to transfection cell culture was diluted to 1.6E6/ml in non-baffled shake flasks or Sartorius Wave bag. If necessary, on day of transfection the cell culture was adjusted to 3.0E6/ml. Immediately prior to transfection expression plasmid DNA (1.3 mg per 1 L cell culture) and polyethylenimine (2.6 mg per 1 L cell culture, Polyscience, 24765) were diluted separately into OptiMEM (Gibco 31985-070) then 0.2 uM filtered. Following a 5-minute incubation at room temperature the diluted reagents were combined incubated for an additional 6 minutes at room temperature then added to cell culture. 2.5 hours after transfection valporic acid (Sigma P4543) was added to a final concentration of 3 mM. 120 hours after transfection cell culture was transferred to sterile 1 L Nalgene bottles and centrifuged for 5-10 min at 1-3,000×g. Clarified conditioned media was 0.8/0.2 uM depth filtered (Sartopore 2 XLG) then either processed further or stored frozen at −20 C.

Conditioned media was batch bound to protein A resin (MabSelect SuRe, Cytiva) equilibrated and washed in Buffer A (PBS with 20 mM Imidazole). The bound protein was eluted using Buffer B (150 mM Glycine pH 3.5, 40 mM NaCl) that was immediately neutralized with 10% 1 M Tris-HCl pH 8.0. Elution fractions containing the protein of interest were pooled, concentrated using a 10K MWCO Amicon Ultra concentrator (Millipore), and applied to a HiLoad Superdex S200 16/60 pg column (Cytiva)

In addition, the expression and purification of recombinant cyno huGP130_IL27RA Knob and Hole Fc protein was performed in a similar way.

### Generation of Recombinant IL27 Ligand for Activity Assays

There are two subunits for the ligand IL27, p28 and Ebi3. Two forms of the ligand complex were generated:

### 1. MonoFc Format:

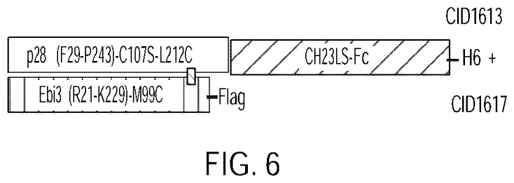

IL27 huIL27p28_C107S_L212C_CH23LSFc_His6/huEBI3_M99C_Flag as shown below in FIG. 6.
IL27 p28 monoFc construct sequence is provided as SEQ ID NO: 49 and EBI3 construct sequence is provided as SEQ ID NO: 50

Expression was performed in similar manner as described for that of antigens, and purification process as, CM was batch bound to protein A resin that had been washed with 15 CV Buffer A (137 mM NaCl, 2.7 mM KCl, 8.1 mM Na2HPO4, 1.47 mM KH2PO4, 20 mM Imidazole). The sample was eluted using a low pH buffer (buffer B: 150 mM Glycine pH 3.5, 40 mM NaCl) and neutralized with buffer C (1M Tris pH8) at a 1:10 V:V ratio (buffer to sample). The different fractions were than run on a gel and those that have the protein were pooled and concentrated using a 10K MWCO Amicon Ultra 4 tubes. Final purification was performed using a HiLoad Superdex column equilibrated with PBS+1MNaCl at 4° C. Collected Fractions were run on the gel and those that contain the protein were pooled. An analytical SEC was run on the final sample with the equilibration buffer either PBS or PBS with 1M NaCl.

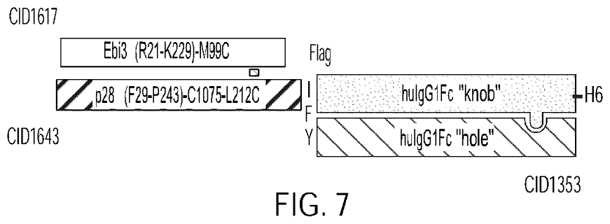

Heterodimeric Knob and Hole format:
IL27_huIL27A_C107S_L212C_TEV_knobFc_His6/huEBI3_M99C_Flag/Hole empty as shown in FIG. 7.

EBI3 provided as SEQ ID NO: 50. IL27 p28 Fc Knob [Knob mutations Y349C-T366W] provided as SEQ ID NO: 51. Fc Hole (Empty) [Hole mutations 5354C-T3665-L368A-Y407V] provided as SEQ ID NO: 52.

Expression was performed in similar manner as described for that of antigens, and purification process as, CM was batch bound to protein A resin that had been washed with 15 CV Buffer A (PBS). The sample was eluted using a low pH buffer (buffer B, 150 mM Glycine pH 3.5, 40 mM NaCl) and neutralized with buffer C (1M Tris pH8) at a 1:10 V:V ratio (buffer to sample). The different fractions were than run on a gel and those that have the protein were pooled and concentrated using a 10K MWCO Amicon Ultra 4 tubes. The protein was than batch bound to anti-flag resin (10 ml) that have been already washed with PBS. After an overnight batch binding the protein was eluted with low pH buffer B and neutralized with buffer C (1:10 V:V ration, buffer to sample). The different fractions were than run on a gel and those that have the protein were pooled and concentrated using a 10K MWCO Amicon Ultra 4 tubes Final purification was performed using a HiLoad Superdex column equilibrated with PBS at 4° C. Collected Fractions were run on the gel and those that contain the protein were pooled and stored at −80 C.

### Example 2 Derivation of IL27RA and Gp130 Binding Domains Via Hybridoma Approach

Generation and Isolation of Monoclonal Antibodies from Humanized Mouse that Bind Human and Cyno gp130

AlivaMab κλ mice were immunized once per week with soluble hIL-27RA_gp130 Knob and Hole hFc protein combined with a mixture of TLR agonists as an adjuvant. They were immunized according to the following schedule shown in Table 1 below:

TABLE 1
mouse immunization schedule

| Day of protocol | Immunogen | Site of immunization |
|---|---|---|
| 0 | 50 ug soluble hIL-27Ra_gp130 K/H-hFc protein + | Base of the neck |
| | TLR agonist cocktail | Base of the tail |
| | | Hock |
| 7 | 50 ug soluble hIL-27Ra_gp130 K/H-hFc protein + | Sub-cutaneous |
| | TLR agonist cocktail | Intra-peritoneal |
| 14 | 50 ug soluble hIL-27Ra_gp130 K/H-hFc protein + | Base of the neck |
| | TLR agonist cocktail | Base of the tail |
| | | Hock |
| 21 | 50 ug soluble hIL-27Ra_gp130 K/H-hFc protein + | Sub-cutaneous |
| | TLR agonist cocktail | Intra-peritoneal |
| 56 | 50 ug soluble hIL-27Ra_gp130 K/H-hFc protein + | Base of the neck |
| | TLR agonist cocktail | Base of the tail |
| | | Hock |
| 62 (two mice) | 50 ug soluble hIL-27Ra_gp130 K/H-hFc protein | Tail vein |

The TLR agonist mixture consisted of the following components:

TABLE 2
TLR against mixture

| Compo- | | Stock | | |
|---|---|---|---|---|
| nent | Catalogue # | concentration | Amount/mouse | Volume/mouse |
| MPLA-SM | InvivoGen | 1 mg/mL | 10 ug | 10 uL |
| | vac-mpla | | | |
| ODN1826 | InvivoGen | 2 mg/mL | 20 ug | 10 uL |
| | vac-1826-1 | | | |
| R848 | InvivoGen | 1 mg/mL | 10 ug | 10 uL |
| | vac-r848 | | | |
| Poly(I:C) | InvivoGen | 1 mg/mL | 10 ug | 10 uL |
| | vac-pic | | | |

The protein was combined with the indicated volume of TLR agonist cocktail (40 uL per mouse) and injected.

On day 28, immune sera were collected. They were screened for binding to hIL-27RA_gp130 Knob and Hole hFc and to a control hFc-tagged protein in a protein ELISA format. Sera were also screened by flow cytometry for binding to HEK293 cells, which express the native IL27RA/gp130 receptor, and to CHO cells expressing recombinant IL-27RA/gp130. Finally, sera were screened for IL-27RA/gp130 agonist activity in a pSTAT3 HTRF assay. Two mice with the best titers received an additional boost on day 56 and a final boost on day 62 of 50 ug of immunogen without adjuvant. Four days later the mice were euthanized and their splenocytes and lymph node cells were fused with Sp2mIL6 mouse myeloma cells by electrofusion.

Hybridomas were screened for binding to hIL-27RA_gp130 Knob and Hole hFc and to a control hFc-tagged protein in a protein ELISA format. They were also tested for binding by flow cytometry to CHO parental cells, CHO cells expressing the IL-27RA chain only, CHO cells expressing gp130 only, and CHO cells expressing the full IL-27RA-gp130 receptor. Prioritized hybridomas generating antibodies that employed the kappa light chain and which bound to CHO-gp130 and CHO IL-27RA_gp130 cells were identified as gp130-specific antibodies and subjected to VH-VL molecular cloning.

Briefly, cDNA was generated from purified RNA using the SMARTer IIA oligonucleotide (Clontech) and oligo(dT)20. cDNA was then amplified using RACE PCR. To amplify the heavy chain, the oligo SMART 2ndF (GTGGTATCAACGCAGAGTACGCG) (SEQ ID No: 55) was employed as the forward primer, and GGGTGCCAGGGGGAAGACSGA (SEQ ID NO: 56) specific for mouse IgG as the reverse primer. To amplify the kappa light chain, the oligo SMART 2ndF (GTGGTATCAACGCAGAGTACGCG)—SEQ ID No: 53—was also used as the forward primer, and the kappa specific primer GAAGATGAAGACAGATGGTGCAGCCAC—SEQ ID No: 54—the reverse. The VH amplicons were then cloned via infusion cloning into the pTT5-hIgG_EFN_EEE_Flag (SAPI, SMART) vector and the VL amplicons into the pTT5-hKappa (SAPI, SMART) vector. Thirteen unique paired sequences were identified and selected for further characterization (clones −2194, −2211, −2191, −2203, −2200, −2207, −2183, −2214, −2202, −2187, −2189, −2199 and −2201). Clone −2187, derived from hybridoma 13B10, provided the lead binding domain.

Isolation of Rat Monoclonal Antibodies that Bind Human and Cynomolgus IL27RA

Sprague-Dawley rats were immunized with 12 IP injections of 20 ug of soluble hIL-27Ra_gp130 Knob and Hole hFc protein emulsified in Ribi adjuvant (Sigma S6322). The first 9 immunizations were carried out twice per week; the remaining 3 immunizations were once per week. Immune sera were screened for binding to HEK293 cells and HEK293 cells rendered deficient for native IL-27Ra/gp130 expression by CRISPR knockdown. They were also tested for agonist activity in a pSTAT3 HTRF assay. One rat with the best titer received a final boost of 20 ug of immunogen without adjuvant. Seven days later the rat was euthanized and its splenocytes were fused with the P3X mouse myeloma cells by electrofusion.

Hybridomas were screened for binding to hIL-27RA_gp130 Knob and Hole hFc and to a control hFc-tagged protein in a protein ELISA format, as well as by flow cytometry for binding to HEK293 cells and HEK293_IL-27RA/gp130 CRISPR knockdown cells. Prioritized hybridomas were subjected to limiting dilution subcloning. Subclones were tested for binding to hIL-27RA_gp130 Knob and Hole hFc by ELISA and by flow cytometry to CHO parental cells, CHO cells expressing the IL-27RA chain only, CHO cells expressing gp130 only, and CHO cells expressing the full IL-27RA-gp130 receptor. 23 hybridomas expressing antibodies specific to the IL-27RA chain were subjected to VH-VL molecular cloning. Clone −0917, derived from hybridoma 105_A6-10-7, provided the lead binding domain.

### Screening Antibody Pairs to Identify IL27RA/Gp130 Agonists

Screening of the antibodies raised against IL27RA or against gp130 from hybridoma described in Example 2 failed to identify single mAbs that could agonize IL-27RA/gp130 overexpressed in CHO cells as measured by STAT3 phosphorylation. Therefore, a matrix of antibodies was tested in bispecific format to identify agonists of the heterodimer.

A set of 87 anti-IL27RA antibodies, representing 44 distinct CDR H3 families and 72 unique CDR H3-CDR L3 sequences and a mixture of IL-27 functional inhibitors and non-inhibitors, was collected from the hybridoma screen described in Example 2 and from screening phage display libraries (data not shown). A set of 89 anti-gp130 antibodies, representing 76 distinct CDR H3 families and 89 unique CDR H3-CDR L3 sequences and a mixture of IL-27 functional inhibitors and non-inhibitors, was collected from similar sources. Bispecific antibodies were generated by cloning anti-IL-27RA antibodies into a human IgG1 vector (“RRR”) carrying engineered arginine residues D221R, P228R, and K409R (EU numbering) and cloning anti-gp130 antibodies into a human IgG1 vector (“EEE”) carrying engineered glutamic acid residues D221E, P228E, and L368E (EU numbering). Under mild reduction and reoxidation, a mixture of antibodies carrying the RRR and EEE mutations will preferentially form RRR-EEE heterodimers (Strop et al, 2012). This redox procedure was miniaturized so that bispecific antibodies could be formed from mixtures of 20 micrograms of each starting antibodies, incubated first with reduced 1 mM glutahione (GSH) for 1 hr at 37 C, and subsequently reoxidized with 1 mM glutathione disulfide (GSSG). The resulting material was shown to be compatible with cell- and whole-blood assays for IL-27R activity, including phosphorylation of STAT3 in CHO cells overexpressing IL-27RA and gp130 (described in Example 2), and phosphorylation of STAT3 and STAT1 in CD3+ T cells in human whole blood (described in Example 8).

A total of 2156 bispecific IgGs was generated. Of these, 50 showed agonism in the CHO pSTAT3 assay, and (among 29 strong agonists tested), 12 showed robust agonism of pSTAT 1 in CD3+ T cells in human whole blood (data not shown). These observations were reproduced when these 12 bispecific antibodies were produced at the multi-milligram scale and purified away from redox buffer and any remaining parental antibodies. These 12 represented combinations of 4 anti-gp130 antibodies (Ab 2187 and three unrelated antibodies) with 5 anti-IL-27RA antibodies (Ab 917 and four unrelated antibodies), each with unique VH and VL sequences. Within this set of antibodies, two “clusters” were evident: any member of one set of three anti-gp130 antibodies (including Ab-2187) could pair with any member of one set of three anti-IL-27RA antibodies (including Ab-0917) to form active bispecific agonists. The most potent activity was observed from the bispecific pair of Ab-0917 and Ab-2187, and these were chosen as the lead binding domains for humanization and optimization (Example 3).

### Example 3 Humanization and Optimization of Binding Domains

### Humanization and Optimization of Anti-Gp130 Binding Domain

Anti-gp130 clone 2187's heavy chain variable domain was subcloned into expression vector containing non-tagged human Fc and the protein thus produced was renamed as clone 2246, which will be referred to as the parental clone for the description of humanization and optimization.

2246 binding domain had human framework IGHV4-4*07_IGHJ4*03 for VH and IGKV3-20*01_IGKJ4*01 for VL. IGHV4-4*07_IGHJ4*03 is a less optimal framework than preferred frameworks in terms of biophysical properties and manufacturabilities. In addition, an in-silico T-cell epitope analysis revealed an extremely poor Epivax ISPRI immunogenicity propensity score for the 2246 VH sequence (+6.2, compared with the preferred score of <−50. While this high score was primarily modulated by germline epitopes rather than non-germline epitopes, this score still indicates an increased immunogenicity risk unless associated with one of the preferred germlines, which have been empirically de-risked in multiple late-stage programs. Re-humanization was therefore performed to graft the VH CDRs into another framework IGHV1-69*01, with 6 back mutations (A24V, M481, 169M, A71V, E73T, A78F) (Pfabat numbering) to recover binding activity. 2246 VL CDRs were also grafted into framework IGKV1-39*01 with no back-mutation. The re-humanized clone 4247 fully retained the binding activity of parental clone 2246. However, it still had 3 non-germline T cell epitopes in CDRs, 2 in VH and 1 in VL, as well as a moderately high immunogenicity score of −40.05 (current standard, Pfizer TReg-Adjusted v1.00); it also had an N-linked glycosylation site in CDR-H2.

Before the availability of a co-crystal structure of the 2246 series, a set of 96 point mutations, predicted to reduce T-cell epitope content and eliminate the N-linked glycosylation site in H2 with minimal impact on stability, were designed, synthesized, and characterized for retention of gp130 binding. No mutation was able to remove the glycosylation site in H2 without significant loss of binding activity. However, this liability was determined to be of low risk and experimentally demonstrated to be unoccupied. Therefore, no further efforts to remove it was made. When the co-crystal structure of gp130/Fab3754 (a rehumanization variant of parental 2246) was solved, a second set of mutations were also designed to potentially enhance binding activity without adding new T-cell epitopes, to hedge against the loss of binding activity. 20 affinity mutations were combined with 9 binding-verified epitope-removal mutations to generate a set of 54 VH chains and 7 VL chains, which were matrixed to create 378 antibody optimization variants. These mutant variants were screened for retention of gp130 binding activities and 10 constructs that did not reduce binding activity or molecular properties were then advanced to the next round of screening triage, along with 2 new combination variants. Simultaneously, 20 back-mutation variants of 4247 were evaluated for reduced in silico immunogenicity risk and retention of gp130-binding; 3 of these variants, plus the unmodified 4247, were advanced to the next screening round. In the final round of screening, the 12 optimized CDR constructs were crossed with the 4 framework variants and assayed for gp130-binding and molecular properties. The final resulting IgG molecule, clone 4574, incorporated 5 total mutations: 2 mutations into the VH CDRs, 2 into the VL CDRs, and 1 into the FW-H. Mutations S(H54)T, S(H65)D, F(H78)H, and S(L52)E (Pfabat numbering) reduce T-cell epitope content, while mutation S(L94)Y appears to compensate for small losses in gp130 binding. Clone 4574 is devoid of predicted non-germline epitope and other tier 1 sequence liabilities.

### Humanization and Optimization of Anti-IL27RA Binding Domain

Anti-IL27RA clone 0917's heavy chain variable domain was subcloned into expression vector containing non-tagged human Fc and the protein thus produced was renamed as clone 2255, which will be referred to as the parental clone for the description of humanization and optimization.

For humanization, 2255's VH CDRs were grafted into framework IGHV3-7*01 with 2 back mutations V481 & A49G (Pfabat numbering); and VL CDRs into IGKV1-39*01 with 3 backmutations L46R, L47V, Y49F (Pfabat numbering). The resultant humanized molecule fully retained the binding activity of parental clone 2255. However, it had 6 non-germline T cell epitopes spread between all 3 heavy-chain CDRs, 2 additional non-germline T cell epitopes in the L2, 1 potential tier 1 deamidation site in CDR-L1, and very high polyreactivity scores (DNA 26, insulin 14 while desired score <5). A limited screen of 16 CDR variants aimed at reducing the T-cell epitope content revealed that the K(L53)G mutation (clone 4207) significantly reduced the DNA-binding polyreactivity score, while maintaining much of the parental IL27R-binding activity and removing a T-cell epitope. Additional screens of variants of humanized 2255 with an alternative K(L53)D mutation (˜500 clones) and variants of the homologous humanized 2257 antibody (˜150 clones) identified several mutations with putative immunogenicity- or DNA-affinity-reducing benefits that were likely to be tolerated as mutations to 4207. The results of these screens were combined with a structural analysis of the newly available co-crystal structure of IL27R/Fab2255 to generate a set of 48 single-CDR variants of 4207, each containing 1-3 mutations, predicted to reduce T-cell epitope content and/or polyreactivity with minimal impact on stability and affinity, or to potentially enhance binding activity, in case it is needed to compensate for the loss of binding activity from liability-removing mutations. Mutant variants were screened for retention of IL27R binding and DNA polyreactivity, and the mutations from −10 variants with favorable profiles were recombined into 80 VH and 9 VL sequences, which were in turn matrixed to create a final set of 720 antibody optimization variants. The optimized variant set was similarly synthesized, screened, and triaged to identify the final resulting IgG molecule, clone 4701. Clone 4701 incorporated 5 mutations into the VH CDRs, and an additional mutation into the L2. Mutations N(H35)Q, I(H51)T, I(H57E), and K(L53)G (Pfabat numbering) each reduce T-cell epitope content, and all appear to contribute at least somewhat to reduced DNA-binding; mutation S(H30)E appears to reduce DNA-binding, while A(H96)K appears to compensate for IL-27R-binding activity lost through the other mutations. No tolerated mutations were identified that remove the potential NS deamination site in CDR-L1, however, this deamination liability was de-risked in expanded stage I assessment and therefore was not further pursued. Clone 4701 was devoid of predicted non-germline epitope, and retains just one de-risked tier 1 sequence liability in CDR-L1. It also had much reduced polyreactivity scores, DNA 10-13 and insulin 5-7. When combined with the optimized anti-gp130 arm (4574) in a bispecific molecule, the polyreactivity scores were further reduced to <5, the acceptable range.

### Example 4 Polyreactivity/Non-Specific Interaction Assessment

Final optimized binding domains for both gp130 and IL27Ra were assessed in molecular assessment suit of biophysical properties as monospecific homodimeric IgGs (clone 4574 for anti-gp130 arm and clone 4701 for anti-IL27RA arm), and also as the final bispecific molecule mAb-4894. 3 types of assays were performed to assess the polyreactivity/non-specific interaction propensity as described below.

### DNA and Insulin ELISA

384-well ELISA plates (Nunc Maxisorp) were coated overnight at 4° C. with DNA (10 μg/ml) and insulin (5 μg/ml) in PBS pH 7.2. The ELISA, adapted from assays described in Tiller et al., J. Immunol. Methods 329, 112, 2008; U.S. Pat. No. 7,314,622, was carried out on a PerkinElmer Janus liquid handling robot. Wells were washed with water, blocked with 50 μl of Polyreactivity ELISA Buffer (PEB; PBS pH 7.2 containing 0.05% Tween-20, 1 mM EDTA) for 1 hour at room temperature, and rinsed once with 80 μl of water. Test samples at 10 ug/ml (in PBS pH 7.2, 0.05% Tween-20, 1 mM EDTA) were added in quadruplicate to the wells and incubated for 1 h at room temperature. Plates were washed 3 times with 80 μl of water, and 25 μl of 32 ng/ml goat anti-human IgG (Fcγ fragment specific) conjugated to horseradish peroxidase (Jackson ImmunoResearch) in PBS pH 7.2, 0.05% Tween-20, 1 mM EDTA, was added to each well. Plates were incubated for 1 h at room temperature, washed 3 times with 80 μl of water, and 25 μl of TMB substrate (Sigma Aldrich) was added to each well. Reactions were stopped after 6 minutes 45 seconds by adding 25 μl of 0.18 M ortho-phosphoric acid to each well and absorbance was read at 450 nm. DNA- and insulin-binding scores were calculated as the ratio of the ELISA signal of the antibody at 10 μg/ml to the signal of a well containing buffer.

### Affinity Capture Self-Interaction Nanoparticle Spectroscopy (AC-SINS)

Proteins have the potential to interact with themselves, particularly at increased concentrations. This self-interaction can lead to viscosity challenges associated with formulation during drug development, as well as increased risk of clearance. (Avery et al. MAbs. 2018; 10(2): 244-255). The AC-SINS assay measures self-interaction and is used to help predict high viscosity and the potential for poor pharmacokinetic properties.

The AC-SINS assay was standardized in a 384-well format on a Perkin-Elmer Janus liquid handling robot. 20 nm gold nanoparticles (Ted Pella, Inc., #15705) were coated with a mixture of 80% goat anti-human Fc (Jackson ImmunoResearch Laboratories, Inc. #109-005-098) and 20% non-specific goat polyclonal antibodies (Jackson ImmunoResearch Laboratories, Inc. #005-000-003) that were buffer exchanged into 20 mM sodium acetate pH 4.3 and diluted to 0.4 mg/ml. After one hour incubation at room temperature, sites unoccupied on the gold nanoparticles were blocked with thiolated polyethylene glycol (2 kD). The coated nanoparticles were then concentrated 10-fold using a syringe filter and 10 μl were added to 100 μl of test sample at 0.05 mg/ml in PBS pH 7.2. The coated nanoparticles were incubated with the test sample for 2 hrs in a 96-well polypropylene plate and then transferred to a 384-well polystyrene plate and read on a Tecan spectrophotometer. The absorbance was read from 450-650 nm in 2 nm increments, and a Microsoft Excel macro was used to identify the max absorbance, smooth the data, and fit the data using a second-order polynomial. The smoothed max absorbance of the average blank (PBS buffer alone) was subtracted from the smoothed max absorbance of the sample to determine the AC-SINS score.

### Human FcRn Column

Human FcRn affinity column was prepared as previously described in Koch et al., mAbs 5:576-586, 2013 (see below). To determine the human FcRn column retention time, 50 μg of test sample adjusted to pH 5.5 was injected onto a GE High Performance Streptavidin Sepharose column (cat #17-5113-01) coated with in-house expressed and purified biotinylated hFcRn, and a linear pH gradient from pH 5.5 (MES) to 8.8 (Tris) in the presence of 150 mM NaCl was applied for elution of sample. The FcRn column data are reported as a relative retention time where the elution time of sample is subtracted from that of an assay performance control, mAb-A, to normalize variability within assay.

Acceptable low-level risk typical for monoclonal antibodies was determined for the final bispecific molecule mAb-4894 based on results from these of assays as shown in Table 3 below.

TABLE 3
results of polyreactivity/non-specific interaction assessment
Polyreactivity/non-specific interaction assessment

| | H/M/L scores | |
|---|---|---|
| | (DNA/Insulin/AC- | |
| | SINS) | |
| | High score = >8 | |
| | Medium score = 6-8 | Comments e.g. comparison with positive and |
| Assays | Low score = 0-5 | negative controls. |
| DNA binding | HEK (Stage II) = 4.4 | Non-specificity scores for the bispecific are |
| (ELISA) scores | CHO (ETS) = 5.3 | low for HEK/CHO materials and comparable |
| (n of 2, independent | | to well-behaved mAb (assay negative |
| experiments) | | control). The H/M/L score criteria were |
| Insulin binding | HEK (Stage II) = 4.4 | established based on mAbs. High scores for |
| (ELISA) scores | CHO (ETS) = 5.4 | mAbs tend to correlated with unwanted |
| (n of 2, independent | | properties such as off-target binding and fast |
| experiments) | | clearance. Non-specificity in vitro/in vivo |
| AC-SINS scores | HEK (Stage II) = 1.0 | correlation data is limited for multispecifics. |
| (n of 2, independent | CHO (ETS) = 1.2 | Note that the DNA binding score is high for |
| experiments) | | the IL27RA mAb (4701). DNA binding score |
| | | differences (low for the bispecific and high for |
| | | the IL27R mAb) may be attributed to valency. |
| Human FcRn | HEK (Stage II) = 0.02 | FcRn rRT for HEK/CHO materials are |
| chromatography | CHO (ETS) = 0.14 | comparable to well-behaved mAb (assay |
| relative Retention | | negative control). Limited FcRn data for |
| Time (rRT, min) | | multispecifics. |

### Example 5. Large Scale Production of Lead Antibody

Generation of Stable CHO Cell Pools for Large Scale Production of mAb-4894

The two binding arms for the final bispecific molecule mAb-4894 were expressed separately as IgGs (homodimer) with either the EE or RR mutations in its IgG1 Fc domain to facilitate heterodimer formation during a redox reaction performed later in purification. Particularly, the anti-gp130 binding arm with RR mutations in its Fc is named clone anti-gp130 4875 RR (derived from 4574, also referred to as anti gp130-4574), and the anti-IL27RA arm with EE mutations named clone anti-IL27RA 4880 EE (derived from 4701, referred to as IL27RA—4701). The EE/RR mutations in the heavy chain constant region to facilitate heterodimer formation: on the EE side, D221E & L368E (by EU numbering) or D234E & L381E (by Kabat numbering); on the RR side, D221R & K409R (by EU numbering) or D234R & K422R (by Kabat numbering). The EE arm holds the anti-IL27RA binding variable domain and the RR side arm holds the anti-gp130 variable domain. The binding arms of the final bispecific also contained alanine mutations to minimize effector-function: L234A, L235A, G237A (by EU numbering or L247A, L248A, G250A (by Kabat numbering) in accordance with human IgG1.

Expression vectors harboring cDNAs encoding clone anti-gp130 4875 RR and IL27RA 4880 EE were constructed in the SSI system, as pSSI2.0-IL27-4574-2X and pSSI2.0-IL27-4701-2X, respectively. The two expression vectors were each transfected into CHOK1 SV SSI 7876 (HCLIB-53) cells using Neon electroporation (Bio-Rad) to generate two independent stable pools. Selection of transfectants that stably express the relevant antibody proteins utilized glutamine synthesis and blasticidin (Gibco Cell Culture) resistance in CDCHO (Invitrogen) media.

Both pools were then expressed separately using 10 L of CHO cells grown in Pfizer production media. The conditioned media for both homodimers (EE and RR) were harvested on day 12 and processed for purification that'll be described later. (CHO cell lines secreting IL-27R antibody homodimers (designated IL-27R-4880 EE and gp130-4875 RR) were generated using Pfizer's Biotherapeutics Pharmaceutical Sciences (PharmSci) Cell Line Development Laboratory's protocol (CLD_SSI 2.0 v1))

### IL27RA-4880 EE Homodimer Cell Line Generation:

Electroporation of CHOK1 SV SSI 7876 cells (HCLIB-53) was performed at 300V and 900 uF using 5μg of pSS12.0 expression plasmid encoding Anti-IL27RA-4880 EE homodimer antibody (VEC-38718) and 45 μg pFIpE recombinase plasmid (AVEC-25020). Transfections were done in triplicate to generate three independent pools (DX18-1, DX18-2 and DX18-3). The resulting pools were fluid changed 24 hours post transfection into L-Glutamine free media (CDCHO-Invitrogen Cat #10743-029 Lot 2085431. Cultures are monitored on a 3-4-day schedule. After establishment, a small-scale production study was performed by seeding 200 mL volume cultures with 0.3×106 cells/mL in CDCHO completely defined pre-load media (MFR H000002813). On Days 3 through 6, and 10 and 11, the cultures were sampled and fed with 5.4 mL completely defined feed version 6.2A (CDFv6.2A) and 5.0 mL 10%, glucose. On day 7 cultures were sampled and fed with 16.2 mL CDFv6.2A and 15.0 mL 10% glucose. Conditioned medium was harvested from pools DX18-1, DX18-2 and DX18-3 on Day 12 for the crude Protein A titer assessment. Two 10 L working volume controlled reactor was seeded with cell culture containing a pool of pools (DX18-1, DX18-2 and DX18-3 at 1:1:1 ratio DX18-PoP) in 8 L M310 production media (PFP SOI AN LAB 0701: GS AU8 with Spermine 4HCl, 12 mM Asparagine, 12 mM Aspartic Acid, 8 g/L Glucose, +14 mM KCl (M310)**Lot #0A215201026026). The culture was held at a temperature of 36.5° C., rocking at 18 RPM with an angle of 12°. Cultures were fed with 3.4% M391 (EXP M391Lot #0A215201028002) per day (days 3-12) and 2.5% per day 10% glucose (days 4-12). pH controlled to 7.05+/−0.15. Dissolve oxygen set point was 30%. Conditioned medium was harvested on day 12 by filtration through a 30″ 5 μm Pall Profile® II filter (Cat #NP8Y050BP1G) and a 10″ 0.22 μm Pall Support filter (Cat #NP6EKVP1GA).

TABLE 4
Small (200 mL) and Large (10 L) Scale Expression Analysis of IL27R-4880 EE
Homodimer from Stable CHO SSI Pools

| | | Seed | | Peak | Harvest | Harvest | Harvest |
|---|---|---|---|---|---|---|---|
| | Scale | VCD | Harvest | VCD | VCD | % | Titer |
| Pool | (mL) | (cell/mL) | Date | (cell/mL) | (cell/mL) | V | (mg/L) |
| DX18-1 | 200 | 0.3 | Aug 26, | 12.8 | 12.1 | 93.2 | 1489 |
| | | | 2020 | | | | |
| DX18-2 | 200 | 0.3 | Aug 26, | 12.1 | 10.5 | 90.2 | 1460 |
| | | | 2020 | | | | |
| DX18-3 | 200 | 0.3 | Aug 26, | 11.8 | 10.4 | 88.0 | 1257 |
| | | | 2020 | | | | |
| DX18-POP | 2 × 10,000 | 2.1/1.8 | Nov 11, | 29.1/ | 21.7/ | 94.2/ | 971/ |
| | | | 2020 | 30.1 | 29.0 | 98.9 | 1288 |

VCD = Viable Cell Density

### Anti-Gp130-4875 RR Homodimer Cell Line Generation:

Electroporation of CHOK1 SV SSI 7876 cells (HCLIB-53) was performed at 300V and 900 uF using 5μg of pSSI2.0 expression plasmid encoding Anti-gp130-4875 RR homodimer antibody (VEC-38719) and 45 μg pFIpE recombinase plasmid (AVEC-25020). Transfections were done in triplicate to generate three independent pools (DX19-1, DX19-2 and DX19-3). The resulting pools were fluid changed 24 hours post transfection into L-Glutamine free media (CDCHO-Invitrogen Cat #10743-029 Lot 2085431. Cultures are monitored on a 3-4-day schedule. After establishment, a small-scale production study was performed by seeding 200 mL volume cultures with 0.3×106 cells/mL in CDCHO completely defined pre-load media (MFR H000002813). On Days 3 through 6, and 10 and 11, the cultures were sampled and fed with 5.4 mL completely defined feed version 6.2A (CDFv6.2A) and 5.0 mL 10%, glucose. On day 7 cultures were sampled and fed with 16.2 mL CDFv6.2A and 15.0 mL 10% glucose. Conditioned medium was harvested from pools DX19-1, DX19-2 and DX19-3 on Day 12 for the crude Protein A titer assessment. A 10 L working volume controlled reactor was seeded with cell culture containing a pool of pools (DX19-1, DX19-2 and DX19-3 at 1:1:1 ratio DX19-PoP) in 8 L M310 production media (PFP SOI AN LAB 0701: GS AU8 with Spermine 4HCl, 12 mM Asparagine, 12 mM Aspartic Acid, 8 g/L Glucose, +14 mM KCl (M310)**Lot #0A215201026026). The culture was held at a temperature of 36.5° C., rocking at 18 RPM with an angle of 12°. Cultures were fed with 3.4% M391 (EXP M391Lot #0A215201028002) per day (days 3-12) and 2.5% per day 10% glucose (days 4-12). pH controlled to 7.05+/−0.15. Dissolve oxygen set point was 30%. Conditioned medium was harvested on day 12 by filtration through a 30″ 5 μm Pall Profile® II filter (Cat #NP8Y050BP1G) and a 10″ 0.22 μm Pall Support filter (Cat #NP6EKVP1GA).

TABLE 5
Small (200 mL) and Large (25 L) Scale Expression Analysis of IL27R-4875 RR from
Stable CHO SSI Pools

| | | Seed | Peak | Harvest | Harvest | Harvest |
|---|---|---|---|---|---|---|
| | Scale | VCD | VCD | VCD | % | Titer |
| Pool | (mL) | (cell/mL) | (cell/mL) | (cell/mL) | V | (mg/L) |
| DX19-1 | 200 | 0.3 | 13.0 | 12.5 | 91.4 | 1489 |
| DX19-2 | 200 | 0.3 | 12.2 | 10.8 | 87.7 | 1460 |
| DX19-3 | 200 | 0.3 | 11.0 | 9.8 | 94.9 | 1257 |
| DX19-POP | 10,000 | 1.7 | 32.2 | 32.2 | 93.6* | 2590 |

### Redox and Purification of Lead Bispecific Antibody

Conditioned media of EE and RR homodimers were captured separately on MabSelect Sure LX resin on an Akta Avant (GE Healthcare Life Sciences). The redox reaction to produce heterodimers was conducted in vitro. A 1:1 ratio of homodimers and a molar excess of cysteine were incubated together. The post-redox material was purified on a column with Fractogel TMAE Hicap (M) (EMD Millipore) resin equilibrated in 50 mM Tris, pH 8.1 in weak-partitioning mode. Further purification was optimized using a micro-column screen of Ion-exchange (IEX) and hydrophobic interaction chromatography (HIC) resins. The material was diluted 1:1 with 800 mM Sodium Sulfate, 700 mM Sodium Phosphate, 100 mM Tris, pH 7.2, and purified using Butyl HP HIC resin (Cytiva) using a gradient of elution buffer (50 mM Sodium Phosphate, pH 7.2). The final buffer exchange to His/Sucrose buffer (20 mM Histidine, 8.5% sucrose, pH 5.8) utilized a 30 kDa regenerated cellulose membrane (EMD Millipore). Absorbance at 280 nm was used to quantitate protein concentrations, using the calculated absorption coefficient.

The evaluation of % conversion was carried out by HP-HIC (High Performance−Hydrophobic Interaction Chromatography)−the method leveraged the hydrophobicity differences of the two homodimers/components (anti-IL27RA (IL27R-4880 EE) and anti-gp130 (IL27R-4875 RR)) and the final product protein (heterodimer). Samples were ran using a gradient of 1M sodium sulfate, 50 mM Sodium phosphate, pH 7.2 on a ProPac HIC-10 column (Thermo Fisher). HP-SEC was performed by injecting 15 μL of the reaction mixture into a HPLC equipped with a YMC-pack-Diol 200 column and a UV detector (280 nM). The column temperature was set at 25° C. and the flow rate was maintained at 0.3 mL/min using an isocratic elution (buffer 50 mM Sodium Phosphate).

### Example 6 Evaluation of IL27RA/gp130 Binding to Human and Cynomolgus Monkey IL27RA and gp130

### Results

The crossreactivity of mAb-4894 to human and cynomolgus monkey antigens, gp130 (human—SEQ ID NO: 45, cynomolgus—SEQ ID No: 46) and IL27RA (human—SEQ ID NO: 41, cynomolgus—SEQ ID No: 42), was measured by surface plasmon resonance by anti-FAB capture of mAb-4894 (containing arms of anti-IL27RA-4880 EE and anti-gp130-4875 RR) on a biacore chip. Human or cynomolgus monkey IL27RA or gp130 was flowed over the chip and on and off rates were determined, and the affinity constants calculated.

As shown in Table 6, mAb-4894 bispecific antibody is able to bind to both human and cynomolgus gp130 and IL27RA. The binding affinities of the two arms of mAb-4894 are tuned to have ˜1000-fold difference with close to 0.1 nM affinity to the IL27RA subunit, but more than 100 nM affinity for the broadly distributed gp130 subunit. This differential affinity is expected to allow sufficient potency for agonist activity while minimizing binding to other gp130-containing receptors.

TABLE 6
Biacore Affinities of mAb-4894 bispecific antibodies to human and cyno GP130 and
IL27R

| Analyte | Ligand | ka (1/Ms) | kd (1/s) | KD (nM) + Std | n |
|---|---|---|---|---|---|
| Human GP130 | mAb-4894 | 3.37E+05 | 5.66E-02 | 169.86 ± 27.67 | 2 |
| Cyno GP130 | mAb-4894 | 2.15E+05 | 4.03E-02 | 187.59 ± 1.42 | 2 |
| Human IL27R | mAb-4894 | 9.98E+05 | 1.10E-04 | 0.11 ± 0.01 | 2 |
| Cyno IL27R | mAb-4894 | 1.17E+06 | 1.95E-03 | 1.68 ± 0 | 2 |

### Materials and Methods

### Preparation of Biosensor Sensor Chip

An anti-Fab sensor chip was prepared by amine coupling of anti-human Fab antibody to all eight channels of CM5 sensor chip per manufacturer's instruction. The flow channels were activated by injecting a 1:1 mixture of 400 mM 1-Ethyl-3-(3 dimethylaminopropyl) carbodiimide hydrochloride (EDC) and 100 mM N hydroxysuccinimide (NHS) for 7 minutes at a flow rate of 10 μL/minute. Anti-Fab IgG antibody was diluted to 25 μg/mL in 10 mM sodium acetate pH 5.0 and injected over all flow cells for 7 minutes at 10 μL/minute. All channels were blocked with 1M Ethanolamine-HCl (ETH) for 7 minutes at 10 μL/minute. Final immobilization level of the capture antibody was approximately 14,000 resonance units (RU). The running buffer for immobilization and kinetics was HBS-EP+ (10 mM 4-(2-hydroxyethyl)-1-piperazineethanesulfonic acid (HEPES) pH 7.4, 150 mM sodium chloride, 3 mM ethylenediaminetetraacetic acid (EDTA), 0.05% (v/v) Tween-20).

### SPR Analysis

The binding affinities of mAb-4894 to human and cynomolgus gp130 and IL27RA were determined using a BIAcore 8K+ instrument (Cytiva) at 37° C. with a collection rate of 10 Hz. mAb-4894 bispecific antibody was diluted to 0.08 μg/mL in HBS-EP+ buffer and captured by the anti-Fab IgG immobilized on flow cell 2 of all eight channels for 100 seconds at a flow rate of 10 μL/minute to achieve a capture level of about 50 RU. Flow cell 1 for each channel was used as a reference flow cell. After antibody capture, three-fold dilution series of cytokines with concentrations ranging from 405 nM to 15 nM for huGP130 and cyGP130 and 45 nM to 1.67 nM for huIL27R and cyIL27R or HBS-EP+ buffer were injected over the sensor surface for 60 seconds at 50 μl/minute. The dissociation was monitored for 600 seconds and the surface was regenerated with 2 injections of 10 mM Glycine pH 1.7 at 30 μl/minute. The data was double referenced (Myszka, D., J. Mol. Recognit 1999; 279-284). Binding affinities and rate constants were determined for human and cyno GP130 and IL27R by fitting the resulting sensorgram data to a 1:1 Langmuir model in BIAcore Insight Evaluation software version 3.0.12.15655 (Cytiva).

### Example 7. Epitope Overlap Assessment of IL27RA Arm Against Ligand IL27

### Results

As both the IL27 ligand complex (composed of p28 and Ebi3) and mAb-4894 bind IL-27 receptor, it is possible that the ligand and mAb-4894 could bind to similar epitope(s) on the receptor complex, which thus will potentially result in antagonist activity of the IL27RA arm of mAb-4894 against the ligand. Therefore, the inventors evaluated potential epitope competition between binding domains of mAb-4894 and the ligand complex using the Octet system.

Briefly, two sets of sensor tips were first coated with IL27 recombinant ligand complex (complex generation described in Example. 1), then one set was allowed to be bound by saturation levels of IL27RA, before being subjected to the IL27RA binding clone 2255 (parental clone) to see if the ligand can bind to both IL27RA and antibody 2255 simultaneously. The other set, however, was incubated in plain buffer before being subjected to 2255 as a positive control for binding of 2255 with the ligand.

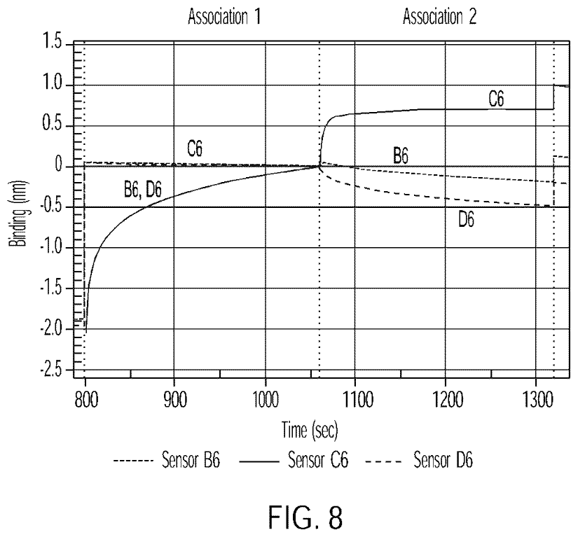

As shown in FIG. 8, 2255 failed to show any detectable binding to ligand sensor tip that has been bound by IL27RA (Sensor B6), while binding to naked sensor tip that was pre-exposed to only buffer (Sensor C6) was readily evident. This result implicates the potential antagonist activity of the IL27RA binding domain of mAb-4894 (Anti-IL27RA-4880 EE) against IL27 ligand when used as a monospecific antibody.

### Materials and Methods

Samples were diluted using 1× Sample Dilution Buffer (Sartorius) and it was also used as the assay buffer. The plate was shaken in between the experiment steps at 30° C. Sensors were used in duplicate.

Amine Reactive 2nd Generation (AR2G) sensors (Sartorius) were activated by dipping into a 1:1 mixture of 11.0 mM 1-Ethyl-3-(3 dimethylaminopropyl) carbodiimide hydrochloride (EDC) and 5.8 mM N hydroxysuccinimide (NHS) for 180 seconds. The sensors were then dipped into hIL27R-CH23Fc-Flag diluted to 25 μg/mL in 10 mM sodium acetate pH 5.0 for 300 seconds to direct immobilize hIL27R-CH23Fc-Flag onto the sensors. Lastly, the sensors were dipped into 1M Ethanolamine-HCl (ETH) for 200 seconds to block all amine binding sites. Final immobilization level of hIL27R was approximately 1.0 nm.

After dipping the same set of sensors into sample buffer for 100 seconds to establish a baseline, the sensors were dipped into hIL27:EBi3 at 300 nM for 260 seconds to allow the binding of hIL27:EBi3 to hIL27RA (Association 1). With a high concentration of hIL27:EBi3 at 300 nM, it is assumed that nearly all hIL27R on the sensors are bound to hIL27:EBi3. The sensors were immediately dipped into 300 nM GBT-IL27R-2255 for 260 seconds to assess whether GBT-IL27R-2255 can still bind the complex of hIL27:EBi3 and hIL27R-CH23Fc-Flag. A set of sensors were used as negative controls by dipping into buffer solution instead of GBT-IL27R-2255 after binding hIL27:EBi3 to show the dissociation of hIL27:EBi3 from immobilized hIL27RA. No binding should be observed with these sensors.

A set of sensors were used as positive controls by dipping into buffer solution, instead of hIL27:EBi3, after the baseline was established. These set of biosensors, without bound hIL27:EBi3, were then dipped into GBT-IL27R-2255 to demonstrate binding of GBT-IL27R-2255 to the hIL27RA immobilized on the sensors.

TABLE 7
sensor tip assignment for the epitope test

| Sensor | Direct Immobilization | 1^{st} Association | 2^{nd} Association |
|---|---|---|---|
| B6 | I127R | IL27: Ebi3 | GBT-IL-27R-2255 |
| C6 | I127R | Buffer | GBT-IL-27R-2255 |
| D6 | I127R | IL27: Ebi3 | Buffer |

### Example 8. Bioactivity Assessment of IL27RA/gp140 Bispecific on Human and Cynomolgus T Cells, Monocytes and Colonocytes

### Results

Key pharmacology endpoints were used to assess the bioactivity of the IL27RA/gp140 bispecific mAb-4894, specifically determining its ability to activate pathways and include expression of factors associated with agonism of Il27R, including assessing the phosphorylation of signal transducer and activator of transcription 1 and 3 (pSTAT1 and pSTAT3) in CD3+ T cells and activity of anti-inflammatory mediators such as programmed death ligand 1 (PD-L1) and the enzyme indoleamine-pyrrole 2,3-dioxygenase (IDO1) in CD14+ monocytes and primary cultured colonocytes. The results are described below and summarized in Table 9.

mAb-4894 induced the pSTAT1 and pSTAT3 in CD3+ T cells from human whole blood with the average EC50s of 0.43 nM and 0.23 nM, respectively (n=3). The window of STAT3 phosphorylation is smaller than STAT1 and showed higher donor-to-donor variability. In cynomolgus monkey whole blood, mAb-4894 induced the pSTAT1 and pSTAT3 in CD3+ T cells with average EC50s of 1.74 nM and 1.17 nM, respectively (n=4).

IDO1 is a cytosolic enzyme with a heme (Fe2+) prosthetic group that catalyzes tryptophan (Trp) catabolism and converts it to kynurenine (Kyn). The IDO1 pathway was originally described as an innate immune mechanism that defended the host organism against infections. Elevated levels of IDO1 strongly inhibit the proliferation and induces apoptosis of effector T cells and the accumulating Trp metabolites induces the differentiation of Tregs collectively giving rise to immunosuppression. The immunoprotective and immunosuppressive roles of IDO1 and Trp metabolites are tightly controlled by the stoichiometry of available local factors. The resultant effect of these local activities modulates IDO1 expression and helps maintain global immune homeostasis and peripheral immune tolerance. IL-27 is one of the immune modulatory cytokines which induces IDO1. In the human peripheral blood mononuclear cell (PBMCs) population, IL-27 bispecific antibody, mAb-4894, upregulated 001 expression in the CD14+ monocyte cytoplasm in a dose-dependent manner. The monocyte population (flow cytometric analysis) average EC50 is 0.005 nM (n=3). In primary cultured human colonocytes from two donors, IDO1 mRNA expression was significantly up-regulated by mAb-4894 with average EC50 of 20 nM. The activity of secreted IDO1 in cell culture supernatant was also evaluated by LC-MS assay. The IDO-1 activity assay for colonocytes average EC50 from the two donors was 5.4 nM (n=2 results are summarized in Table 9.

PD-L1 (CD274) is the dominant inhibitory ligand of PD-1 (Programmed cell death protein 1, also called CD279). Engagement of PD-1 by PD-L1 alters the activity of T cells in many ways, such as inhibiting T cell proliferation, survival, cytokine production, and other effector functions. In human whole blood, mAb-4894 induced PD-L1 expression in a dose dependent manner with average EC50 of 0.002 nM (n=2) results are summarized in Table 9.

TABLE 9
IL27RA/gp140 bispecific induced pSTAT1 and pSTAT3 and expression of PD-L1 and
IDO1 in Human and Cynomolgus T Cells, Monocytes and Colonocytes

| Assay | Species | Agonist | EC50(nM) | n |
|---|---|---|---|---|
| T Cell pSTAT1 | Human | mAb-4894 | 0.4 | 3 |
| | | | | 15 |
| | Cyno | mAb-4894 | 1.74 | 4 |
| T Cell pSTAT3 | Human | mAb-4894 | 0.2* | 3 |
| | Cyno | mAb-4894 | 1.17 | 4 |
| Monocyte PD-L1 | Human | mAb-4894 | 0.002 | 2 |
| Monocyte IDO1 | Human | mAb-4894 | 0.005 | 3 |
| Primary Colonocytes IDO1 | Human | mAb-4894 | 5.4 | 2 |
| | | | | 20 |

*Data was calculated by percentage of MAX (% MAX) induction due to high donor variability

### Materials and Methods

Whole blood from healthy human donors or untreated cynomolgus monkeys was collected in tubes containing anticoagulant, dispensed into 96-well plates, and warmed to 37° C. and stimulated by serial diluted concentrations of mAb-4894 or isotype control IgG8.8 antibody for 15 minutes(human) or 20 minutes(cynomolgus), followed by additional Lyse/Fix buffer (BD Biosciences, Cat #558049) according to the manufacturer's instructions. Cells were washed in FACS buffer and then permeabilized in pre-chilled 90% Methanol (for human samples) or in BD Phosflow Perm Buffer III (for cynomolgus samples). T cells were labeled with anti-CD3 antibody during agonist stimulation. Cells were washed again and incubated with fluorescently labeled antibodies recognizing phosphorylated STAT1(pY701) or STAT3 (pY705) (Table 10), followed by evaluation with flow cytometers and analysis with FlowJo software. Average relative fluorescence units (RFU) in the gated T cell population were calculated by multiplying the percentage of pSTAT+ cells by the mean fluorescence intensity (MFI). EC50 values (Table 9) were determined by graphing the concentration of agonist versus the RFU response from all donors using non-linear 3-parameter best-fit analysis software from GraphPad Prism 9 (GraphPad Software, Inc).

95 μl of healthy human whole blood was aliquoted into a deep 96-well plate, followed by treatment with 5 μl of serial diluted mAb-4894 or isotype control IgG8.8 antibody at 37° C. incubator for 3 hours. Cells were stained in the 37° C. incubator for extra 30 minutes with human FC block (BD Biosciences, Cat #564220), anti-CD3, anti-CD14, and anti-PDL-1 antibodies with the dilution factors of 1:10, 1:50, 1:5, and 1:20 respectfully (Table 10). Samples were lysed/fixed by pre-warmed 1XLyse/Fix buffer according to the manufacturer's instructions at 37 C for 20 mins. Cells were centrifuged, washed, and resuspended with FACS buffer. Monocyte PD-L1 expression was assessed by flow cytometers and MFI was analyzed with FlowJo software 10.7.1 (FlowJo, LLC). EC50 value (0.002 nM, Table 9) was determined by graphing the concentration of agonist versus MFI from all donors using non-linear 3-parameter best-fit analysis software from GraphPad Prism 9.

Human PBMCs was isolated from healthy human whole blood with density gradient centrifugation method, using SepMate tubes (Stemcell technologies, Cat #85450) and 15 ml of Ficoll-Paque premium reagent (GE Healthcare, Cat #17-5442-02). 190 μl of freshly isolated human PBMC cells (in 10% FBS RPMI at 5×10\hat{ }6 cells/mi) was aliquoted into a 96-well plate (Corning Costar, Cat #3879, Polypropylene), followed by treatment with 10 μl of serial diluted mAb-4894 or isotype control IgG8.8 antibody at 37° C. incubator for 20 hours. Cells were washed and further stained with Live/Dead Fixable Aquaous Cell staining kit (Invitrogen, Cat #L34966), human FC block, and anti-CD14 (Table 10), followed by fixation/permeabilization with Cyto Fix/Cyto Perm buffer (BD, Cat #554722) at 4° C. for 20 minutes, eventually stained with anti-IDO1 at 1:100 (Table 9) at 4° C. for 30 minutes. Monocyte IDO1 expression was assessed by flow cytometers and MFI was analyzed with FlowJo software 10.7.1. EC50 value (0.005 nM, Table 9) was determined by graphing the concentration of agonist versus MFI from all donors using non-linear 3-parameter best-fit analysis software from GraphPad Prism 9.

Two donors (Lot #02130, Lot #041417ABC) of human primary colonocytes (Cell Biologics, H-6047) were purchased and seeded in 48 well plates in complete DMEM/F-12 culture media until 90% confluency, followed by treatment with serial diluted mAb-4894 or isotype control IgG8.8 antibody at 37° C. incubator for 24 hours. Supernatant samples were saved and QCed, followed by protein precipitation with additional acetonitrile reagent. Isotope labeled Tryptophan and Kynurenine were added as internal standards. Supernatants were evaporated under nitrogen, reconstituted with injection buffer, and the production of kynurenine (Kyn) was assessed by LC-MS assay to reflect IDO1 activity. Average of EC50 value (5.4 nM, Table 9) was determined by graphing the concentration of agonist versus production of Kyn from all donors using non-linear 3-parameter best-fit analysis software from GraphPad Prism 9.

TABLE 10
antibodies used in biological assessment of IL27RA/gp130 bispecific

| | | Catalogue | Conjugated | Clone |
|---|---|---|---|---|
| Antibody Name | Vendor | Number | Fluorophore | Number |
| Mouse Anti-Human CD3 | BD Biosciences | 557705 | Alexa Fluor ® | SP34-2 |
| | | | 488 | |
| Mouse Anti-Human CD3 | BD Biosciences | 562426 | BV421 | UCHT1 |
| Mouse Anti-Human CD4 | BD Biosciences | 555346 | FITC | RPA-T4 |
| Mouse Anti-Human CD14 | BD Biosciences | 555398 | PE | M5E2 |
| Mouse Anti-Human CD25 | BD Biosciences | 341009 | PE | 2A3 |
| Mouse Anti-Human CD127 | BD Biosciences | 560822 | PE-Cy7 | HIL-7R- |
| | | | | M21 |
| Mouse Anti-Human | BD Biosciences | 563741 | APC | MIH1 |
| CD274 (PD-L1) | | | | |
| Mouse Anti-Human FOXP3 | BD Biosciences | 560045 | Alexa Fluor ® | 259D/C7 |
| | | | 647 | |
| Mouse Anti-Human IDO1 | BD Biosciences | 566648 | Alexa Fluor ® | V50-1886 |
| | | | 648 | |
| Mouse Anti-Human LAG3 | BD Biosciences | 745640 | BUV395 | T47-530 |
| Mouse Anti-Human TIM-3 | BD Biosciences | 565566 | BV711 | 7D3 |
| Mouse Anti-Stat1 (pY701) | BD Biosciences | 612597 | Alexa Fluor ® | 4a |
| | | | 647 | |
| Mouse Anti-Stat3 (pY705) | BD Biosciences | 557815 | Alexa Fluor ® | 4/P- |
| | | | 647 | STAT3 |
| Human IL-4 Antibody | Invitrogen | 16-7048-81 | None | MP4- |
| | | | (Functional | 25D2 |
| | | | Grade) | |

### Example 9 Biological Effects of IL27RA/Gp140 Bispecific on T Helper and T Reg Cells

### Results

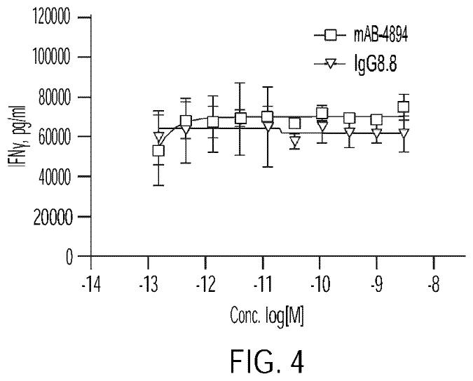

CD4+ T-helper cells play a variety of important roles in the development and maintenance of various autoimmune diseases including IBD. Based upon the differentially secreted cytokine panels which in turn mediate distinctive cellular activities, CD4+ T helper cells can be characterized into distinct subtypes: Th1, Th2, and Th17 cells, etc. In vitro, naïve CD4+ T cells can be induced and differentiated into these three types of T helper cells. During the skewing period, mAb-4894 upregulated IFNγ and T-bet expression during Th1 cell differentiation (data not shown) but had no effect on the expression of the pro inflammatory IFNγ in fully differentiated Th1 cells suggesting that mAb-4894 does not induce a pro-inflammatory response, as shown in FIG. 4. mAb-4894 down-regulated GATA-3 and IL-13 expression during Th2 cell differentiation (data not shown) and down-regulated IL-17A expression during Th17 cell differentiation. mAb-4894 also down-regulated IL-17A and Granulocyte Macrophage Colony Stimulating Factor (GM-CSF) in fully differentiated Th17 cells. Data is shown in Table 11.

TABLE 11
Down regulation of IL-17A in Th17 cells

| Assay | Species | Agonist | IC50(nM) | n | Assay |
|---|---|---|---|---|---|
| Th17 Cells IL- | Human | mAb-4894 | 0.0055 | 2 | Th17 Cells IL- |
| 17A | | | | | 17A |

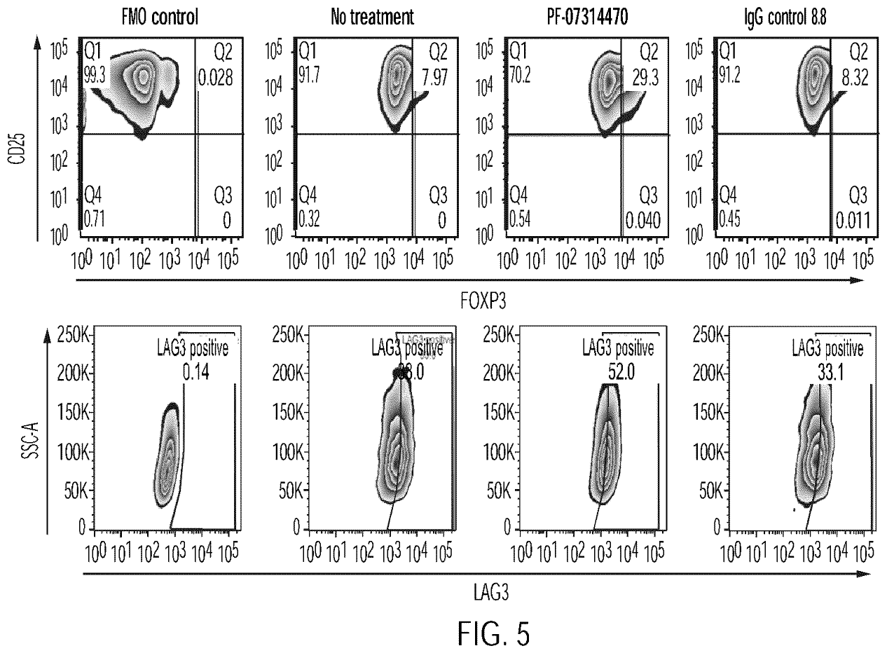

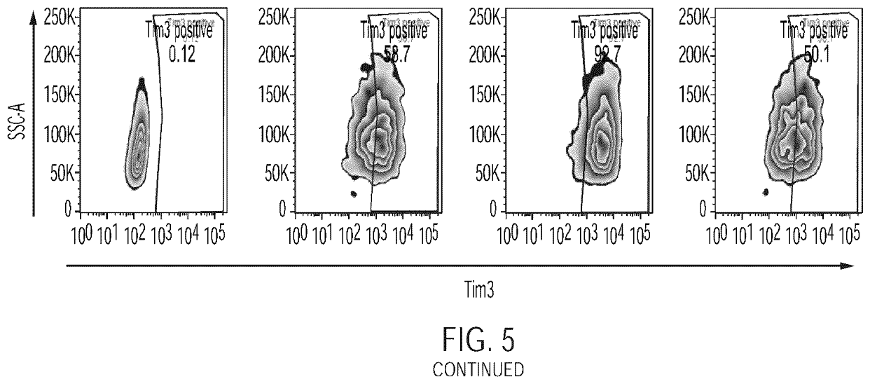

Regulatory T (Treg) cells are essential for maintaining peripheral tolerance, preventing autoimmune diseases, and limiting chronic inflammatory diseases. There are two types of Tregs: natural Tregs (nTreg) and inducible Tregs (iTreg). Tregs also express several immune checkpoint molecules including Tim-3 and LAG-3 which deliver negative immune modulatory signals on engagement with T cell activation. mAb-4894 induced more CD4+CD25+FOXP3+ iTreg from naïve CD4+ T cells compared to negative control antibody 8.8, upregulated the LAG3+ population, upregulated the Tim-3 expression level, and increased the Tim-3+ cell population as well. In nTregs, mAb-4894 upregulated IL-10 gene expression in 2 donors and upregulated LAG3 expression at both transcription level (n=2) and protein level (n=2). Data are shown in FIG. 5.

mAb-4894 was tested in three types of dendritic cells (DCs) differentiation conditions: immature DCs, immunogenic DCs and tolerogenic DCs. CD83 expression in the periphery may have functional significance and influence on lymphocyte maturation, survival, or function. The overexpression of CD83 by antigen presenting cells (APCs) is reported to enhance T cell proliferation in vitro. Immunoglobulin-like transcript 4 (ILT4) is an immunosuppressive molecule predominantly expressed in myeloid cells. mAb-4894 significantly down-regulated cell surface CD83 expression and up-regulated ILT4 expression, which may also contribute to mAb-4894's induced inhibitory effect on the allogenic T cell proliferation mediated by all three types of DCs. Other markers such as HLA-DR and PD-L1 were also up-regulated by bispecific antibody mAb-4894 in immature DCs and immunogenic DCs in two tested donors.

### Materials and Methods

Naive human CD4+ T cells were isolated from human Leukopak with Easysep Human Naïve CD4+ T cell Isolation kit (Stemcell technologies, Cat #17555). Cells were cultured in human Th17 differentiation cocktail (IL-6, TGFb1, IL-1b, IL-21 and IL-23, Table 12) and immunocult (Stemcell Technologies, Cat #10971) with or without serial diluted mAb-4894 at 37° C. incubator for 4 days. The concentration of IL-17A in the supernatant was evaluated by MSD assay (V-PLEX human IL-17A kit, Meso Scale Discovery, Cat #: K151RFD-2), and analyzed with GraphPad Prism 9. The IL-17A inhibition IC50 (0.0055 nM, Table 11) was averaged from two independent donors.

Naïve human CD4+ T cells were isolated from healthy human whole blood and treated with Th1 skewing cocktail (immunocult, IL-12, IL-18, anti-IL-4 antibody, Table 10) at 37° C. incubator for 7 days. After resting for 1 day, cells were treated with serial diluted mAb-4894 or isotype control IgG8.8 antibody (0.15 pM to 3 nM) for 1 day. Supernatant was saved and the production of IFNg was measured by MSD study (Meso Scale Discovery, Cat #K151QOD-2). Data was analyzed with Graphpad Prism software.

Naive human CD4+ T cells were isolated from human Leukopak with Easysep Human Naïve CD4+ T cell Isolation kit and cultured with RPMI complete culture medium, plus anti-CD3/CD28 beads (Dynabeads Human T-Activator CD3/CD28, Gibco, Cat #11131D), IL-2 (5 ng/ml) (Table 12), with or without mAb-4894 or isotype control IgG8.8 antibody treatment (1.35 nM) at 37° C. incubator for 4 days. After beads were removed, cells were rested in complete RPMI culture medium overnight and followed by fluorescently labeled antibodies recognizing CD4, CD25, LAG3, Tim3, CD127 and FOXP3 (Table 10). Data was assessed by flow cytometers and analyzed with FlowJo software 10.7.1.

TABLE 12
recombinant proteins used in biological assessment of II27RA/gp130
bispecific

| | | Catalogue |
|---|---|---|
| Name | Vendor | Number |
| Recombinant Human IL-1 | R&D | 201-LB |
| beta/IL-1F2 Protein | Systems | |
| Recombinant Human IL-2 | R&D | 202-IL |
| Protein | Systems | |
| Recombinant Human IL-6 | R&D | 206-IL |
| | Systems | |
| Recombinant Human IL-12 | R&D | 219-IL |
| Protein | Systems | |
| Recombinant Human IL- | R&D | |
| 18/IL-1F4 Protein | Systems | 9124-IL |
| Recombinant Human IL-21 | R&D | 8879-IL |
| Protein | Systems | |
| Recombinant Human IL-23 | R&D | 1290-IL |
| Protein | Systems | |
| Recombinant Human TGF- | | |
| beta 1 (Human Cell- | R&D | |
| expressed) Protein | Systems | 7754-BH |

### Example 9 In Vivo Biological Effects of IL27RA/Gp140 Bispecific

### Results

Changes in in vivo markers of pharmacological activity (IDO-1 activity, CXCL10 & CXCL11 concentration, platelet count) of the mAb-4894 anti-IL27RA/gp130 bispecific were demonstrated in cynomolgus monkeys. mAb-4894-related increases in serum chemokine concentrations were noted for CXCL10 (also known as IP-10) (range of 5.30×-20.96× compared to baseline) and CXCL11 (4.76×-74.44×). There were also observed increases in IDO-1 activity, measured as a function of unlabeled kynurenine and 13C-kynurenine. The greatest increases (4.29×-9.81× compared to baseline) were observed at 1 mg/kg/dose. The results are shown in Tables 17 and 18.

These results demonstrate that the anti-IL27RA/gp130 bispecific antibody mAb-4894 modulates pharmacological biomarkers in vivo that are consistent with IL-27 activity/agonism.

TABLE 17
Intravenous Dosing in Monkeys with mAb-4894 on Days 1 and 8 (Study 1)

| | | | Fold-Change Increase Compared to Pretreatment | | | |
|---|---|---|---|---|---|---|
| | | | Baseline | | | |
| Measurement | | | 0.1 mg/kg | 1 mg/kg | 10 mg/kg | 100 mg/kg |
| Assay | Parameter | Sex | (IV) | (IV) | (IV) | (IV) |
| IDO-1 | ¹³C-Labeled | M | 2.53 | 4.29 | 3.15 | 1.27 |
| Activity¹ | Kynurenine | F | 1.80 | 9.81 | 4.13 | 0.79 |
| | Unlabeled | M | 4.68 | 6.68 | 2.61 | 3.62 |
| | Kynurenine | F | 3.20 | 5.55 | 4.68 | 2.34 |
| Serum | CXCL10 | M | 14.08 | 13.70 | 16.31 | 20.37 |
| | | F | 5.30 | 10.87 | 10.18 | 20.96 |
| Chemokine | CXCL11 | M | 19.83 | 74.44 | 30.86 | 22.07 |
| Concentration² | | F | 4.76 | 9.71 | 28.69 | 10.22 |
| Plasma Exposure | | | | | | |
| AUC_{0-72 h} (ug * h/mL)³ | | M | 65 | 944 | 9890 | 125000 |
| | | F | 109 | 1260 | 12400 | 122000 |

¹Peak fold-change increase of IDO-1 activity measured in plasma on Days 1-15.
²Peak fold-change increase in the concentration of CXCL10 or CXCL11 protein in serum on Days 1-15.
³AUC of PF-07314477 concentration in plasma on Days 8-11.

TABLE 18
Intravenous or Subcutaneous Dosing in Monkeys with mAb-4894 on Days 1, 8, 15, 22
and 29 (Study 2)

| | | | Fold-Change Increase Compared to Pretreatment | | | | |
|---|---|---|---|---|---|---|---|
| | | | Baseline | | | | |
| Measurement | | | Vehicle | 0.01 mg/kg | 0.1 mg/kg | 1 mg/kg | 1 mg/kg |
| Assay | Parameter | Sex | (IV/SC) | (IV) | (IV) | (IV) | (SC) |
| IDO-1 | ¹³C- | M | 1.78 | 1.47 | 5.58 | 9.60 | 6.04 |
| Activity¹ | Labeled | F | 2.89 | 7.02 | 4.20 | 2.96 | 3.63 |
| | Kynurenine | | | | | | |
| | Unlabeled | M | 1.62 | 2.21 | 5.20 | 5.58 | 4.15 |
| | Kynurenine | F | 0.98 | 2.48 | 3.28 | 3.85 | 2.81 |
| Serum CXCL10 | | M | 1.57 | 2.04 | 18.76 | 9.63 | 15.82 |
| Concentration² | | F | 1.59 | 2.35 | 4.61 | 17.99 | 12.98 |
| Plasma Exposure | | | | | | | |
| AUC_{0-360 h} (ug * h/mL)³ | | M | NT | N/A | 31.7 | 632 | 352 |
| | | F | NT | N/A | 36.6 | 675 | 741 |

¹Peak fold-change increase of IDO-1 activity measured in plasma on Days 1-36.
²Peak fold-change increase in the concentration of CXCL10 protein in serum on Days 1-36.
³AUC of PF-07314477 concentration in plasma on Days 29-43.

### Methods

In Vivo Pharmacology of mAb-4894 in Cynomolgus Monkeys

Male and female cynomolgus monkeys of Mauritius origin, aged greater than 2.5 years were acclimated for a minimum of 30 days prior to initiating dosing. In study 1, dose groups containing one male and one female monkey were administered PF-08314470 at 0.1, 1, 10 or 100 mg/kg by intravenous (IV) injection on days 1 and 8. In study 2, dose groups containing one male and one female monkey were administered vehicle control by IV and subcutaneous (SC) injection, or PF-08314470 at 0.01, 0.1, or 1 mg/kg IV, or 1 mg/kg SC on days 1, 8, 15, 22 and 29. Blood was collected at various times over the course of each study for evaluation of indoleamine 2,3-dioxygenase 1 (IDO-1) enzymatic activity or serum chemokine concentration. Following the final dose, blood was collected at various time points over 72 hours (study 1) or 360 hours (study 2) for toxicokinetic analysis. Total mAb-4894 concentrations in serum were determined using a ligand-binding assay and the area under the curve (AUC) concentrations were assessed for each individual animal.

### IDO1 Activity Assay

Prior to dosing and at various time points after administration of mAb-4894, whole blood samples collected in anticoagulant were mixed ex vivo with 130-labeled tryptophan and stored at 37° C. After overnight incubation, plasma was separated by centrifugation and stored at −80° C. At the end of each study, the concentrations of 130-labeled and unlabeled kyenurenine (the end-products of IDO-1 enzymatic conversion of 130-labeled and endogenous tryptophan, respectively) in each plasma sample were measured by LC-MS/MS. The fold-change in both kynurenine products in each animal were determined by comparison to their respective concentration in pre-dose samples. For each animal the peak fold-change increase between initiation of dosing and seven days after the last dose were determined for each kynurenine product.

### Serum CXCL10 and CXCL11

Serum was collected at various time points from all animals and the concentration of the C—X—C chemokines, CXCL10 and CXCL11, were determined using ligand binding assays. The peak fold-change increase compared to pre-dose levels for each chemokine was determined in each animal.

### Example 11: Epitope of IL27RA/gp130 Bispecific

### Experimental Methods.

### IL27Ra (D1-D2)+FAb-2255.

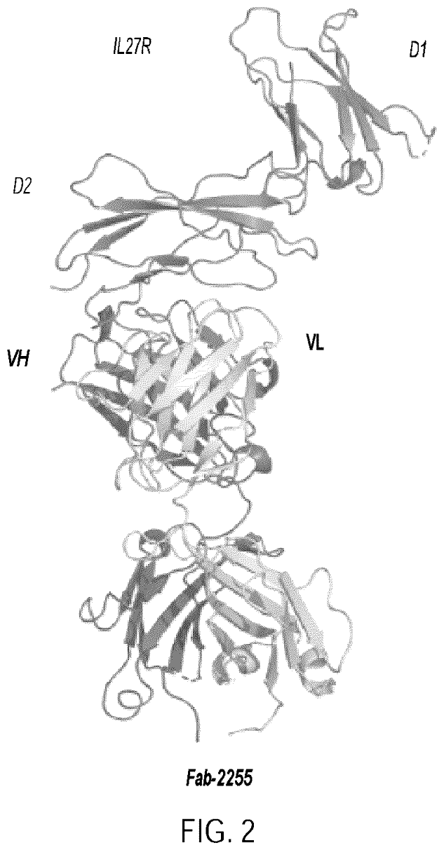

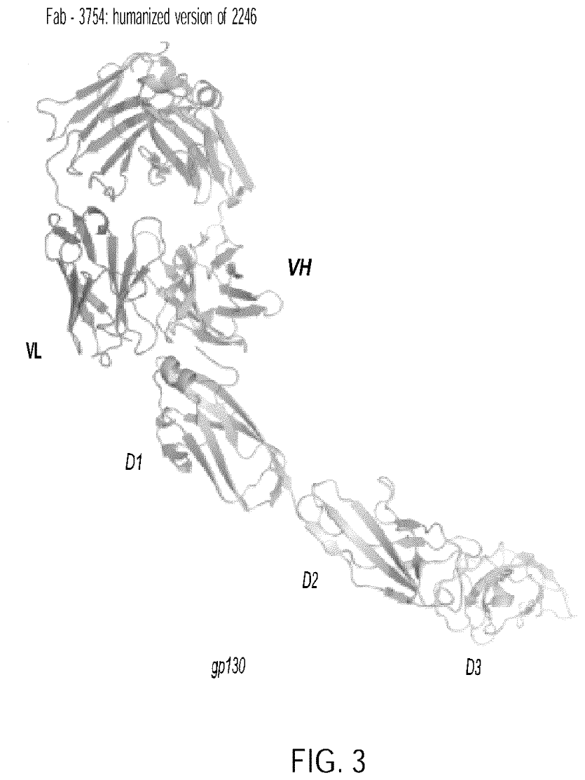

The co-crystals of IL27Ra(D1-D2) bound to the FAb fragment of GBT-IL27R-2255 were obtained by hanging-drop vapor-diffusion method from a condition containing 20% PEG 4000, 200 mM Lithium sulfate, 100 mM MES pH 6. The crystals had symmetry consistent with monoclinic space group P21 with unit cell parameters a=83.97 Å; b=235.16 Å; c=84.22 Å, beta=90.6° and with 4 copies of complexes in the crystallographic asymmetric unit. The crystals were flash frozen in liquid nitrogen using 20% EG as a cryoprotectant solution. A data set to a 3.2 Å resolution was collected from a single frozen crystal at IMCA beamline 17-ID at the Argonne National Laboratory (APS). The data were processed and scaled using autoPROC, and the final dataset was 82.5% complete. The structure was solved by molecular replacement with PHASER. Several iterative rounds of manual adjustment and model rebuilding using COOT and crystallographic refinement using autoBUSTER yielded the final model with a crystallographic R_{work} of 23.1% and R_{free} of 26.2%, where R_{work}=∥F_{obs}|−|F_{calc}∥/|F_{obs}| and R_{free} is equivalent to R_{work}, but calculated for a randomly chosen 5% of reflections omitted from the refinement process. A ribbon diagram of the cocrystal structure is shown in FIG. 2.

gp130(D1-D2-D3)+Fab-3754 (Humanized 2246)

The co-crystals of gp130(D1-D2-D3) bound to Fab-3754 (a humanized variant of antibody 2246) were obtained by hanging-drop vapor-diffusion method from a condition containing 20% PEG 6000, 200 mM Calcium chloride, 100 mM MES pH 6. The crystals had symmetry consistent with primitive space group P1 with unit cell parameters a=67.98 Å; b=93.97 Å; c=100.63 Å, alpha=62.8°, beta=77.36°, gamma=86.1° and with 2 copies of complexes in the crystallographic asymmetric unit. The crystals were flash frozen in liquid nitrogen using 20% glycerol as a cryoprotectant solution. A data set to a 2.72 Å resolution was collected from a single frozen crystal at IMCA beamline 17-ID at the Argonne National Laboratory (APS). The data were processed and scaled using autoPROC, and the final dataset was 72% complete. The structure was solved by molecular replacement with PHASER. Several iterative rounds of manual adjustment and model rebuilding using COOT and crystallographic refinement using autoBUSTER yielded the final model with a crystallographic R_{work} of 22% and R_{free} of 24.4%, where R_{work}=∥F_{obs}|−|F_{calc}∥/|F_{obs}| and R_{free} is equivalent to R_{work}, but calculated for a randomly chosen 5% of reflections omitted from the refinement process. A ribbon diagram of the cocrystal structure is shown in FIG. 3.

### Results

The IL-27RA and gp130 amino acids in close contact (within 3.80A) of the anti-IL-27RA antibody 2255 and the gp130 antibody 3754 are shown in Tables 19 and 20, respectively. Briefly, the epitopes of the two Fabs are described as follows: Fab-2255 binds exclusively to domain D2 of the receptor utilizing all CDR loops for contact except CDR-L1. The binding interface is dominated by polar and electrostatic interactions (9 hydrogen-bonding contacts total) with the heavy chain loops CDR-H2 and -H1 contributing most to the interaction. The buried surface area on the antigen is extensive −1,714 Å².

Fab-3754 binds at the tip of domain D1 of gp130. All CDR loops are in contact with the antigen except CDR-L2. The binding interface is predominantly polar with 9 specific hydrogen-bonding contacts at its center. The heavy chain loops CDR-H3, -H2, -H1 contribute most to the interaction. The size of the binding interface is 1,461 Å² which is on the low-to-medium end of mAb-antigen interfaces.

TABLE 19
IL-27RA residues within 3.80 Angstroms of antibody 2255

| | | | Antibody |
|---|---|---|---|
| Antigen Chain | Antigen Residue | Antibody Chain | Residue |
| I | 100 | H | 57 |
| I | 102 | H | 57 |
| I | 103 | H | 56 |
| I | 104 | H | 52 |
| I | 104 | H | 53 |
| I | 104 | H | 54 |
| I | 104 | H | 55 |
| I | 104 | H | 56 |
| I | 104 | H | 57 |
| I | 105 | H | 53 |
| I | 106 | H | 31 |
| I | 106 | H | 33 |
| I | 106 | H | 50 |
| I | 106 | H | 53 |
| I | 107 | H | 31 |
| I | 107 | H | 32 |
| I | 109 | H | 2 |
| I | 109 | H | 32 |
| I | 109 | H | 98 |
| I | 110 | L | 46 |
| I | 110 | L | 49 |
| I | 114 | L | 49 |
| I | 118 | H | 52 |
| I | 118 | H | 57 |
| I | 118 | H | 59 |
| I | 119 | H | 57 |
| I | 120 | H | 57 |
| I | 155 | L | 93 |
| I | 158 | L | 94 |
| I | 160 | L | 92 |
| I | 197 | H | 31 |
| I | 198 | H | 31 |

TABLE 20
gp130 residues within 3.80 Angstroms of antibody 3754

| | | | Antibody |
|---|---|---|---|
| Antigen Chain | Antigen Residue | Antibody Chain | Residue |
| G | 35 | H | 100 |
| G | 34 | H | 101 |
| G | 35 | H | 101 |
| G | 37 | H | 101 |
| G | 35 | H | 102 |
| G | 36 | H | 102 |
| G | 37 | H | 102 |
| G | 1 | H | 31 |
| G | 2 | H | 31 |
| G | 35 | H | 32 |
| G | 35 | H | 33 |
| G | 36 | H | 33 |
| G | 91 | H | 33 |
| G | 2 | H | 52 |
| G | 91 | H | 52 |
| G | 90 | H | 56 |
| G | 87 | H | 58 |
| G | 88 | H | 58 |
| G | 89 | H | 58 |
| G | 35 | H | 98 |
| G | 37 | L | 33 |
| G | 86 | L | 93 |
| G | 86 | L | 94 |
| G | 87 | L | 94 |
| G | 87 | L | 95 |

### Example 12: Expression, Purification, and Characterization of Alternative Bispecific Format Variant

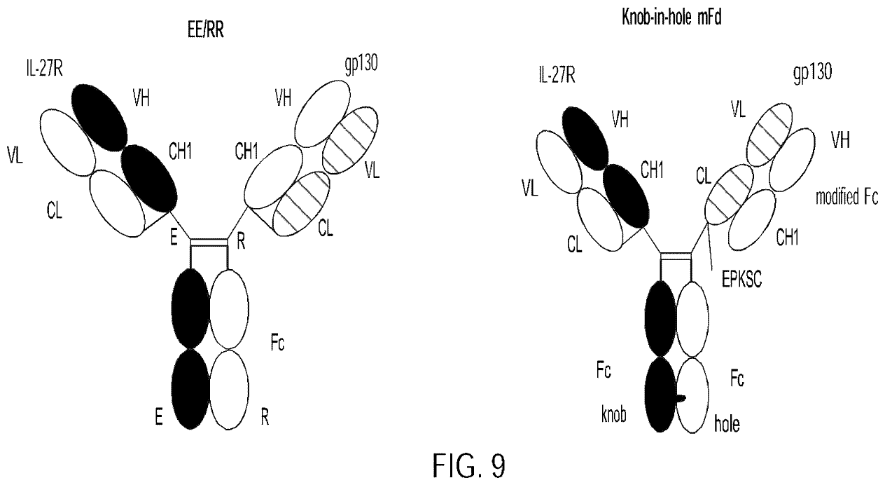

A IL27RA/gp130 bispecific antibody, GBT-IL-27R-4933, was constructed using identical variable heavy and variable light regions from GBT-IL-27R-4894 in an alternative format to examine the impact of modifying the heterodimerization strategy on bispecific function and manufacturing properties. The alternative format, utilizing knob-in-hole Fc (KiH) heterodimerization, is referred to as KiH mFd (FIG. 9). It utilizes a Fab arrangement referred to as “modified Fd” (mFd) and was employed for the anti-gp130 FAb. In this arrangement, the anti-gp130 light chain (Table 21) was joined to the lower hinge region (DKTHTCPPCP) of the human IgG1-effector function-minimized Fc region to generate the VL-CL-Fc protein chain of this bispecific modality. A modified Fd chain was designed to pair with the anti-gp130 LC within the VL-CL-Fc chain, and it is composed of an anti-gp130 VH, human IgG1-CH1 (Table 21) and the upper human IgG1 hinge amino acids EPKSC harboring Cys at linear position 221 for interchain disulfide formation with Cys (linear position 214) in the anti-gp130 Fab Kappa constant domain. The GBT-IL-27R-4933 bispecific was constructed using the KiH mFd-bispecific modality.

Specifically, the LC of gp130-4875 (SEQ ID NO: 24) was joined to the to the lower hinge region of an IgG1-effector function-minimized Fc engineered with the Hole heterodimerization mutations T(360)S, L(362)A and Y(401)V along with S(348)C (linear numbering) to generate the GBT-IL-27R-4933 VL-CL-Fc chain (Table 21). The GBT-IL-27R-4933 modified Fd chain (Table 21) was constructed by fusing gp130-4875 VH (SEQ ID NO: 21) to the IgG1 CH1 (SEQ ID NO: 9) and upper hinge sequence EPKSC. The IL-27RA 4880 VH (SEQ ID NO: 7) was fused to the IgG CH1 (SEQ ID NO: 9) and an IgG1-effector function-minimized Fc harboring the Knob heterodimerization mutation T(364)W plus Y(347)C (linear numbering) to generate the GBT-IL-27R-4933 HC (Table 21). The GBT-IL-27R-4933 LC is identical to the IL-27RA 4880 LC (SEQ ID NO: 14).

TABLE 21
Sequences of chains of GBT-IL-27R-4933

| Chain | Sequence |
|---|---|
| gp130 VL-CL- | DIQMTQSPSS LSASVGDRVT ITCRASQSVS SSYLAWYQQK PGKAPKLLIY |
| Fc-(hole) | GTESRATGVP SRESGSGSGT DETLTISSLO PEDFATYYCQ QYGGYPTEGG |
| SEQ ID NO: | GTKVEIKRTV AAPSVFIFPP SDEQLKSGTA SVVCLLNNFY PREAKVQWKV |
| 57 | DNALQSGNSQ ESVTEQDSKD STYSLSSTLT LSKADYEKHK VYACEVTHQG |
| | LSSPVTKSEN RGECDKTHTC PPCPAPEAAG APSVELFPPK PKDTLMISRT |
| | PEVTCVVVDV SHEDPEVKEN WYVDGVEVHN AKTKPREEQY NSTYRVVSVL |
| | TVLHQDWLNG KEYKCKVSNK ALPAPIEKTI SKAKGQPREP QVYTLPPCRE |
| | EMTKNQVSLS CAVKGFYPSD IAVEWESNGQ PENNYKTTPP VLDSDGSFFL |
| | VSKLTVDKSR WQQGNVESCS VMHEALHNHY TOKSLSLSPG K |
| gp130 VH- | QVQLVQSGAE VKKPGSSVKV SCKVSGGSIS SYYWSWVRQA PGQGLEWIGR |
| CH1 (mFd) | IYITGNTNYN PSLKDRVTMT VDTSTSTHYM ELSSLRSEDT AVYYCAREGP |
| SEQ ID NO: | KLGIFDYWGQ GTLVTVSSAS TKGPSVFPLA PSSKSTSGGT AALGCLVKDY |
| 58 | FPEPVTVSWN SGALTSGVHT FPAVLOSSGL YSLSSVVTVP SSSLGTQTYI |
| | CNVNHKPSNT KVDKKVEPKS C |
| IL-27R heavy | EVOLVESGGG LVQPGGSLRL SCAASGFTFE YEGLOWVRQA PGKGLEWIGY |
| chain (knob) | TSSGGNYEYY EETLKGRETI SRDNAKNSLY LOMNSLRAED TAVYYCARTK |
| SEQ ID NO: | TGDYWGQGTL VTVSSASTKG PSVFPLAPSS KSTSGGTAAL GCLVKDYFPE |
| 59 | PVTVSWNSGA LTSGVHTFPA VLQSSGLYSL SSVVTVPSSS LGTQTYICNV |
| | NHKPSNTKVD KKVEPKSCDK THTCPPCPAP EAAGAPSVEL FPPKPKDTLM |
| | ISRTPEVTCV VVDVSHEDPE VKENWYVDGV EVHNAKTKPR EEQYNSTYRV |
| | VSVLTVLHQD WINGKEYKCK VSNKALPAPI EKTISKAKGQ PREPQVCTLP |
| | PSREEMTKNQ VSLWCLVKGF YPSDIAVEWE SNGQPENNYK TTPPVLDSDG |
| | SFFLYSKLTV DKSRWQQGNV FSCSVMHEAL HNHYTQKSLS LSPGK |
| IL-27R light | RFSGSGSGTD FTLTISSLOP EDFATYYCLQ SKOYPYTFGQ GTKLEIKRTV |
| chain (part | AAPSVFIFPP SDEQLKSGTA SVVCLLNNFY PREAKVOWKV DNALQSGNSQ |
| identical to the | ESVTEQDSKD STYSLSSTLT LSKADYEKHK VYACEVTHQG LSSPVTKSEN |
| IL-27RA 4880 | RGEC |
| LC | |
| (SEQ ID NO: | |
| 14) | |

Knob or hole mutations are indicated in bold type. Cysteine residues involved in interchain disulfide formation between gp130 CH1 (mFd) and gp-130 Fab kappa constant domain in gp130 VL-CL-Fc-(hole) are shown as underlined italics. Lower hinge in gp130 VH-CH1 (mFd) and upper hinge in gp130 VL-CL-Fc-(hole) are indicated by italics.

The KiH-mFd IL27RA/gp130 bispecific antibody, GBT-IL-27R-4933, was generated by coexpression of all four protein chains either transiently in Expi293F™ host cells using the manufacturer's recommended protocol or by generation of a stable CHO cell line using methods described in Example 5. Purification of the bispecific was carried out in a multi-step process, first using standard Protein A (MabSelect SuRe LX) and TMAE chromatography (run at 50 mM Tris, pH8.3) as described in Example 5, followed by mixed-mode anion exchange chromatography (CaptoAdhere, Cytiva) and hydrophobic interaction chromatogaphy (HIC) on a Phenyl 650M column (TOSOH). Final buffer exchange was conducted as described in Example 5.

A comparison of process yield and purity of the two IL27RA/gp130 bispecific antibodies, GBT-IL-27R-4894 (EE-RR format) and GBT-IL-27R-4933 (KiH-mFd format), is shown in Table 22. Significant differences were observed between the two formats. While GBT-IL-27R-4894 (EE-RR format) required two independent Protein A chromatography columns for initial purification of the EE and RR parental molecules prior to redox, the overall process required fewer non-standard steps and produced protein with higher yield and purity than GBT-IL-27R-4933 (KiH-mFd format). Stable CHO pool expression of GBT-IL-27R-4933 was 0.35/g/L as measured by Protein A yield, while expression of GBT-IL-27R-4894 as ˜3-fold higher. Following Protein A purification, the EE and RR arms of GBT-IL-27R-4894 were >99% pure as measured by SEC, while GBT-IL-27R-4933 was only 55% pure, with close to 40% high molecular mass species, which was not removed by TMAE. While the high molecular mass material was removed from GBT-IL-27R-4933 by the mixed-mode and HIC steps, 5-10% residual homodimer remained in the final product. By contrast, the GBT-IL-27R-4894 (EE-RR format) protein could be purified to >99% purity, with <0.01% homodimer remaining in the final material.

TABLE 22
Purification process of GBT-IL-27R-4894 (EE-RR format) and GBT-IL-27R-4933 (KiH-
mFd format) expressed from stable CHO

| | GBT-IL-27R-4894 (EE-RR | GBT-IL-27R-4933 (KiH-mFd |
|---|---|---|
| Process step | format) | format) |
| Number of cell lines | 2 | 1 |
| Yield post Protein A | 0.9-1.3 g/L | 0.35 g/L |
| Purity post Protein A | >99% (residual HMMS) | 55% (residual HMMS, LMMS) |
| (aSEC) | | |
| Percent homodimer post | N/A | 5-10% |
| Protein A | | |
| TMAE recovery | 97% | ~80% |
| Additional column steps | HIC | Mixed-mode anion exchange, |
| | | HIC, additional may be required |
| Final product purity | >99% | 96% |
| (aSEC) | | |
| Homodimers in final | <0.01% | 5-10% |

product

N/A, not applicable;
SEC, size-exclusion chromatography;
TMAE, trimethylaminoethyl;
HMMS, high molecular mass species;
LMMS, low molecular mass species

### Biophysical and Bioanalytical Assessment:

The IL27RA/gp130 bispecific antibodies GBT-IL27R-4933 (knob-in-hole) and GBT-IL27R-4894 (EE-RR) were subjected to extensive characterization to evaluate bioanalytical and biophysical properties of these complex molecules. In particular, the following methods were used to assess key molecular properties: analytical size-exclusion chromatography (aSEC) to determine percent high molecular mass species (HMMS) as an indicator of aggregation, thermal stability using Differential Scanning calorimetry (DSC), non-reduced capillary gel electrophoresis (cGE) to assess percent peak of interest (P01), imaged capillary electrophoresis (iCE) to evaluate charge heterogeneity, dynamic light scattering (DLS) to evaluate viscosity, and AC-SINS, DNA ELISA, insulin ELISA, and human FcRn chromatography for non-specificity.

Analytical SEC was performed using a YMC-Pack Diol-200 SEC column in 20 mM Na₃PO₄, 400 mM NaCl, pH 7.2 buffer. Retention time (min) and peak width at 50% height (min) of the main peak as well as the area under the curve of the main peak (P01), low molecular mass species (LMMS) and HMMS peaks were recorded and used to calculate percent main peak (P01), HMMS and LMMS. Protein samples were concentrated to approximately 150 mg/mL and held at 25 C for up to 6 weeks, or concentrated to 5 mg and subjected to 40 C stress for up to 4 weeks.

For the DSC method, samples at 0.3 mg/mL were dispensed into the sample tray of a MicroCal PEAQ-DSC Automated (Malvern Panalytical, Ltd), equilibrated for 5 minutes at 10° C., and then scanned up to 110° C. at a rate of 100° C. per/hour. Raw data was baseline corrected and the protein concentration was normalized. MicroCal PEAQ-DSC software (Malvern Panalytical, Ltd.) was used to fit the data to a non-two-state model with an appropriate number of transitions.

Viscosity was measured by a DLS (dynamic light scattering) bead-based method at concentrations up to ˜175 mg/mL. Purified antibodies in phosphate buffered saline pH 7.2 (PBS) were extensively dialyzed against 20 mM histidine, 8.5% sucrose, 0.05 mg/mL EDTA pH 5.8 using membrane cassette devices 10K MWCO (Thermo Scientific). Antibodies were concentrated using Viva spin centrifugal concentrators 10K MWCO (GE Healthcare). Proteins were concentrated to ˜175 mg/mi and lower concentrations were prepared by diluting with sample buffer. All samples were prepared at 12 μL. 300 nm beads (Nanosphere, Thermo Scientific) were added to the protein samples and buffer blank. The beads were diluted 1:100 in 20 mM histidine, 8.5% sucrose, 0.05 mg/mL EDTA pH 5.8, and 0.75 μL diluted beads were spiked into the protein sample. The protein/bead and buffer/bead samples were mixed by gently vortexing. 8 μL samples were transferred to a 1536 well plate (SensoPlate, glass bottom, Greiner Bio-One) for analysis by DLS. The plate was sealed with optically clear tape and centrifuged at 2000 RPM for 2 minutes to remove bubbles. DLS measurements were made using a DynaPro Plate Reader (Wyatt Technology, Santa Barbara, Calif.). Samples were incubated at 25° C. and measured with 15 consecutive 25 second acquisitions. Radius of the bead was averaged for data acquisitions that had acceptable decay curves. The viscosity was calculated based on the Stokes-Einstein equation. Sample viscosity was calculated as the measured apparent radius divided by the nominal bead radius 10 times 0.893 cP, the viscosity of water at 25° C.

Non-reduced cGE was performed using the Caliper LabChip GXII (PerkinElmer Inc., Hopkinton, MA) according to manufacturer's recommended protocol. Protein Simple iCE3 instrument with PrinCE Autosampler (ProteinSimple, San Jose, CA) was used to analyze charge heterogeneity according to the following specifications. Proteins were diluted to 2 mg/mL in water. Sample diluent is composed of 0.01 mg/mL pI marker 7.55, 0.01 mg/mL pI marker 10.1, 1.0% Pharmalyte pH 5-8, 3.0% Pharmalyte pH 8-10.5, 0.25% methyl cellulose, 2.0 M urea, and 4.25 mM Arginine. Samples contained 15 μL protein at 2 mg/mL and 85 μL sample diluent. Samples were focused for 1 minute at 1500 Volts and then 9 minutes at 3000 Volts. The IL27RA/gp130 bispecific antibodies were additionally subjected to DNA/insulin polyreactivity, AC-SINS self-association, and human FcRn chromatography biophysical characterization assays to evaluate non-specificity properties as described in Example 4.

The summary of results for the biophysical and bioanalytical evaluation of GBT-IL27R-4933 (knob-in-hole) and GBT-IL27R-4894 (EE-RR) indicate that both generally have favorable molecular properties comparable to standard well-behaved antibodies (Table 23). Both exhibit good thermal stability with Tm1 values >65° C., and low viscosity (at or below 15 cP at 175 mg/ml) in 20 mM histidine, 8.5% sucrose, 0.05 mg/mL EDTA pH 5.8. Both variants present acceptable non-specific binding profiles and acceptable stability by aSEC, cGE, and iCE following challenge at 25 C and 40 C or incubation in the presence of mouse serum (Table 23).

TABLE 23
Summary of key biophysical and bioanalytical properties evaluated for bispecific
variants

| Property | GBT-IL-27R-4933 (KiH) | GBT-IL-27R-4894 (EE/RR) |
|---|---|---|
| DSC | TM1 - 70.81 ± 0.03 in His- | Tm1 - 70.70° C. in His-sucrose |
| | sucrose platform buffer | platform buffer |
| Viscosity (DLS) | ~13.2 cP at 175 mg/ml | ~15 cP at 175 mg/ml |
| FcRn chromatography | 0.01 min (rRT) | 0.02 min (rRT) |
| Non-specificity (AC-SINS | 0.1 | 1.0 |
| score) | | |
| Non-specificity (DNA ELISA | 3.3 | 4.4 |
| score) | | |
| Non-specificity (insulin ELISA | 3.2 | 4.4 |
| score) | | |
| Stability at ~150+ mg/ml in 20 | Acceptable HMMS/LMMS at 6 | Aceptable HMMS/LMMS at 6 |
| mM Histidine/8.5% sucrose | weeks | weeks |
| pH 5.8 | | |
| 25° C. Stability iCE (~150 | Minimal increase in | Minimal increase in |
| mg/ml) | acidic/basic species, 6 weeks | acidic/basic species, 6 weeks |
| | 25° C. (20 mM Tris/8.5% | 25° C. |
| | sucrose pH 7.5 sample | |
| | compromised) | |
| 25° C. Stability cGE (~150 | No significant fragmentation, 6 | No significant fragmentation, 6 |
| mg/ml) | weeks 25° C. | weeks 25° C. |
| 40° C. Stability aSEC (5 mg/ml) | Minimal increase in | Minimal increase in |
| | HMMS/LMMS, 4 weeks 40° C. | HMMS/LMMS, 4 weeks 40° C. |
| 40° C. Stability aSEC-MALS | (Assay not run) | Higher order oligomer not |
| (5 mg/ml) | | detected |
| 40° C. Stability iCE (5 mg/ml) | Acceptable rate of increase | Acceptable rate of increase |
| | for acidic/basic species, 4 | for acidic/basic species, 4 |
| | weeks 40° C. | weeks 40° C. |
| 40° C. Stability cGE (5 mg/ml) | Minimal fragmentation, 4 | Minimal fragmentation, 4 |
| | weeks 40° C. | weeks 40° C. |
| 40° C. Stability (5 mg/ml) | No significant loss of activity | Sight loss in functional |
| Bioassay | (binding ELISA/pSTAT1 | activity; binding to gp130 and |
| | assays) | IL27R retained by ELISA |
| Proteolytic Susceptibility | No significant fragmentation | No significant fragmentation |
| (mouse serum) | detected | detected |

### Functional Activity:

A comparison of the functional activity showed that GBT-IL27R-4933 (knob-in-hole) had slightly lower agonistic potency than that of GBT-IL27R-4894 (EE-RR) in three assays in human whole blood. (Table 24). Assays were performed as described in Example 8. The EC₅₀s for STAT1 phosphorylation in CD3+ T cells were 0.233 and 0.083 nM for −4933 and −4894, respectively (2.8-fold ratio). The EC₅₀s for PD-L1 upregulation in CD14+ monocytes were 0.006 and 0.002 nM for −4933 and −4894, respectively (3-fold ratio), and the EC₅₀s for IDO1 expression in CD14+ monocytes were 0.015 and 0.008 nM for −4933 and −4894, respectively (1.8-fold ratio).

Based on the observed differences in bioactivity and manufacturing properties, GBT-IL27R-4894 (EE-RR format) displayed a consistent advantage over the GBT-IL27R-4933 (knob-in-hole).

TABLE 24
Activity of IL27RA/gp130 bispecific antibodies in EE/RR and knob-in-hole formats in
whole blood

| | pSTAT1 in CD3+ T | PD-L1 in CD14+ | IDO1 in CD14+ |
|---|---|---|---|
| | cells EC₅₀ (nM) | monocytes EC₅₀ (nM) | monocytes EC₅₀ (nM) |
| GBT-IL27R-4894 | 0.083 | 0.002 | 0.008 |
| (EE/RR) | | | |
| GBT-IL27R-4933 | 0.233 | 0.006 | 0.015 |
| (knob in hole) | | | |
| | n = 3 donors | n = 2 donors | n = 3 donors |

TABLE 13
IL27RA and gp130 antibody sequences

| | IL27RA and gp130 antibody sequences | | | | | | | | | | | | | |
|---|---|---|---|---|---|---|---|---|---|---|---|---|---|---|
| | VH | VH | VH | VL | VL | VL | | | | | | | | |
| Antibody | CDR1 | CDR2 | CDR3 | CDR1 | CDR2 | CDR3 | VH | VL | CH1 | HINGE | CH2 | CH3 | HC | LC |
| Anti- | 1 | 2 | 3 | 4 | 5 | 6 | 7 | 8 | 9 | 10 | 11 | 12 | 13 | 14 |
| IL27RA- | | | | | | | | | | | | | | |
| 4701 | | | | | | | | | | | | | | |
| Anti- | 15 | 16 | 17 | 18 | 19 | 20 | 21 | 22 | 9 | 10 | 11 | 12 | 23 | 24 |
| gp130- | | | | | | | | | | | | | | |
| 4574 | | | | | | | | | | | | | | |
| Anti-IL- | 1 | 2 | 3 | 4 | 5 | 6 | 7 | 8 | 9 | 25 | 11 | 26 | 27 | 14 |
| 27RA- | | | | | | | | | | | | | | |
| 4880 | | | | | | | | | | | | | | |
| EE | | | | | | | | | | | | | | |
| Anti- | 15 | 16 | 17 | 18 | 19 | 20 | 21 | 22 | 9 | 28 | 11 | 29 | 30 | 24 |
| gp130- | | | | | | | | | | | | | | |
| 4875 | | | | | | | | | | | | | | |
| RR | | | | | | | | | | | | | | |

TABLE 14
IL27RA/gp130 bispecific antibody mAb-4894 sequences

| | IL27RA/gp130 bispecific antibody sequence (referred to herein as mAb-4894) | | | | | | | | | | | | | |
|---|---|---|---|---|---|---|---|---|---|---|---|---|---|---|
| Arms of | VH | VH | VH | VL | VL | VL | | | | | | | | |
| bispecific | CDR1 | CDR2 | CDR3 | CDR1 | CDR2 | CDR3 | VH | VL | CH1 | HINGE | CH2 | CH3 | HC | LC |
| First arm | 1 | 2 | 3 | 4 | 5 | 6 | 7 | 8 | 9 | 25 | 11 | 26 | 27 | 14 |
| from anti- | | | | | | | | | | | | | | |
| IL27RA- | | | | | | | | | | | | | | |
| 4880 EE | | | | | | | | | | | | | | |
| Second | 15 | 16 | 17 | 18 | 19 | 20 | 21 | 22 | 9 | 28 | 11 | 29 | 30 | 24 |
| arm from | | | | | | | | | | | | | | |
| anti- | | | | | | | | | | | | | | |
| gp130- | | | | | | | | | | | | | | |
| 4875 RR | | | | | | | | | | | | | | |

TABLE 15
antibody encoding sequences

| | IL27RA and gp130 antibody encoding sequences | | | |
|---|---|---|---|---|
| Antibody | VH | VL | HC | LC |
| Anti-IL27RA - 4701 | 31 | 32 | 33 | 34 |
| Anti-gp130-4574 | 35 | 36 | 37 | 38 |
| Anti-IL27RA-4880 EE | 31 | 32 | 39 | 34 |
| Anti-gp130-4875 RR | 35 | 36 | 40 | 38 |

TABLE 16
SEQUENCE LISTING

| SEQ | DESCRIPTION | SEQUENCE |
|---|---|---|
| 1 | Anti-IL27RA- | GFTFEYFGLQ |
| | 4701/Anti-IL- | |
| | 27RA-4880 EE | |
| | VH CDR1 | |
| 2 | Anti-IL27RA- | YTSSGGNYEYYEETLKG |
| | 4701/Anti-IL- | |
| | 27RA-4880 EE | |
| | VH CDR2 | |
| 3 | Anti-IL27RA- | TKTGDY |
| | 4701/Anti-IL- | |
| | 27RA-4880 EE | |
| | VH CDR3 | |
| 4 | Anti-IL27RA- | RASQDIGNSLT |
| | 4701/Anti-IL- | |
| | 27RA-4880 EE | |
| | VL CDR1 | |
| 5 | Anti-IL27RA- | GATGLAA |
| | 4701/Anti-IL- | |
| | 27RA-4880 EE | |
| | VL CDR2 | |
| 6 | Anti-IL27RA- | LQSKQYPYT |
| | 4701/Anti-IL- | |
| | 27RA-4880 EE | |
| | VL CDR3 | |
| 7 | Anti-IL27RA- | EVQLVESGGGLVQPGGSLRLSCAASGFTFEYFGLQWVRQAPGKGLEWIGYTSSGGNYEYYEE |
| | 4701/Anti-IL- | TLKGRFTISRDNAKNSLYLQMNSLRAEDTAVYYCARTKTGDYWGQGTLVTVSS |
| | 27RA-4880 EE | |
| | VH | |
| 8 | Anti-IL27RA- | DIQMTQSPSSLSASVGDRVTITCRASQDIGNSLTWYQQKPGKAPKRVIFGATGLAAGVPSRF |
| | 4701/Anti-IL- | SGSGSGTDFTLTISSLQPEDFATYYCLQSKQYPYTFGQGTKLEIK |
| | 27RA-4880 EE | |
| | VL | |
| 9 | CH1 | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSC |
| 10 | Anti-IL27RA- | DKTHTCPPCP APEAAGA |
| | 4701/Anti- | |
| | gp130-4574 | |
| | HINGE | |
| 11 | CH2 | PSVFLFPPKP KDTLMISRTP EVTCVVVDVS HEDPEVKENW YVDGVEVHNA |
| | | KTKPREEQYN STYRVVSVLT VLHQDWLNGK EYKCKVSNKA LPAPIEKTIS KAK |
| 12 | Anti-IL27RA- | GQPREPQVYT LPPSREEMTK NQVSLTCLVK GFYPSDIAVE WESNGQPENN |
| | 4701/Anti- | YKTTPPVLDS DGSFFLYSKL TVDKSRWQQG NVFSCSVMHE ALHNHYTQKS LSLSPGK |
| | gp130-4574 CH3 | |
| 13 | Anti-IL27RA- | EVQLVESGGG LVQPGGSLRL SCAASGFTFE YFGLQWVRQA PGKGLEWIGY |
| | 4701 HC | TSSGGNYEYY EETLKGRFTI SRDNAKNSLY LQMNSLRAED TAVYYCARTK |
| | | TGDYWGQGTL VTVSSASTKG PSVFPLAPSS KSTSGGTAAL GCLVKDYFPE |
| | | PVTVSWNSGA LTSGVHTFPA VLQSSGLYSL SSVVTVPSSS LGTQTYICNV |
| | | NHKPSNTKVD KKVEPKSCDK THTCPPCPAP EAAGAPSVFL FPPKPKDTLM |
| | | ISRTPEVTCV VVDVSHEDPE VKENWYVDGV EVHNAKTKPR EEQYNSTYRV |
| | | VSVLTVLHQD WINGKEYKCK VSNKALPAPI EKTISKAKGQ PREPQVYTLP |
| | | PSREEMTKNQ VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| 14 | Anti-IL27RA- | DIQMTQSPSS LSASVGDRVT ITCRASQDIG NSLTWYQQKP GKAPKRVIFG |
| | 4701/Anti-IL- | ATGLAAGVPS RFSGSGSGTD FTLTISSLQP EDFATYYCLQ SKQYPYTFGQ |
| | 27RA-4880 LC | GTKLEIKRTV AAPSVFIFPP SDEQLKSGTA SVVCLLNNFY PREAKVQWKV |
| | | DNALQSGNSQ ESVTEQDSKD STYSLSSTLT LSKADYEKHK VYACEVTHQG |
| | | LSSPVTKSEN RGEC |
| 15 | Anti-gp130- | GGSISSYYWS |
| | 4574/Anti- | |
| | gp130-4875 RR | |
| | VH CDR1 | |
| 16 | Anti-gp130- | RIYITGNTNY NPSLKD |
| | 4574/Anti- | |
| | gp130-4875 RR | |
| | VH CDR2 | |
| 17 | Anti-gp130- | EGPKLGIFDY |
| | 4574/Anti- | |
| | gp130-4875 RR | |
| | VH CDR3 | |
| 18 | Anti-gp130- | RASQSVSSSY LA |
| | 4574/Anti- | |
| | gp130-4875 RR | |
| | VL CDR1 | |
| 19 | Anti-gp130- | GTESRAT |
| | 4574/Anti- | |
| | gp130-4875 RR | |
| | VL CDR2 | |
| 20 | Anti-gp130- | QQYGGYPT |
| | 4574/Anti- | |
| | gp130-4875 RR | |
| | VL CDR3 | |
| 21 | Anti-gp130- | QVQLVQSGAE VKKPGSSVKV SCKVSGGSIS SYYWSWVRQA PGQGLEWIGR |
| | 4574/Anti- | IYITGNTNYN PSLKDRVTMT VDTSTSTHYM ELSSLRSEDT AVYYCAREGP |
| | gp130-4875 RR | KLGIFDYWGQ GTLVTVSS |
| | VH | |
| 22 | Anti-gp130- | DIQMTQSPSS LSASVGDRVT ITCRASQSVS SSYLAWYQQK PGKAPKLLIY |
| | 4574/Anti- | GTESRATGVP SRFSGSGSGT DETLTISSLQ PEDFATYYCQ QYGGYPTFGGGTKVEIK |
| | gp130-4875 RR | |
| | VL | |
| 23 | Anti-gp130- | QVQLVQSGAE VKKPGSSVKV SCKVSGGSIS SYYWSWVRQA PGQGLEWIGR |
| | 4574 HC | IYITGNTNYN PSLKDRVTMT VDTSTSTHYM ELSSLRSEDT AVYYCAREGP |
| | | KLGIFDYWGQ GTLVTVSSAS TKGPSVFPLA PSSKSTSGGT AALGCLVKDY |
| | | FPEPVTVSWN SGALTSGVHT FPAVLQSSGL YSLSSVVTVP SSSLGTQTYI |
| | | CNVNHKPSNT KVDKKVEPKS CDKTHTCPPC PAPEAAGAPS VFLFPPKPKD |
| | | TLMISRTPEV TCVVVDVSHE DPEVKENWYV DGVEVHNAKT KPREEQYNST |
| | | YRVVSVLTVL HQDWLNGKEY KCKVSNKALP APIEKTISKA KGQPREPQVY |
| | | TLPPSREEMT KNQVSLTCLV KGFYPSDIAV EWESNGQPEN NYKTTPPVLD |
| | | SDGSFFLYSK LTVDKSRWQQ GNVFSCSVMH EALHNHYTQK SLSLSPGK |
| 24 | Anti-gp130- | DIQMTQSPSS LSASVGDRVT ITCRASQSVS SSYLAWYQQK PGKAPKLLIY |
| | | GTESRATGVP SRFSGSGSGT DFTLTISSLQ PEDFATYYCQ QYGGYPTFGG |
| | 4574/Anti- | GTKVEIKRTV AAPSVFIFPP SDEQLKSGTA SVVCLLNNFY PREAKVQWKV |
| | gp130-4875 LC | DNALQSGNSQ ESVTEQDSKD STYSLSSTLT LSKADYEKHK VYACEVTHQG |
| | | LSSPVTKSEN RGEC |
| 25 | Anti-IL-27RA- | EKTHTCPPCP APEAAGA |
| | 4880 EE HINGE | |
| 26 | Anti-IL-27RA- | GQPREPQVYT LPPSREEMTK NQVSLTCEVK GFYPSDIAVE WESNGQPENN |
| | 4880 EE CH3 | YKTTPPVLDS DGSFFLYSKL TVDKSRWQQG NVFSCSVMHE ALHNHYTQKS LSLSPGK |
| 27 | Anti-IL-27RA- | EVQLVESGGG LVQPGGSLRL SCAASGFTFE YFGLQWVRQA PGKGLEWIGY |
| | 4880 EE HC | TSSGGNYEYY EETLKGRFTI SRDNAKNSLY LQMNSLRAED TAVYYCARTK |
| | | TGDYWGQGTL VTVSSASTKG PSVFPLAPSS KSTSGGTAAL GCLVKDYFPE |
| | | PVTVSWNSGA LTSGVHTFPA VLQSSGLYSL SSVVTVPSSS LGTQTYICNV |
| | | NHKPSNTKVD KKVEPKSCEK THTCPPCPAP EAAGAPSVFL FPPKPKDTLM |
| | | ISRTPEVTCV VVDVSHEDPE VKENWYVDGV EVHNAKTKPR EEQYNSTYRV |
| | | VSVLTVLHQD WINGKEYKCK VSNKALPAPI EKTISKAKGQ PREPQVYTLP |
| | | PSREEMTKNQ VSLTCEVKGF YPSDIAVEWE SNGQPENNYK TTPPVLDSDG |
| | | SFFLYSKLTV DKSRWQQGNV FSCSVMHEAL HNHYTQKSLS LSPGK |
| 28 | Anti-gp130- | RKTHTCPPCP APEAAGA |
| | 4875 RR HINGE | |
| 29 | Anti-gp130- | GQPREPQVYT LPPSREEMTK NQVSLTCLVK GFYPSDIAVE WESNGQPENN |
| | 4875 RR CH3 | YKTTPPVLDS DGSFFLYSRL TVDKSRWQQG NVFSCSVMHE ALHNHYTQKS LSLSPGK |
| 30 | Anti-gp130- | QVQLVQSGAE VKKPGSSVKV SCKVSGGSIS SYYWSWVRQA PGQGLEWIGR |
| | 4875 RR HC | IYITGNTNYN PSLKDRVTMT VDTSTSTHYM ELSSLRSEDT AVYYCAREGP |
| | | KLGIFDYWGQ GTLVTVSSAS TKGPSVFPLA PSSKSTSGGT AALGCLVKDY |
| | | FPEPVTVSWN SGALTSGVHT FPAVLQSSGL YSLSSVVTVP SSSLGTQTYI |
| | | CNVNHKPSNT KVDKKVEPKS CRKTHTCPPC PAPEAAGAPS VFLFPPKPKD |
| | | TLMISRTPEV TCVVVDVSHE DPEVKENWYV DGVEVHNAKT KPREEQYNST |
| | | YRVVSVLTVL HQDWLNGKEY KCKVSNKALP APIEKTISKA KGQPREPQVY |
| | | TLPPSREEMT KNQVSLTCLV KGFYPSDIAV EWESNGQPEN NYKTTPPVLD |
| | | SDGSFFLYSR LTVDKSRWQQ GNVFSCSVMH EALHNHYTQK SLSLSPGK |
| 31 | Anti-IL27RA- | GAGGTGCAGCTGGTGGAGTCTGGGGGAGGCTTGGTCCAGCCTGGGGGGTCCCTGAGACTCTC |
| | 4701/Anti-IL- | CTGTGCAGCCTCTGGATTCACCTTTGAGTACTTTGGATTGCAGTGGGTCCGCCAGGCTCCAG |
| | 27RA-4880 EE | GGAAGGGGCTGGAGTGGATTGGATACACCAGTAGTGGTGGAAATTACGAGTACTATGAAGAA |
| | VH DNA | ACGCTGAAGGGCCGATTCACCATCTCCAGAGACAACGCCAAGAACTCACTGTATCTGCAAAT |
| | | GAACAGCCTGAGAGCCGAGGACACGGCTGTGTATTACTGTGCGAGAACGAAGACCGGGGACT |
| | | ACTGGGGCCAAGGAACCCTGGTCACCGTCTCCTCA |
| 32 | Anti-IL27RA- | GACATCCAGATGACCCAGTCTCCATCCTCCCTGTCTGCATCTGTAGGAGACAGAGTCACCAT |
| | 4701/Anti-IL- | CACTTGCCGGGCAAGTCAGGACATTGGAAATTCTTTAACCTGGTATCAGCAGAAACCAGGGA |
| | 27RA-4880 EE | AAGCCCCTAAGCGTGTGATCTTTGGTGCAACCGGCTTGGCAGCTGGGGTCCCATCAAGGTTC |
| | VL DNA | AGTGGCAGTGGATCTGGGACAGATTTCACTCTCACCATCAGCAGTCTGCAACCTGAAGATTT |
| | | TGCAACTTACTACTGTCTACAGAGTAAACAGTATCCTTACACTTTTGGCCAGGGGACCAAGC |
| | | TGGAGATCAAA |
| 33 | Anti-IL27RA- | GAGGTGCAGCTGGTGGAGTCTGGGGGAGGCTTGGTCCAGCCTGGGGGGTCCCTGAGACTCTC |
| | 4701 HC DNA | CTGTGCAGCCTCTGGATTCACCTTTGAGTACTTTGGATTGCAGTGGGTCCGCCAGGCTCCAG |
| | | GGAAGGGGCTGGAGTGGATTGGATACACCAGTAGTGGTGGAAATTACGAGTACTATGAAGAA |
| | | ACGCTGAAGGGCCGATTCACCATCTCCAGAGACAACGCCAAGAACTCACTGTATCTGCAAAT |
| | | GAACAGCCTGAGAGCCGAGGACACGGCTGTGTATTACTGTGCGAGAACGAAGACCGGGGACT |
| | | ACTGGGGCCAAGGAACCCTGGTCACCGTCTCCTCAGCGTCGACCAAGGGCCCATCGGTCTTC |
| | | CCCCTGGCACCCTCCTCCAAGAGCACCTCTGGGGGCACAGCGGCCCTGGGCTGCCTGGTCAA |
| | | GGACTACTTCCCCGAACCGGTGACGGTGTCGTGGAACTCAGGCGCCCTGACCAGCGGCGTGC |
| | | ACACCTTCCCGGCTGTCCTACAGTCCTCAGGACTCTACTCCCTCAGCAGCGTGGTGACCGTG |
| | | CCCTCCAGCAGCTTGGGCACCCAGACCTACATCTGCAACGTGAATCACAAGCCCAGCAACAC |
| | | CAAGGTGGACAAGAAAGTTGAGCCCAAATCTTGTGACAAAACTCACACATGCCCACCGTGCC |
| | | CAGCACCTGAAGCCGCTGGGGCACCGTCAGTCTTCCTCTTCCCCCCAAAACCCAAGGACACC |
| | | CTCATGATCTCCCGGACCCCTGAGGTCACATGCGTGGTGGTGGACGTGAGCCACGAAGACCC |
| | | TGAGGTCAAGTTCAACTGGTACGTGGACGGCGTGGAGGTGCATAATGCCAAGACAAAGCCGC |
| | | GGGAGGAGCAGTACAACAGCACGTACCGTGTGGTCAGCGTCCTCACCGTCCTGCACCAGGAC |
| | | TGGCTGAATGGCAAGGAGTACAAGTGCAAGGTCTCCAACAAAGCCCTCCCAGCCCCCATCGA |
| | | GAAAACCATCTCCAAAGCCAAAGGGCAGCCCCGAGAACCACAGGTGTACACCCTGCCCCCAT |
| | | CCCGGGAGGAGATGACCAAGAACCAGGTCAGCCTGACCTGCCTGGTCAAAGGCTTCTATCCC |
| | | AGCGACATCGCCGTGGAGTGGGAGAGCAATGGGCAGCCGGAGAACAACTACAAGACCACGCC |
| | | TCCCGTGCTGGACTCCGACGGCTCCTTCTTCCTCTATAGCAAGCTCACCGTGGACAAGAGCA |
| | | GGTGGCAGCAGGGGAACGTCTTCTCATGCTCCGTGATGCATGAGGCTCTGCACAACCACTAC |
| | | ACGCAGAAGAGCCTCTCCCTGTCCCCGGGAAAA |
| 34 | Anti-IL27RA- | GACATCCAGATGACCCAGTCTCCATCCTCCCTGTCTGCATCTGTAGGAGACAGAGTCACCAT |
| | 4701/Anti-IL- | CACTTGCCGGGCAAGTCAGGACATTGGAAATTCTTTAACCTGGTATCAGCAGAAACCAGGGA |
| | 27RA-4880 EE | AAGCCCCTAAGCGTGTGATCTTTGGTGCAACCGGCTTGGCAGCTGGGGTCCCATCAAGGTTC |
| | LC DNA | AGTGGCAGTGGATCTGGGACAGATTTCACTCTCACCATCAGCAGTCTGCAACCTGAAGATTT |
| | | TGCAACTTACTACTGTCTACAGAGTAAACAGTATCCTTACACTTTTGGCCAGGGGACCAAGC |
| | | TGGAGATCAAACGTACGGTGGCTGCACCATCTGTCTTCATCTTCCCGCCATCTGATGAGCAG |
| | | TTGAAATCTGGAACTGCCTCTGTTGTGTGCCTGCTGAATAACTTCTATCCCAGAGAGGCCAA |
| | | AGTACAGTGGAAGGTGGATAACGCCCTCCAATCGGGTAACTCCCAGGAGAGTGTCACAGAGC |
| | | AGGACAGCAAGGACAGCACCTACAGCCTCAGCAGCACCCTGACGCTGAGCAAAGCAGACTAC |
| | | GAGAAACACAAAGTCTACGCCTGCGAAGTCACCCATCAGGGCCTGAGCTCGCCCGTCACAAA |
| | | GAGCTTCAACAGGGGAGAGTGT |
| 35 | Anti-gp130- | CAGGTGCAGCTGGTGCAGTCTGGGGCTGAGGTGAAGAAGCCTGGGTCCTCGGTGAAGGTCTC |
| | 4574/Anti- | CTGCAAGGTCTCTGGAGGCTCCATCAGCAGCTATTACTGGAGCTGGGTGCGACAGGCCCCTG |
| | gp130-4875 RR | GACAAGGGCTTGAGTGGATTGGACGTATCTATATCACCGGTAACACCAACTACAACCCCTCC |
| | VH DNA | CTCAAGGACAGAGTCACGATGACCGTAGACACGTCCACGAGCACACACTACATGGAGCTGAG |
| | | CAGCCTGAGATCTGAGGACACGGCCGTGTATTACTGTGCGAGAGAAGGGCCCAAACTGGGGA |
| | | TCTTTGACTACTGGGGCCAAGGAACCCTGGTCACCGTCTCCTCA |
| 36 | Anti-gp130- | GACATCCAGATGACCCAGTCTCCATCCTCCCTGTCTGCATCTGTAGGAGACAGAGTCACCAT |
| | 4574/Anti- | CACTTGCCGGGCAAGTCAGAGTGTTAGCAGCAGCTATTTAGCCTGGTATCAGCAGAAACCAG |
| | gp130-4875 RR | GGAAAGCCCCTAAGCTCCTGATCTATGGTACAGAGAGTAGGGCCACTGGGGTCCCATCAAGG |
| | VL DNA | TTCAGTGGCAGTGGATCTGGGACAGATTTCACTCTCACCATCAGCAGTCTGCAACCTGAAGA |
| | | TTTTGCAACTTACTACTGTCAACAGTATGGTGGCTACCCTACTTTCGGCGGAGGGACCAAGG |
| | | TGGAGATCAAA |
| 37 | Anti-gp130- | CAGGTGCAGCTGGTGCAGTCTGGGGCTGAGGTGAAGAAGCCTGGGTCCTCGGTGAAGGTCTC |
| | 4574 HC DNA | CTGCAAGGTCTCTGGAGGCTCCATCAGCAGCTATTACTGGAGCTGGGTGCGACAGGCCCCTG |
| | | GACAAGGGCTTGAGTGGATTGGACGTATCTATATCACCGGTAACACCAACTACAACCCCTCC |
| | | CTCAAGGACAGAGTCACGATGACCGTAGACACGTCCACGAGCACACACTACATGGAGCTGAG |
| | | CAGCCTGAGATCTGAGGACACGGCCGTGTATTACTGTGCGAGAGAAGGGCCCAAACTGGGGA |
| | | TCTTTGACTACTGGGGCCAAGGAACCCTGGTCACCGTCTCCTCAGCGTCGACCAAGGGCCCA |
| | | TCGGTCTTCCCCCTGGCACCCTCCTCCAAGAGCACCTCTGGGGGCACAGCGGCCCTGGGCTG |
| | | CCTGGTCAAGGACTACTTCCCCGAACCGGTGACGGTGTCGTGGAACTCAGGCGCCCTGACCA |
| | | GCGGCGTGCACACCTTCCCGGCTGTCCTACAGTCCTCAGGACTCTACTCCCTCAGCAGCGTG |
| | | GTGACCGTGCCCTCCAGCAGCTTGGGCACCCAGACCTACATCTGCAACGTGAATCACAAGCC |
| | | CAGCAACACCAAGGTGGACAAGAAAGTTGAGCCCAAATCTTGTGACAAAACTCACACATGCC |
| | | CACCGTGCCCAGCACCTGAAGCCGCTGGGGCACCGTCAGTCTTCCTCTTCCCCCCAAAACCC |
| | | AAGGACACCCTCATGATCTCCCGGACCCCTGAGGTCACATGCGTGGTGGTGGACGTGAGCCA |
| | | CGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGTGTGGTCAGCGTCCTCACCGTCCTG |
| | | CACCAGGACTGGCTGAATGGCAAGGAGTACAAGTGCAAGGTCTCCAACAAAGCCCTCCCAGC |
| | | CCCCATCGAGAAAACCATCTCCAAAGCCAAAGGGCAGCCCCGAGAACCACAGGTGTACACCC |
| | | TGCCCCCATCCCGGGAGGAGATGACCAAGAACCAGGTCAGCCTGACCTGCCTGGTCAAAGGC |
| | | TTCTATCCCAGCGACATCGCCGTGGAGTGGGAGAGCAATGGGCAGCCGGAGAACAACTACAA |
| | | GACCACGCCTCCCGTGCTGGACTCCGACGGCTCCTTCTTCCTCTATAGCAAGCTCACCGTGG |
| | | ACAAGAGCAGGTGGCAGCAGGGGAACGTCTTCTCATGCTCCGTGATGCATGAGGCTCTGCAC |
| | | AACCACTACACGCAGAAGAGCCTCTCCCTGTCCCCGGGAAAA |
| 38 | Anti-gp130- | GACATCCAGATGACCCAGTCTCCATCCTCCCTGTCTGCATCTGTAGGAGACAGAGTCACCAT |
| | 4574/ Anti- | CACTTGCCGGGCAAGTCAGAGTGTTAGCAGCAGCTATTTAGCCTGGTATCAGCAGAAACCAG |
| | gp130-4875 RR | GGAAAGCCCCTAAGCTCCTGATCTATGGTACAGAGAGTAGGGCCACTGGGGTCCCATCAAGG |
| | LC DNA | TTCAGTGGCAGTGGATCTGGGACAGATTTCACTCTCACCATCAGCAGTCTGCAACCTGAAGA |
| | | TTTTGCAACTTACTACTGTCAACAGTATGGTGGCTACCCTACTTTCGGCGGAGGGACCAAGG |
| | | TGGAGATCAAACGTACGGTGGCTGCACCATCTGTCTTCATCTTCCCGCCATCTGATGAGCAG |
| | | TTGAAATCTGGAACTGCCTCTGTTGTGTGCCTGCTGAATAACTTCTATCCCAGAGAGGCCAA |
| | | AGTACAGTGGAAGGTGGATAACGCCCTCCAATCGGGTAACTCCCAGGAGAGTGTCACAGAGC |
| | | AGGACAGCAAGGACAGCACCTACAGCCTCAGCAGCACCCTGACGCTGAGCAAAGCAGACTAC |
| | | GAGAAACACAAAGTCTACGCCTGCGAAGTCACCCATCAGGGCCTGAGCTCGCCCGTCACAAA |
| | | GAGCTTCAACAGGGGAGAGTGT |
| 39 | Anti-IL-27RA- | GAGGTGCAGCTGGTGGAGTCTGGGGGAGGCTTGGTCCAGCCTGGGGGGTCCCTGAGACTCTC |
| | 4880 EE HC DNA | CTGTGCAGCCTCTGGATTCACCTTTGAGTACTTTGGATTGCAGTGGGTCCGCCAGGCTCCAG |
| | | GGAAGGGGCTGGAGTGGATTGGATACACCAGTAGTGGTGGAAATTACGAGTACTATGAAGAA |
| | | ACGCTGAAGGGCCGATTCACCATCTCCAGAGACAACGCCAAGAACTCACTGTATCTGCAAAT |
| | | GAACAGCCTGAGAGCCGAGGACACGGCTGTGTATTACTGTGCGAGAACGAAGACCGGGGACT |
| | | ACTGGGGCCAAGGAACCCTGGTCACCGTCTCCTCAGCGTCGACCAAGGGCCCATCGGTCTTC |
| | | CCCCTGGCACCCTCCTCCAAGAGCACCTCTGGGGGCACAGCGGCCCTGGGCTGCCTGGTCAA |
| | | GGACTACTTCCCCGAACCGGTGACGGTGTCGTGGAACTCAGGCGCCCTGACCAGCGGCGTGC |
| | | ACACCTTCCCGGCTGTCCTACAGTCCTCAGGACTCTACTCCCTCAGCAGCGTGGTGACCGTG |
| | | CCCTCCAGCAGCTTGGGCACCCAGACCTACATCTGCAACGTGAATCACAAGCCCAGCAACAC |
| | | CAAGGTGGACAAGAAAGTTGAGCCCAAATCTTGTGAGAAAACTCACACATGCCCACCGTGCC |
| | | CAGCACCTGAAGCCGCTGGGGCACCGTCAGTCTTCCTCTTCCCCCCAAAACCCAAGGACACC |
| | | CTCATGATCTCCCGGACCCCTGAGGTCACATGCGTGGTGGTGGACGTGAGCCACGAAGACCC |
| | | TGAGGTCAAGTTCAACTGGTACGTGGACGGCGTGGAGGTGCATAATGCCAAGACAAAGCCGC |
| | | GGGAAGAGCAGTACAACAGCACGTACCGTGTGGTCAGCGTCCTCACCGTCCTGCACCAGGAC |
| | | TGGCTGAATGGCAAGGAGTACAAGTGCAAGGTCTCCAACAAAGCCCTCCCAGCCCCCATCGA |
| | | GAAAACCATCTCCAAAGCCAAAGGGCAGCCCCGAGAACCACAGGTGTACACCCTGCCCCCAT |
| | | CCCGGGAGGAGATGACCAAGAACCAGGTCAGCCTGACCTGCGAGGTCAAAGGCTTCTATCCC |
| | | AGCGACATCGCCGTGGAGTGGGAGAGCAATGGGCAGCCGGAGAACAACTACAAGACCACGCC |
| | | TCCCGTGCTGGACTCCGACGGCTCCTTCTTCCTCTATAGCAAGCTCACCGTGGACAAGAGCA |
| | | GGTGGCAGCAGGGGAACGTCTTCTCATGCTCCGTGATGCATGAGGCTCTGCACAACCACTAC |
| | | ACGCAGAAGAGCCTCTCCCTGTCCCCGGGAAAA |
| 40 | Anti-gp130- | CAGGTGCAGCTGGTGCAGTCTGGGGCTGAGGTGAAGAAGCCTGGGTCCTCGGTGAAGGTCTC |
| | 4875 RR HC DNA | CTGCAAGGTCTCTGGAGGCTCCATCAGCAGCTATTACTGGAGCTGGGTGCGACAGGCCCCTG |
| | | GACAAGGGCTTGAGTGGATTGGACGTATCTATATCACCGGTAACACCAACTACAACCCCTCC |
| | | CTCAAGGACAGAGTCACGATGACCGTAGACACGTCCACGAGCACACACTACATGGAGCTGAG |
| | | CAGCCTGAGATCTGAGGACACGGCCGTGTATTACTGTGCGAGAGAAGGGCCCAAACTGGGGA |
| | | TCTTTGACTACTGGGGCCAAGGAACCCTGGTCACCGTCTCCTCAGCGTCGACCAAGGGCCCA |
| | | TCGGTCTTCCCCCTGGCACCCTCCTCCAAGAGCACCTCTGGGGGCACAGCGGCCCTGGGCTG |
| | | CCTGGTCAAGGACTACTTCCCCGAACCGGTGACGGTGTCGTGGAACTCAGGCGCCCTGACCA |
| | | GCGGCGTGCACACCTTCCCGGCTGTCCTACAGTCCTCAGGACTCTACTCCCTCAGCAGCGTG |
| | | GTGACCGTGCCCTCCAGCAGCTTGGGCACCCAGACCTACATCTGCAACGTGAATCACAAGCC |
| | | CAGCAACACCAAGGTGGACAAGAAAGTTGAGCCCAAATCTTGTCGGAAAACTCACACATGCC |
| | | CACCGTGCCCAGCACCTGAAGCCGCTGGGGCACCGTCAGTCTTCCTCTTCCCCCCAAAACCC |
| | | AAGGACACCCTCATGATCTCCCGGACCCCTGAGGTCACATGCGTGGTGGTGGACGTGAGCCA |
| | | CGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGTGTGGTCAGCGTCCTCACCGTCCTG |
| | | CACCAGGACTGGCTGAATGGCAAGGAGTACAAGTGCAAGGTCTCCAACAAAGCCCTCCCAGC |
| | | CCCCATCGAGAAAACCATCTCCAAAGCCAAAGGGCAGCCCCGAGAACCACAGGTGTACACCC |
| | | TGCCCCCATCCCGGGAGGAGATGACCAAGAACCAGGTCAGCCTGACCTGCCTGGTCAAAGGC |
| | | TTCTATCCCAGCGACATCGCCGTGGAGTGGGAGAGCAATGGGCAGCCGGAGAACAACTACAA |
| | | GACCACGCCTCCCGTGCTGGACTCCGACGGCTCCTTCTTCCTCTATAGCAGGCTCACCGTGG |
| | | ACAAGAGCAGGTGGCAGCAGGGGAACGTCTTCTCATGCTCCGTGATGCATGAGGCTCTGCAC |
| | | AACCACTACACGCAGAAGAGCCTCTCCCTGTCCCCGGGAAAA |
| 41 | IL27RA human | MRGGRGAPFW LWPLPKLALL PLLWVLFQRT RPQGSAGPLQ CYGVGPLGDL |
| | | NCSWEPLGDL GAPSELHLQS QKYRSNKTQT VAVAAGRSWV AIPREQLTMS |
| | | DKLLVWGTKA GQPLWPPVFV NLETQMKPNA PRLGPDVDFS EDDPLEATVH |
| | | WAPPTWPSHK VLICQFHYRR CQEAAWTLLE PELKTIPLTP VEIQDLELAT |
| | | GYKVYGRCRM EKEEDLWGEW SPILSFQTPP SAPKDVWVSG NLCGTPGGEE |
| | | PLLLWKAPGP CVQVSYKVWF WVGGRELSPE GITCCCSLIP SGAEWARVSA |
| | | VNATSWEPLT NLSLVCLDSA SAPRSVAVSS IAGSTELLVT WQPGPGEPLE |
| | | HVVDWARDGD PLEKLNWVRL PPGNLSALLP GNFTVGVPYR ITVTAVSASG |
| | | LASASSVWGF REELAPLVGP TLWRLQDAPP GTPAIAWGEV PRHQLRGHLT |
| | | HYTLCAQSGT SPSVCMNVSG NTQSVTLPDL PWGPCELWVT ASTIAGQGPP |
| | | GPILRLHLPD NTLRWKVLPG ILFLWGLFLL GCGLSLATSG RCYHLRHKVL |
| | | PRWVWEKVPD PANSSSGQPH MEQVPEAQPL GDLPILEVEE MEPPPVMESS |
| | | QPAQATAPLD SGYEKHELPT PEELGLLGPP RPQVLA |
| 42 | IL27RA | MRGGRGAPFW PWPLPKLALL PLLWLLFQRT RPQGSAGPLQ CYGVGPLGDL |
| | cynomolgus | NCSWEPLGDL GAPSELHFQS QKYRSNKTQT VAVAAGQSWV AIPREQLTMS |
| | | DKLLVWGTKA GQPLWTPIFV NLETQMKPNA PQLGPDVDVS EDEPLEAIVH |
| | | WAPPTWPSHK VLICQFHYRR CQEAAWTLLE LELKTIPLAP VEIQDLELAT |
| | | GYQVYGRCRM EKEEDLWGEW SPILSFQTPP AAPKDVWVSG NLCGTPGGEE |
| | | PLLLWKAPGP CVQVSYKVWF WVGGRELSPE GVTCCGSLIP SGAEWARVSA |
| | | INATSWEPLT NLSLVCLDSA SAPHSVAVSS IAGSTELLVT WRPGPGEPRE |
| | | HVVDWTRDGD PLEKLNWVRL PPGNFSALLP GNFTVGVPYR ITVTAVSASG |
| | | LAPAPSIWGF REELAPLVGP TLWRLQDAPP GTPAIAWGEV PRHQLRGHLT |
| | | HYTLCAQGGT SPSVCMNVSG NTQSVTLPDL PWGPCELWVT ASTIAGQGPP |
| | | GPILRLHLPD NTLRWKVLPG ILFLWGLFLM GCGLSLATSG RCYHLWHKVL |
| | | PRWVWEKVPD PANSSSGQPH MEQVPEAQPL ADLPILEVEE MEPPPVMESS |
| | | QPPQVTAPLD SGYEKHELPT PEELGLLGTL RPQVLA |
| 43 | IL27RA rat | MSWLRVARLK PLELLLPLLP LLLGTRPHGS PGPLQCYSVG PLGILNCSWE |
| | | PLGDLETPPV LYHQSQKYHP NRTKEVKVPS RQSWVTIPRK QFTTADELLI |
| | | WGTQEGQLLW PPVSVNLETQ MKPDTPQIFS DVDFFKEETL EAIVQWEPPK |
| | | WPLHKVLICQ FRYKKCQTKE WTQLEPQLKT DLMTPIEMQN LEPGTGYQVS |
| | | GRCQVENGYP WGEWSPDLSF PTPFLAPEDV WVSGTICETS GKPAALLFWK |
| | | DPGPCVQVTY TVWLGVGDVT TTQEEVPCCK SPIPAQIEWA VVSTGNQTSW |
| | | MPLTNLSLVC LAPESAPCDV RVSSTDGSPG IKVTWKQETG KPWAYVVDWA |
| | | QDGDSLDKLN WTRLPPENLS TLLSGDFQAG VPYRITVTSV YSGGLAAAPS |
| | | VWGFIKELAP LAGPAVWRLP DDPTGTPVVA WGEVPRHQLR GQATHYTFCR |
| | | QSRGIPTICM NVSSQTKTVT LPNLYSGPFK LWVMVSTAAG QGPPGPSLSL |
| | | HIPDNRIRWK FLPWVLSLWG LLLVGCGLSL APPRCLYQRH KLLPQWIWER |
| | | VPDPANSNSG QPYIKEVSLP QPPKDGPILE VEEMELQPVT ESPKASVPLY |
| | | SGYEKHELPT PEELGLLRPP APRVLDCSAS G |
| 44 | IL27RA mouse | MNRLRVARLT PLELLLSLMS LLLGTRPHGS PGPLQCYSVG PLGILNCSWE |
| | | PLGDLETPPV LYHQSQKYHP NRVWEVKVPS KQSWVTIPRE QFTMADKLLI |
| | | WGTQKGRPLW SSVSVNLETQ MKPDTPQIFS QVDISEEATL EATVQWAPPV |
| | | WPPQKVLICQ FRYKECQAET WTRLEPQLKT DGLTPVEMQN LEPGTCYQVS |
| | | GRCQVENGYP WGEWSSPLSF QTPFLDPEDV WVSGTVCETS GKRAALLVWK |
| | | DPRPCVQVTY TVWFGAGDIT TTQEEVPCCK SPVPAWMEWA VVSPGNSTSW |
| | | VPPTNLSLVC LAPESAPCDV GVSSADGSPG IKVTWKQGTR KPLEYVVDWA |
| | | QDGDSLDKLN WTRLPPGNLS TLLPGEFKGG VPYRITVTAV YSGGLAAAPS |
| | | VWGFREELVP LAGPAVWRLP DDPPGTPVVS WGEVPHRQLR GQATHYTFCI |
| | | QSRGLSTVCR NVSSQTQTAT LPNLHLGSFK LWVTVSTVAG QGPPGPNLSL |
| | | HLPDNRIRWK ALPWFLSLWG LLLMGCGLSL ASTRCLQARC LHWRHKLLPQ |
| | | WIWERVPDPA NSNSGQPYIK EVSLPQPPKD GPILEVEEVE LQPVVESPKA |
| | | SAPIYSGYEK HELPTPEELG LLV |
| 45 | gp130 human | MLTLQTWLVQ ALFIFLTTES TGELLDPCGY ISPESPVVQL HSNFTAVCVL |
| | | KEKCMDYFHV NANYIVWKTN HFTIPKEQYT IINRTASSVT FTDIASLNIQ |
| | | LTCNILTFGQ LEQNVYGITI ISGLPPEKPK NLSCIVNEGK KMRCEWDGGR |
| | | ETHLETNFTL KSEWATHKFA DCKAKRDTPT SCTVDYSTVY FVNIEVWVEA |
| | | ENALGKVTSD HINFDPVYKV KPNPPHNLSV INSEELSSIL KLTWINPSIK |
| | | SVIILKYNIQ YRTKDASTWS QIPPEDTAST RSSFTVQDLK PFTEYVERIR |
| | | CMKEDGKGYW SDWSEEASGI TYEDRPSKAP SFWYKIDPSH TQGYRTVQLV |
| | | WKTLPPFEAN GKILDYEVTL TRWKSHLQNY TVNATKLTVN LTNDRYLATL |
| | | TVRNLVGKSD AAVLTIPACD FQATHPVMDL KAFPKDNMLW VEWTTPRESV |
| | | KKYILEWCVL SDKAPCITDW QQEDGTVHRT YLRGNLAESK CYLITVTPVY |
| | | ADGPGSPESI KAYLKQAPPS KGPTVRTKKV GKNEAVLEWD QLPVDVQNGE |
| | | IRNYTIFYRT IIGNETAVNV DSSHTEYTLS SLTSDTLYMV RMAAYTDEGG |
| | | KDGPEFTETT PKFAQGEIEA IVVPVCLAFL LTTLLGVLFC FNKRDLIKKH |
| | | IWPNVPDPSK SHIAQWSPHT PPRHNFNSKD QMYSDGNFTD VSVVEIEAND |
| | | KKPFPEDLKS LDLFKKEKIN TEGHSSGIGG SSCMSSSRPS ISSSDENESS |
| | | QNTSSTVQYS TVVHSGYRHQ VPSVQVFSRS ESTQPLLDSE ERPEDLQLVD |
| | | HVDGGDGILP RQQYFKQNCS QHESSPDISH FERSKQVSSV NEEDFVRLKQ |
| | | QISDHISQSC GSGQMKMFQE VSAADAFGPG TEGQVERFET VGMEAATDEG |
| | | MPKSYLPQTV RQGGYMPQ |
| 46 | gp130 | MLTLQTWVVQ ALFIFLTTES IGELLDPCGY ISPESPVVQL HSNFTAVCVL |
| | cynomolgus | KEKCMDYFHV NANYIVWKTN HFTIPKEQYT IINRTASSVT FTDISSLNIQ |
| | | LTCNILTFGQ LEQNVYGITI ISGLPPEKPK NLSCIVNEGK KMRCEWNRGR |
| | | ETHLETNFTL KSEWATHKFA DCKAKRDTPT SCTVDYSTVY FVNIEVWVEA |
| | | ENALGKVTSD HINFDPVYKV KPNPPHNLSV INSEELSSIL KLTWINPSIK |
| | | SVIRLKYNIQ YRTKDASTWS QIPPEDTAST RSSFTVQDLK PFTEYVFRIC |
| | | CMKEDGKGYW SDWSEEANGI TYEDRPSKAP SFWYKIDPSH AQGYRTVQLM |
| | | WKTLPPFEAN GKILDYEVTL TRWKSHLQNY TVNDTKLTVN LTNDRYVATL |
| | | TARNLVGKSD AAVLTIPACD FQATHPVMDL KAFPKDNMLW VEWTTPRESV |
| | | KKYILEWCVL SDKAPCIADW QQEDGTVHRT HLRGNLAESK CYLITVTPVY |
| | | ADGPGSPESI KAYLKQAPPS KGPTVRTKKV GKNEAVLEWD QLPVDVQNGF |
| | | IRNYTIFYRT IIGNETAVNV DSSHTEYTLS SLTSDTLYMV RMAAYTDEGG |
| | | KDGPEFTFTT PKFAQGEIEA IVVPVCLAFL LTTLLGVLFC FNKRDLIKKH |
| | | IWPNVPDPSK SHIAQWSPHT PPRHNFSSKD QMYSDGNFTD VSVVEIEAND |
| | | KKPFPEDLKS LDLFKKEKIN TEGHSSGIGG SSCMSSSRPS ISSSDENESS |
| | | QNTSSTVQYS TVVHSGYRHQ VPSVQVFSRS ESTQPLLDSE ERPEDLQLVD |
| | | HVDGSDDILP RQQYFKQNCS QHESSPDISH FERSKQVSSV NEEDFVRLKQ |
| | | QISDHISQSC GSGEMKMFQE VSAADPFGPG TEGQVERFET IGMEAAIDEG |
| | | MPKSYLPQTV RQGGYMPQ |
| 47 | gp130 rat | MSALRIWLMQ ALLIFLTTES IGQLVEPCGY IYPEFPVVQR GSNFTATCVL |
| | | KEKCLQVYSV NATYIVWKTN HVAVPKEQVT VINRTASSVT FTDVVFQNVQ |
| | | LTCNILSFGQ IEQNVYGITI LSGYPPDIPT NLSCIVNEGK NMLCQWDPGR |
| | | ETYLETNYTL KSEWATEKFP DCRTKHGTSS CMMGYTPIYF VNIEVWVEAE |
| | | NALGNVSSEP INFDPVDKVK PSPPHNLSVT NSEELSSILK LAWVNSGLDS |
| | | ILRLKSDIQY RTKDASTWIQ VPLEDTVSPR TSFTVQDLKP FTEYVFRIRS |
| | | IKENGKGYWS DWSEEASGTT YEDRPSKAPS FWYKVNANHP QEYRSARLIW |
| | | KTLPLSEANG KILDYEVVLT QSKSVSQTYT VNGTELIVNL TNNRYVASLA |
| | | ARNVVGKSPA TVLTIPGSHF KASHPVVDLK AFPKDNLLWV EWTPPSKPVN |
| | | KYILEWCVLS ENSPCIPDWQ QEDGTVNRTH LRGSLLESKC YLITVTPVFP |
| | | GGPGSPESMK AYLKQAAPSK GPTVRTKKVG KNEAVLEWDH LPVDVQNGFI |
| | | RNYSISYRTS VGKEMVVRVD SSHTEYTLSS LSSDTLYMVH MAAYTEEGGK |
| | | DGPEFTFTTL KFAQGEIEAI VVPVCLAFLL TTLLGVLFCF NKRDLIKKHI |
| | | WPNVPDPSKS HIAQWSPHTP PRHNENSKDQ MYSDANFTDV SVVEIEANNK |
| | | KPCPDDLKSL DLFKKEKIST EGHSSGIGGS SCMSSSRPSI SSSEENESAQ |
| | | STASTVQYST VVHSGYRHQV PSVQVFSRSE STQPLLDSEE RPEDLQLVDS |
| | | VDSGDEILPR QQYFKQSCSQ PGASPDVSHF ERSSQVPSGS EEDFVRLKQQ |
| | | QVSDHISEPY GSEQRRLFQE GSVADALGTG TDGQIERFES VGMETAMDED |
| | | ISKSYLPQTV RQGGYMPQ |
| 48 | gp130 mouse | MSAPRIWLAQ ALLFELTTES KEACLQHYYV NASYIVWKTN HAAVPREQVT |
| | | KEACLQHYYV NASYIVWKTN HAAVPREQVT VINRTTSSVT FTDVVLPSVQ |
| | | LTCNILSFGQ IEQNVYGVTM LSGFPPDKPT NLTCIVNEGK NMLCQWDPGR |
| | | ETYLETNYTL KSEWATEKFP DCQSKHGTSC MVSYMPTYYV NIEVWVEAEN |
| | | ALGKVSSESI NFDPVDKVKP TPPYNLSVTN SEELSSILKL SWVSSGLGGL |
| | | LDLKSDIQYR TKDASTWIQV PLEDTMSPRT SFTVQDLKPF TEYVFRIRSI |
| | | KDSGKGYWSD WSEEASGTTY EDRPSRPPSF WYKTNPSHGQ EYRSVRLIWK |
| | | ALPLSEANGK ILDYEVILTQ SKSVSQTYTV TGTELTVNLT NDRYVASLAA |
| | | RNKVGKSAAA VLTIPSPHVT AAYSVVNLKA FPKDNLLWVE WTPPPKPVSK |
| | | YILEWCVLSE NAPCVEDWQQ EDATVNRTHL RGRLLESKCY QITVTPVFAT |
| | | GPGGSESLKA YLKQAAPARG PTVRTKKVGK NEAVLAWDQI PVDDQNGFIR |
| | | NYSISYRTSV GKEMVVHVDS SHTEYTLSSL SSDTLYMVRM AAYTDEGGKD |
| | | GPEFTFTTPK FAQGEIEAIV VPVCLAFLLT TLLGVLFCEN KRDLIKKHIW |
| | | PNVPDPSKSH IAQWSPHTPP RHNENSKDQM YSDGNFTDVS VVEIEANNKK |
| | | PCPDDLKSVD LFKKEKVSTE GHSSGIGGSS CMSSSRPSIS SNEENESAQS |
| | | TASTVQYSTV VHSGYRHQVP SVQVFSRSES TQPLLDSEER PEDLQLVDSV |
| | | DGGDEILPRQ PYFKQNCSQP EACPEISHFE RSNQVLSGNE EDFVRLKQQQ |
| | | VSDHISQPYG SEQRRLFQEG STADALGTGA DGQMERFESV GMETTIDEEI |
| | | PKSYLPQTVR QGGYMPQ |
| 49 | IL27 p28 | FPRPPGRPQL SLQELRREFT VSLHLARKLL SEVRGQAHRF AESHLPGVNL |
| | monoFc | YLLPLGEQLP DVSLTFQAWR RLSDPERLSF ISTTLQPFHA LLGGLGTQGR |
| | | WTNMERMQLW AMRLDLRDLQ RHLRFQVLAA GENLPEEEEE EEEEEEEERK |
| | | GLLPGALGSA LQGPAQVSWP QLLSTYRLLH SLECVLSRAV RELLLLSKAG |
| | | HSVWPLGFPT LSPQPGGGGS TGGGPSVFLF PPKPKDTLMI SRTPEVTCVV |
| | | VDVSHEDPEV KENWYVDGVE VHNAKTKPRE EQYNSTYRVV SVLTVLHQDW |
| | | LNGKEYKCKV SNKALPAPIE KTISKAKGQP REPQVYTLPP SREEMTKNQV |
| | | NLTCLVKGFY PSDIAVEWES NGQPENNYKT TPPVLDSDGS FFLNSTLTVD |
| | | KSRWQQGNVF SCSVLHEALH SHYTQKSLSL SPKHHHHHH |
| 50 | EBI3 | RKGPPAALTL PRVQCRASRY PIAVDCSWTL PPAPNSTSPV SFIATYRLGM |
| | | AARGHSWPCL QQTPTSTSCT ITDVQLFSCA PYVLNVTAVH PWGSSSSFVP |
| | | FITEHIIKPD PPEGVRLSPL AERQLQVQWE PPGSWPFPEI FSLKYWIRYK |
| | | RQGAARFHRV GPIEATSFIL RAVRPRARYY VQVAAQDLTD YGELSDWSLP |
| | | ATATMSLGKG SGDYKDDDDK |
| 51 | IL27 p28 Fc | FPRPPGRPQL SLQELRREFT VSLHLARKLL SEVRGQAHRF AESHLPGVNL |
| | Knob mutations | YLLPLGEQLP DVSLTFQAWR RLSDPERLSF ISTTLQPFHA LLGGLGTQGR |
| | Y349C-T366W | WTNMERMQLW AMRLDLRDLQ RHLRFQVLAA GENLPEEEEE EEEEEEEERK |
| | bold and | GLLPGALGSA LQGPAQVSWP QLLSTYRLLH SLECVLSRAV RELLLLSKAG |
| | underlined, Fc | HSVWPLGFPT LSPQPENLYF QGVTGEPKSS DKTHTCPPCP APELLGGPSV |
| | portion | FLFPPKPKDT LMISRTPEVT CVVVDVSHED PEVKENWYVD GVEVHNAKTK |
| | italics. | PREEQYNSTY RVVSVLTVLH QDWLNGKEYK CKVSNKALPA PIEKTISKAK |
| | | GQPREPQV [symbol] T LPPSREEMTK NQVSL [symbol] CLVK GFYPSDIAVE WESNGQPENN |
| | | YKTTPPVLDS DGSFFLYSKL TVDKSRWQQG NVFSCSVMHE ALHNHYTQKS |
| | | LSLSPGHHHH HH |
| 52 | Fc Hole | EPKSSDKTHT CPPCPAPELL GGPSVFLFPP KPKDTLMISR TPEVTCVVVD |
| | (Empty) Hole | VSHEDPEVKF NWYVDGVEVH NAKTKPREEQ YNSTYRVVSV LTVLHQDWLN |
| | mutations | GKEYKCKVSN KALPAPIEKT ISKAKGQPRE PQVYTLPPCR EEMTKNQVSL |
| | S354C-T366S- | SCAVKGFYPS DIAVEWESNG QPENNYKTTP PVLDSDGSFF LVSKLTVDKS |
| | L368A-Y407V | RWQQGNVFSC SVMHEALHNH YTQKSLSLSP G |
| | bold and | |
| | underlined | |
| 53 | kappa light | GTGGTATCAACGCAGAGTACGCG |
| | chain F primer | |
| 54 | kappa light | GAAGATGAAGACAGATGGTGCAGCCAC |
| | chain R primer | |
| 55 | mouse IgG f | GTGGTATCAACGCAGAGTACGCG |
| | primer | |
| 56 | mouse IgG R | GGGTGCCAGGGGGAAGACSGA |
| | primer | |
| 57 | gp130 VL-CL- | DIQMTQSPSS LSASVGDRVT ITCRASQSVS SSYLAWYQQK PGKAPKLLIY |
| | Fc- (hole) | GTESRATGVP SRFSGSGSGT DFTLTISSLQ PEDFATYYCQ QYGGYPTFGG |
| | | GTKVEIKRTV AAPSVFIFPP SDEQLKSGTA SVVCLLNNFY PREAKVQWKV |
| | | DNALQSGNSQ ESVTEQDSKD STYSLSSTLT LSKADYEKHK VYACEVTHQG |
| | | LSSPVTKSEN RGECDKTHTC PPCPAPEAAGA PSVFLFPPKPK DTLMISRTPEV |
| | | TCVVVDVSHE DPEVKFNWYV DGVEVHNAKT KPREEQYNST YRVVSVLTVL |
| | | HQDWLNGKEY KCKVSNKALP APIEKTISKA KGQPREPQVY TLPPCREEMT |
| | | KNQVSLSCAV KGFYPSDIAV EWESNGQPEN NYKTTPPVLD SDGSFFLVSK |
| | | LTVDKSRWQQ GNVFSCSVMH EALHNHYTQK SLSLSPGK |
| 58 | gp130 VH-CH1 | QVQLVQSGAE VKKPGSSVKV SCKVSGGSIS SYYWSWVRQA PGQGLEWIGR |
| | (mFd) | IYITGNTNYN PSLKDRVTMT VDTSTSTHYM ELSSLRSEDT AVYYCAREGP |
| | | KLGIFDYWGQ GTLVTVSSAS TKGPSVFPLA PSSKSTSGGT AALGCLVKDY |
| | | FPEPVTVSWN SGALTSGVHT FPAVLQSSGL YSLSSVVTVP SSSLGTQTYI |
| | | CNVNHKPSNT KVDKKVEPKS C |
| 59 | IL-27R heavy | EVQLVESGGG LVQPGGSLRL SCAASGFTFE YFGLQWVRQA PGKGLEWIGY |
| | chain (knob) | TSSGGNYEYY EETLKGRFTI SRDNAKNSLY LQMNSLRAED TAVYYCARTK |
| | | TGDYWGQGTL VTVSSASTKG PSVFPLAPSS KSTSGGTAAL GCLVKDYFPE |
| | | PVTVSWNSGA LTSGVHTFPA VLQSSGLYSL SSVVTVPSSS LGTQTYICNV |
| | | NHKPSNTKVD KKVEPKSCDK THTCPPCPAP EAAGAPSVFL FPPKPKDTLM |
| | | ISRTPEVTCV VVDVSHEDPE VKENWYVDGV EVHNAKTKPR EEQYNSTYRV |
| | | VSVLTVLHQD WINGKEYKCK VSNKALPAPI EKTISKAKGQ PREPQVCTLP |
| | | PSREEMTKNQ VSLWCLVKGF YPSDIAVEWE SNGQPENNYK TTPPVLDSDG |
| | | SFFLYSKLTV DKSRWQQGNV FSCSVMHEAL HNHYTQKSLS LSPGK |
| 60 | IL-27R light | RFSGSGSGTD FTLTISSLQP EDFATYYCLQ SKQYPYTFGQ GTKLEIKRTV |
| | chain | AAPSVFIFPP SDEQLKSGTA SVVCLLNNFY PREAKVQWKV DNALQSGNSQ |
| | (identical to | ESVTEQDSKD STYSLSSTLT LSKADYEKHK VYACEVTHQG LSSPVTKSEN RGEC |
| | the IL-27RA | |
| | 4880 LC (SEQ | |
| | ID NO: 14) | |

1. An isolated antibody that specifically binds to IL27RA, comprising a heavy chain variable region (IL27RA-VH) and a light chain variable region (IL27RA-VL), selected from the group consisting of
(i) an antibody comprising a heavy chain variable region (IL27RA-VH) and a light chain variable region (IL27RA-VL), comprising the CDR-H1, CDR-H2, and CDR-H3 sequences of SEQ ID NO: 7, and the CDR-L1, CDR-L2, and CDR-L3 sequences of SEQ ID NO: 8;
(ii) an antibody comprising a heavy chain variable region (IL27RA-VH) and a light chain variable region (IL27RA-VL), comprising a CDR-H1 sequence according to SEQ ID NO: 1; a CDR-H2 sequence according to SEQ ID NO: 2; a CDR-H3 sequence according to SEQ ID NO: 3 and comprising a CDR-L1 sequence according to SEQ ID NO: 4; a CDR-L2 sequence according to SEQ ID NO: 5, and a CDR-L3 sequence according to SEQ ID NO: 6;
(iii) an antibody comprising a IL27RA-VH sequence of SEQ ID NO: 7, comprising a IL27RA-VL sequence of SEQ ID NO: 8;
(iv) an antibody comprising an IL27RA-VH sequence encoded by a nucleic acid sequence of SEQ ID NO: 31 and comprising a, IL27RA-VL sequence encoded by a nucleic acid sequence of SEQ ID NO 32;
(v) an antibody that competes for binding to IL27RA with a second antibody comprising a VH having the amino acid sequence of SEQ ID NO: 7, and a VL having the amino acid sequence of SEQ ID NO: 8; and
(vi) an antibody that specifically binds to IL27RA, comprising a heavy chain variable region (IL27RA-VH) encoded by the plasmid deposited at the ATCC and having ATCC Accession No. PTA-127622, and a light chain variable region (IL27RA-VL) sequence encoded by the plasmid deposited at the ATCC and having ATCC Accession No. PTA-127623.

2. The antibody of claim 1, further comprising an Fc domain, wherein the Fc domain is an isotype of IgA, IgD, IgE, IgM, or IgG, and is optionally a human IgG1 comprising one or more substitutions selected from one or more of the groups consisting of
(i) L234A, L235A, and G237A, by EU numbering, wherein the numbering is according to human IgG1 wildtype;
(ii) D221E & L368E (by EU numbering) wherein the numbering is according to human IgG1 wildtype; and
(iii) D221R & K409R (by EU numbering) wherein the numbering is according to human IgG1 wildtype.

3. The antibody of any claim 1, comprising a heavy chain (HC) and a light chain (LC), selected from the group consisting of
(i) an antibody comprising a heavy chain having the amino acid sequence of SEQ ID NO: 13, and a light chain having the amino acid sequence of SEQ ID NO: 14;
(ii) an antibody comprising a heavy chain having the amino acid sequence of SEQ ID NO: 27, and a light chain having the amino acid sequence of SEQ ID NO: 14; and
(iii) an antibody encoded by an isolated polynucleotide encoding the heavy chain, light chain, or both, of an antibody that binds IL27RA, and wherein said nucleic acid comprises: the nucleic acid sequence of SEQ ID NO: 33 the nucleic acid sequence of SEQ ID NO: 34, or both;
(iv) an antibody encoded by an isolated polynucleotide encoding the heavy chain, light chain, or both, of an antibody that binds IL27RA, and wherein said nucleic acid comprises: the nucleic acid sequence of SEQ ID NO: 39 the nucleic acid sequence of SEQ ID NO: 34, or both; and
(v) an antibody that specifically binds to IL27RA, comprising a heavy chain (IL27RA-HC) encoded by the plasmid deposited at the ATCC and having ATCC Accession No. PTA-127626, and a light chain (IL27RA-LC) sequence encoded by the plasmid deposited at the ATCC and having ATCC Accession No. PTA-127627.

4. The antibody of claim 1, wherein the antibody antagonizes IL27RA.

5. The antibody of claim 1, wherein the antibody further comprises a binding domain to a second target, and the second target is optionally gp130.

6. An isolated antibody that specifically binds to glycoprotein 130 (gp130), comprising a heavy chain variable region (gp130-VH) and a light chain variable region (gp130-VL), comprising one or more selected from the group consisting of:
(i) an antibody comprising the CDR-H1, CDR-H2, and CDR-H3 sequences of SEQ ID NO: 21, and the CDR-L1, CDR-L2, and CDR-L3 sequences of SEQ ID NO: 22;
(ii) an antibody comprising a CDR-H1 sequence according to SEQ ID NO: 15; a CDR-H2 sequence according to SEQ ID NO: 16; a CDR-H3 sequence according to SEQ ID NO: 17 and comprising a CDR-L1 sequence according to SEQ ID NO: 18; a CDR-L2 sequence according to SEQ ID NO: 19, and a CDR-L3 sequence according to SEQ ID NO: 20;
(iii) an antibody comprising a gp130-VH sequence of SEQ ID NO: 21, and comprising a gp130-VL sequence of SEQ ID NO: 22;
(iv) an antibody comprising a gp130-VH sequence encoded by a nucleic acid sequence of SEQ ID NO: 35 and comprising a gp130-VL sequence encoded by a nucleic acid sequence of SEQ ID NO 36;
(v) an antibody that competes for binding to pg130 with a second antibody comprising a VH having the amino acid sequence of SEQ ID NO: 21, and a VL having the amino acid sequence of SEQ ID NO: 22; and
(vi) an antibody comprising a heavy chain variable region (gp130-VH) encoded by the plasmid deposited at the ATCC and having ATCC Accession No. PTA-127624, and a light chain variable region (gp130-VL) sequence encoded by the plasmid deposited at the ATCC and having ATCC Accession No. PTA-127625.

7. The antibody of claim 6, further comprising an Fc domain, wherein the Fc domain is an isotype of IgA, IgD, IgE, IgM, or IgG, and is optionally a human IgG1 comprising one or more substitutions selected from one or more of the groups consisting of
(i) L234A, L235A, and G237A, by EU numbering, wherein the numbering is according to human IgG1 wildtype;
(ii) D221E & L368E (by EU numbering) wherein the numbering is according to human IgG1 wildtype; and
(iii) D221R & K409R (by EU numbering) wherein the numbering is according to human IgG1 wildtype.

8. The antibody of claim 6, comprising a heavy chain (HC) and a light chain (LC), selected from the group consisting of
(i) an antibody comprising a heavy chain having the amino acid sequence of SEQ ID NO: 30 and a light chain having the sequence of SEQ ID NO 24;
(ii) an antibody comprising a heavy chain having the amino acid sequence of SEQ ID NO: 23, and a light chain having the amino acid sequence of SEQ ID NO: 24;
(iii) an antibody encoded by an isolated polynucleotide encoding the heavy chain, light chain, or both, of an antibody that binds gp130, and wherein said nucleic acid comprises: the nucleic acid sequence of SEQ ID NO: 33 the nucleic acid sequence of SEQ ID NO: 34, or both;
(iv) an antibody encoded by an isolated polynucleotide encoding the heavy chain, light chain, or both, of an antibody that binds gp130, and wherein said nucleic acid comprises: the nucleic acid sequence of SEQ ID NO: 40 the nucleic acid sequence of SEQ ID NO: 38, or both; and
(v) an antibody that specifically binds to gp130, comprising a heavy chain (gp130-HC) encoded by the plasmid deposited at the ATCC and having ATCC Accession No. PTA-127628, and a light chain (gp130-LC) sequence encoded by the plasmid deposited at the ATCC and having ATCC Accession No. PTA-127629.

9. The antibody of claim 6, wherein the antibody further comprises a binding domain to a second target, and the second target is optionally IL27RA.

10. An isolated antibody comprising a first antigen binding site that binds to IL27RA and a second antigen binding site that binds to gp130, wherein the first antigen binding site comprises the antibody according to claim 1.

11. An isolated antibody comprising a first antigen binding site that binds to IL27RA and a second antigen binding site that binds to gp130 as claimed in claim 6, wherein the first antigen binding site comprises a IL27RA-VH and an (L27RA-VL, wherein the second antigen binding site comprises a gp130-VH and a gp130-VL, selected from the group consisting of:
(i) an antibody wherein
a) the IL27RA-VH comprises (i) a VH CDR1 comprising the amino acid sequence of SEQ ID NO: 1 (ii) a VH CDR2 comprising the amino acid sequence of SEQ ID NO: 2; and (iii) a VH CDR3 comprising the amino acid sequence of SEQ ID NO: 3;
b) the IL27RA-VL comprises (i) a VL CDR1 comprising the amino acid sequence of SEQ ID NO: 4; (ii) a VL CDR2 comprising the amino acid sequence of SEQ ID NO: 5; and (iii) a VL CDR3 comprising the amino acid sequence of SEQ ID NO: 6;
c) the gp130-VH comprises (i) a VH CDR1 comprising the amino acid sequence of SEQ ID NO: 15; (ii) a VH CDR2 comprising the amino acid sequence of SEQ ID NO: 16; and (iii) a VH CDR3 comprising the amino acid sequence of SEQ ID NO: 17;
d) and the gp130-VL comprises (i) a VL CDR1 comprising the amino acid sequence of SEQ ID NO: 18; (ii) a VL CDR2 comprising the amino acid sequence of SEQ ID NO: 19; and (iii) a VL CDR3 comprising the amino acid sequence of SEQ ID NO: 20;
(ii) an antibody comprising a IL27RA-VH comprising the amino acid sequence of SEQ ID NO: 7, a IL27RA-VL comprising the amino acid sequence of SEQ ID NO: 8, a gp130-VL comprising the amino acid sequence of SEQ ID NO: 21, and gp130-VL comprising the amino acid sequence of SEQ ID NO: 22; and
(iii) an antibody comprising a IL27RA-VH encoded by the plasmid deposited at the ATCC and having ATCC Accession No. PTA-127622, and a IL27RA-VL sequence encoded by the plasmid deposited at the ATCC and having ATCC Accession No. PTA-127623, and further comprising a gp130-VH encoded by the plasmid deposited at the ATCC and having ATCC Accession No. PTA-127624, and a light chain variable region (gp130-VL) sequence encoded by the plasmid deposited at the ATCC and having ATCC Accession No. PTA-127625.

12. The antibody of claim 11, further comprising an Fc domain, wherein the Fc domain is an isotype of IgA, IgD, IgE, IgM, or IgG, and is optionally a human IgG1 comprising one or more substitutions selected from one or more of the groups consisting of
(i) L234A, L235A, and G237A, by EU numbering, wherein the numbering is according to human IgG1 wildtype;
(ii) D221E & L368E (by EU numbering) wherein the numbering is according to human IgG1 wildtype; and
(iii) D221R & K409R (by EU numbering) wherein the numbering is according to human IgG1 wildtype.

13. The antibody of claim 12, wherein the antibody comprises a Fc domain comprising a first and second Fc chain, that each contain one or more amino acid modifications that promote the association of the first Fc chain with the second Fc chain.

14. The antibody of claim 13, wherein the first Fc chain comprises an amino acid modification at positions 221 and 409 (EU numbering) of human IgG1, and wherein the second Fc chain comprises an amino acid modification at positions 221 and 368 (by EU numbering) of human IgG1, and where optionally, the modifications are D221R & K409R in the first chain, and D221E & L368E in the second chain.

15. An isolated antibody comprising a first antigen binding site that binds to IL27RA and a second antigen binding site that binds to gp130, wherein the first antigen binding site comprises a VH and a VL, wherein the second antigen binding site comprises a VH and a VL, and wherein the antibody has a binding affinity for human IL27RA at least two orders of magnitude lower the antibody binding affinity to human gp130.

16. The antibody as claimed in claim 10, selected from the group consisting of:
(i) an antibody comprising a first heavy chain and a first light chain, and a second heavy chain and second light chain, wherein the first heavy chain and the first light chain comprise a first antigen binding site that binds to IL27RA, and the second heavy chain and the second light chain comprise a second antigen binding site that binds to gp130, wherein the first antibody heavy chain comprises the amino acid sequence of SEQ ID NO:27, the first antibody light chain comprises the amino acid sequence of SEQ ID NO: 14, the second antibody heavy chain comprises the amino acid sequence of SEQ ID NO: 30, and the second antibody light chain comprises the amino acid sequence of SEQ ID NO: 34; and
(ii) an antibody comprising a heavy chain (IL27RA-HC) encoded by the plasmid deposited at the ATCC and having ATCC Accession No. PTA-127626, and a light chain (IL27RA-LC) sequence encoded by the plasmid deposited at the ATCC and having ATCC Accession No. PTA-127627, and further comprising a heavy chain (gp130-VH) encoded by the plasmid deposited at the ATCC and having ATCC Accession No. PTA-127628, and a light chain (gp130-LC) sequence encoded by the plasmid deposited at the ATCC and having ATCC Accession No. PTA-127629.

17. The antibody of claim 16, wherein the antibody has one or more characteristics selected from the group consisting of:
(i) a higher binding affinity for IL27RA than for gp130 as measured by SPR;
(ii) a binding affinity at least 10-fold higher for IL27RA than for gp130 as measured by SPR;
(iii) the antibody binds to human IL27RA with an affinity of less than 1 nM when measured by SPR;
(iv) the antibody binds to human gp130 with an affinity of less than 1000 nM when measured by SPR;
(v) the antibody agonizes IL27RA;
(vi) the antibody binds cynomolgus IL27RA;
(vii) the binding KD of the antibody cynomolgus IL27RA is within 10 orders of magnitude of the binding KD of the antibody to human IL27RA as measured by SPR;
(viii) the antibody antagonizes gp130;
(ix) the antibody binds cynomolgus gp130;
(x) the binding KD of the antibody to cynomolgus gp130 is within 3 orders of magnitude of the binding KD of the antibody to human gp130 as measured by SPR;
(xi) the antibody has a binding affinity at least 100-fold higher for IL27RA than for gp130 as measured by SPR;
(xii) the antibody binds to human IL27RA with an affinity of less than 1 nM when measured by SPR;
(xiii) the antibody binds to human gp130 with an affinity of less than 1000 nM when measured by SPR;
(xiv) the antibody binds to human IL27RA with an affinity of between 0.01 nm and 5 nM and binds to human gp130 with an affinity of between 10 nm and 1000 nM, when measured by SPR;
(xv) the antibody is characterized by an EC50 of less than 10 nm in a phosphorylated STAT1 CD3+ T cell fluorescent flow cytometry assay
(xvi) the antibody is characterized by an EC50 of less than 5 nm in a flow cytometry assay of phosphorylated STAT1 CD3+ T cells;
(xvii) the antibody is capable of down-regulating pathogenic cytokine production
(xviii) the antibody is capable of down-regulating 11-17 production in T helper cells;
(xix) the antibody is characterized by an IC50 of less than 0.05 nm as measured by Il-17 immunoassay;
(xx) the antibody is capable of promoting regulatory T cell differentiation;
(xxi) the antibody is capable of upregulating indoleamine-pyrrole 2,3-dioxygenase (I Dal) expression;
(xxii) the antibody is capable of upregulating indoleamine-pyrrole 2,3-dioxygenase (IDO1) expression in CD14+ human monocytes and/or human colonocytes; and
(xxiii) the antibody is characterized by an EC50 of less than 100 nm by LC-MS assay determination of kynurenine production.

18. A pharmaceutical composition comprising a therapeutically effective amount of the antibody of claim 16 and a pharmaceutically acceptable carrier.

19. The antibody of claim 16, for use in the treatment of a disease, and where optionally, the inflammatory disease is one or more selected from the group consisting of an inflammatory disease, inflammatory bowel disease (IBD), Crohn's disease (CD), ulcerative colitis (UC), multiple sclerosis, rheumatoid arthritis, celiac disease, asthma, allergic diseases, obesity, type 2 diabetes, and cancer.

20. A method of treating a medical condition, comprising administering to a subject in need thereof a therapeutically effective amount of the antibody of claim 16, and where optionally, the medical condition is one or more selected from the group consisting of an inflammatory disease, inflammatory bowel disease (IBD), Crohn's disease (CD), ulcerative colitis (UC), multiple sclerosis, rheumatoid arthritis, celiac disease, asthma, allergic diseases, obesity, type 2 diabetes, and cancer.

21. An isolated polynucleotide encoding the antibody of claim 16.

22. An isolated polynucleotide comprising one or more selected from the group consisting of
(i) a polynucleotide encoding the VH of an antibody that binds IL27RA, wherein the polynucleotide comprises the nucleic acid sequence of SEQ ID NO 31;
(ii) a polynucleotide encoding the VL of an antibody that binds IL27RA, wherein the polynucleotide comprises the nucleic acid sequence of SEQ ID NO: 32;
(iii) a polynucleotide encoding the VH and VL of an antibody that binds IL27RA wherein the polynucleotide encoding the VH comprises the nucleic acid sequence of SEQ ID NO: 31 and the polynucleotide encoding the VL comprises the nucleic acid sequence of SEQ ID NO: 32;
(iv) a polynucleotide encoding the heavy chain, light chain, or both, of an antibody that binds IL27RA, and wherein said nucleic acid comprises: the nucleic acid sequence of SEQ ID NO: 33 the nucleic acid sequence of SEQ ID NO: 34, or both.
(v) a isolated polynucleotide encoding the heavy chain, light chain, or both, of an antibody that binds IL27RA, and wherein said nucleic acid comprises: the nucleic acid sequence of SEQ ID NO: 39 the nucleic acid sequence of SEQ ID NO: 34, or both;
(vi) a polynucleotide encoding the VH of an antibody that binds gp130, wherein the polynucleotide comprises the nucleic acid sequence of SEQ ID NO: 35;
(vii) a polynucleotide encoding the VL of an antibody that binds gp130, wherein the polynucleotide comprises the nucleic acid sequence of SEQ ID NO: 36;
(viii) a polynucleotide encoding the VH and VL of an antibody that binds gp130, wherein the polynucleotide encoding the VH comprises the nucleic acid sequence of SEQ ID NO: 35 and the polynucleotide encoding the VL comprises the nucleic acid sequence of SEQ ID NO: 36;
(ix) a polynucleotide encoding the heavy chain, light chain, or both, of an antibody that binds gp130, and wherein said polynucleotide comprises: the nucleic acid sequence of SEQ ID NO: 37, the nucleic acid sequence of SEQ ID NO: 38, or both;
(x) a polynucleotide encoding the heavy chain, light chain, or both, of an antibody that binds gp130, and wherein said nucleic acid comprises: the nucleic acid sequence of SEQ ID NO: 40, the nucleic acid sequence of SEQ ID NO: 38, or both;
(xi) a polynucleotide encoding a IL27RA-VH encoded by the plasmid deposited at the ATCC and having ATCC Accession No. PTA-127622.
(xii) IL27RA-VL encoded by the plasmid deposited at the ATCC and having ATCC Accession No. PTA-127623.
(xiii) a polynucleotide encoding a IL27RA-VH encoded by the plasmid deposited at the ATCC and having ATCC Accession No. PTA-127622, and a IL27RA-VL sequence encoded by the plasmid deposited at the ATCC and having ATCC Accession No. PTA-127623;
(xiv) a polynucleotide encoding a IL27RA-HC encoded by the plasmid deposited at the ATCC and having ATCC Accession No. PTA-127626;
(xv) a polynucleotide encoding a IL27RA-LC encoded by the plasmid deposited at the ATCC and having ATCC Accession No. PTA-127627;
(xvi) a polynucleotide encoding a IL27RA-HC encoded by the plasmid deposited at the ATCC and having ATCC Accession No. PTA-127626, and a IL27RA-LC sequence encoded by the plasmid deposited at the ATCC and having ATCC Accession No. PTA-127627;
(xvii) a polynucleotide encoding a gp130-VH encoded by the plasmid deposited at the ATCC and having ATCC Accession No. PTA-127624;
(xviii) a polynucleotide encoding a gp130-VL encoded by the plasmid deposited at the ATCC and having ATCC Accession No. PTA-127625;
(xix) a polynucleotide encoding a gp130-VH encoded by the plasmid deposited at the ATCC and having ATCC Accession No. PTA-127624, and a gp130-VL sequence encoded by the plasmid deposited at the ATCC and having ATCC Accession No. PTA-127625;
(xx) a polynucleotide encoding a gp130-HC encoded by the plasmid deposited at the ATCC and having ATCC Accession No. PTA-127628;
(xxi) a polynucleotide encoding a gp130-LC encoded by the plasmid deposited at the ATCC and having ATCC Accession No. PTA-127629; and
(xxii) a polynucleotide encoding a gp130-VH) encoded by the plasmid deposited at the ATCC and having ATCC Accession No. PTA-127628, and a gp130-LC sequence encoded by the plasmid deposited at the ATCC and having ATCC Accession No. PTA-127629.

23. A vector comprising the polynucleotide of any one of claim 22.

24. An isolated host cell comprising the polynucleotide of claim 22, or the vector of claim 23.

25. A method of producing an isolated antibody, comprising culturing the host cell of claim 24 under conditions that result in production of the antibody, and recovering the antibody.
